SEQUENCE LISTING (37 sequences)

SEQ ID NO: 1 - protein, 25 aa, synthetic construct
  REGION 1..25: note = signal peptide (TPA)
MDAMLRGLCCVLLLCGAVFVSPSHA

SEQ ID NO: 2 - protein, 208 aa, synthetic construct [sequence omitted]

SEQ ID NO: 3 - protein, 30 aa, synthetic construct
  REGION 1..30: note = hinge
GSGGGGSGGGGSGGGGSAESKYGPPCPPCP

SEQ ID NO: 4 - protein, 216 aa, synthetic construct
  REGION 1..216: note = immunoglobulin fc
APEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 5 - protein, 5 aa, synthetic construct
  REGION 1..5: note = linker
GGGGS

SEQ ID NO: 6 - protein, 133 aa, synthetic construct
  REGION 1..133: note = hIL-2M
APTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 7 - protein, 617 aa, synthetic construct
  REGION 1..617: note = fusion protein comprising variants of IL-2 and fragments of CD80
MDAMLRGLCCVLLLCGAVFVSPSHAVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - DNA, 1857 bp, synthetic construct
  misc_feature 1..1857: note = nucleotiedes coding fusion protein (GI101)
  source 1..1857: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacctgatga

SEQ ID NO: 9 - protein, 592 aa, synthetic construct
  REGION 1..592: note = fusion protein (GI101)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, synthetic construct [sequence omitted]

SEQ ID NO: 11 - protein, 288 aa, synthetic construct
  REGION 1..288: note = CD80
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 12 - protein, 215 aa, synthetic construct
  REGION 1..215: note = modified Fc
SHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 306 aa, synthetic construct
  REGION 1..306: note = mCD80
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 14 - DNA, 1848 bp, synthetic construct
  misc_feature 1..1848: note = nucleotiedes coding fusion protein (mGI101)
  source 1..1848: mol_type = other DNA
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtctcttggaggtggtggcggttctgcccctacctccagctctaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacttgcagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagagcatcatctccacactgacc

SEQ ID NO: 15 - protein, 616 aa, synthetic construct
  REGION 1..616: note = fusion protein (mGI101)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - DNA, 1437 bp, synthetic construct
  misc_feature 1..1437: note = nucleotiedes coding fusion protein (GI101C1)
  source 1..1437: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtccctgggc

SEQ ID NO: 17 - protein, 454 aa, synthetic construct
  REGION 1..454: note = fusion protein (GI101C1)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 18 - DNA, 1176 bp, synthetic construct
  misc_feature 1..1176: note = nucleotiedes coding fusion protein (GI101C2)
  source 1..1176: mol_type = other DNA
atggatgctatgctgagaggcctgtgttgcgtgctgctgctgtgtggcgctgtgttcgtgtctccatctcacgccgctgagtctaagtacggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggccctgcacaatcactacacccagaagtccctgtctctgtctcttggcggaggcggaggatctgctcctacctccagctccaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacctccagtgcctggaagaggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagtccatcatctccacactgacc

SEQ ID NO: 19 - protein, 367 aa, synthetic construct
  REGION 1..367: note = fusion protein (GI101C2)
AESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - DNA, 1434 bp, synthetic construct
  misc_feature 1..1434: note = nucleotiedes coding fusion protein (mGI101C1)
  source 1..1434: mol_type = other DNA
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtccctgggc

SEQ ID NO: 21 - protein, 478 aa, synthetic construct
  REGION 1..478: note = fusion protein (mGI101C1)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLG

SEQ ID NO: 22 - protein, 133 aa, synthetic construct
  REGION 1..133: note = variants of IL-2 (3M, M45)
APTSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 23 - protein, 133 aa, synthetic construct
  REGION 1..133: note = variants of IL-2 (3M, M61)
APTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 24 - protein, 133 aa, synthetic construct
  REGION 1..133: note = variants of IL-2 (3M, M72)
APTSSSTKKTQLQLEHLLLDLQMILNGINNYKNPKLTAMLTAKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 25 - DNA, 1851 bp, synthetic construct
  misc_feature 1..1851: note = nucleotiedes coding fusion protein (GI102-M45)
  source 1..1851: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttcgccatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 26 - protein, 592 aa, synthetic construct
  REGION 1..592: note = fusion protein (GI102-M45)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFAMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - DNA, 1851 bp, synthetic construct
  misc_feature 1..1851: note = nucleotiedes coding fusion protein (GI102-M61)
  source 1..1851: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaaagggaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 28 - protein, 592 aa, synthetic construct
  REGION 1..592: note = fusion protein (GI102-M61)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 29 - DNA, 1857 bp, synthetic construct
  misc_feature 1..1857: note = nucleotiedes coding fusion protein (GI102-M72)
  source 1..1857: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgaccgccatgctgaccgctaagttctacatgcccaagaaggccaccgagctgaagcacctccagtgcctggaagaagaactgaagcccctggaagaggtgctgaatggggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacctgatga

SEQ ID NO: 30 - protein, 592 aa, synthetic construct
  REGION 1..592: note = fusion protein (GI102-M72)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLEEELKPLEEVLNGAQSKNFHLRPRDLISNINVIVLELKGSETTFMCEYADETATIVEFLNRWITFCQSIISTLT

SEQ ID NO: 31 - DNA, 1851 bp, synthetic construct
  misc_feature 1..1851: note = nucleotiedes coding fusion protein (GI101w)
  source 1..1851: mol_type = other DNA
atggatgctatgctgagaggcctgtgttgcgtgctgctgctgtgtggcgctgtgttcgtgtctccttctcacgctgtgatccacgtgaccaaagaagtgaaagaggtcgccacactgtcctgcggccacaacgtttcagtggaagaactggcccagaccaggatctactggcagaaagaaaagaaaatggtgctgaccatgatgtccggcgacatgaacatctggcctgagtacaagaaccggaccatcttcgacatcaccaacaacctgtccatcgtgattctggccctgaggccttctgatgagggcacctatgagtgcgtggtgctgaagtacgagaaggacgccttcaagcgcgagcacctggctgaagtgacactgtccgtgaaggccgactttcccacaccttccatctccgacttcgagatccctacctccaacatccggcggatcatctgttctacctctggcggctttcctgagcctcacctgtcttggctggaaaacggcgaggaactgaacgccatcaacaccaccgtgtctcaggaccccgaaaccgagctgtacgctgtgtcctccaagctggacttcaacatgaccaccaaccacagcttcatgtgcctgattaagtacggccacctgagagtgaaccagaccttcaactggaacaccaccaagcaagagcacttccctgacaatggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacacccgaagtgacctgcgtggtggtggatgtgtctcaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctagatggcaagagggcaacgtgttctcctgctctgtgctgcacgaggccctgcacaatcactacacccagaagtccctgtctctgtctcttggaggtggtggcggttctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

SEQ ID NO: 32 - protein, 592 aa, synthetic construct
  REGION 1..592: note = fusion protein (GI101w)
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 33 - DNA, 1848 bp, synthetic construct
  misc_feature 1..1848: note = nucleotiedes coding fusion protein (mGI102-M61)
  source 1..1848: mol_type = other DNA
atggatgctatgctgagaggcctgtgttgcgtgctgctgctgtgtggcgctgtgttcgtgtctccttctcacgctgtggacgagcagctctccaagtccgtgaaggataaggtcctgctgccttgccggtacaactctcctcacgaggacgagtctgaggaccggatctactggcagaaacacgacaaggtggtgctgtccgtgatcgccggaaagctgaaagtgtggcctgagtacaagaacaggaccctgtacgacaacaccacctacagcctgatcatcctgggcctcgtgctgagcgatagaggcacctattcttgcgtggtgcagaagaaagagcggggcacctacgaagtgaagcacctggctctggtcaagctgtccatcaaggccgacttcagcacccctaacatcaccgagtctggcaacccttccgccgacaccaagagaatcacctgtttcgcctctggcggcttccctaagcctcggttctcttggctggaaaacggcagagagctgcccggcatcaataccaccatttctcaggacccagagtccgagctgtacaccatctccagccagctcgactttaacaccaccagaaaccacaccatcaagtgcctgattaagtacggcgacgcccacgtgtccgaggactttacttgggagaaacctcctgaggaccctcctgactctggatctggcggcggaggttctggcggaggtggaagcggaggcggaggatctgctgagtctaagtatggccctccttgtcctccatgtcctgctccagaagctgctggcggaccctctgtgttcctgtttcctccaaagcctaaggaccagctcatgatctctcggacccctgaagtgacctgcgtggtggtggatgtgtctcaagaggaccctgaggtgcagttcaattggtacgtggacggcgtggaagtgcacaacgccaagaccaagcctagagaggaacagttcaactccacctatagagtggtgtccgtgctgaccgtgctgcaccaggattggctgaacggcaaagagtacaagtgcaaggtgtccaacaagggcctgccttccagcatcgaaaagaccatcagcaaggctaagggccagcctagggaaccccaggtttacaccctgcctccaagccaagaggaaatgaccaagaaccaggtgtccctgacctgcctggtcaagggcttctacccttccgacattgccgtggaatgggagtccaatggccagcctgagaacaactacaagaccacacctcctgtgctggactccgacggctccttctttctgtactctcgcctgaccgtggacaagtctaggtggcaagagggcaacgtgttctcctgctctgtgctgcacgaggctctgcacaaccactacacccagaagtccctgtctctgtctcttggaggtggtggcggttctgcccctacctccagctctaccaagaaaacccagctccagttggagcatctgctgctggacctccagatgatcctgaatggcatcaacaattacaagaaccccaagctgaccgccatgctgaccgctaagttctacatgcccaagaaggccaccgagctgaagcacttgcagtgcctggaaagggaactgaagcccctggaagaagtgctgaatctggcccagtccaagaacttccacctgaggcctagggacctgatctccaacatcaacgtgatcgtgctggaactgaaaggctccgagacaaccttcatgtgcgagtacgccgacgagacagccaccatcgtggaatttctgaaccggtggatcaccttctgccagagcatcatctccacactgacc

SEQ ID NO: 34 - protein, 616 aa, synthetic construct
  REGION 1..616: note = fusion protein (mGI102-M61)
MDAMLRGLCCVLLLCGAVFVSPSHAVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSGSGGGGSGGGGSGGGGSAESKYGPPCPPCPAPEAAGGPSVFLFPPKPKDQLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGGGGGSAPTSSSTKKTQLQLEHLLLDLQMILNGINNYKNPKLTAMLTAKFYMPKKATELKHLQCLERELKPLEEVLNLAQSKNFHLRPRDLISNINVIVLELKGSETTFMCEYADETATIVEFLNRWITFCQSIISTLT

SEQ ID NO: 35 - protein, 153 aa, synthetic construct
  REGION 1..153: note = wild type hIL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 36 - protein, 158 aa, synthetic construct
  REGION 1..158: note = IL-2 with signal sequence
MDAMLRGLCCVLLLCGAVFVSPSHAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 37 - DNA, 474 bp, synthetic construct
  misc_feature 1..474: note = nucleotide sequence coding IL-2 with signal sequence
  source 1..474: mol_type = other DNA
atggatgctatgctgagaggcctgtgttgcgtgctgctgctgtgtggcgctgtgttcgtgtctccttctcacgctgcccctaccagctcctctaccaagaaaacccagctccagttggagcatctgctgctggacctccagatgattctgaacgggatcaacaactataagaaccccaagctgacccgcatgctgacctttaagttctacatgcccaagaaggccaccgagctgaagcacctccagtgcctggaagaagaactgaagcccctggaagaggtgctgaatctggcccagtccaagaacttccacctgaggccacgggacctgatcagcaacatcaacgtgatcgtgctggaactgaagggctccgagacaacctttatgtgcgagtacgccgacgagacagccaccatcgtggaatttctgaaccggtggatcaccttctgccagagcatcatctccacactgacc

(19) United States
(12) Patent Application Publication
(10) Pub. No.: US 2023/0104217 A1
(43) Pub. Date: Apr. 6, 2023

(54) FUSION PROTEIN COMPRISING IL-2 PROTEIN AND CD80 PROTEIN, AND USE THEREOF

(71) Applicant: GI INNOVATION, INC., Seoul (KR)

(72) Inventor: Myung Ho JANG, Seoul (KR)

(73) Assignee: GI INNOVATION, INC., Seoul (KR)

(21) Appl. No.: 17/948,894

(22) Filed: Sep. 20, 2022

Related U.S. Application Data
(63) Continuation-in-part of application No. 16/959,312, filed on Jun. 30, 2020, now Pat. No. 11,492,384.
(60) Provisional application No. 62/832,013, filed on Apr. 10, 2019.

(30) Foreign Application Priority Data
Sep. 17, 2018 (KR) 10-2018-0110698
Jan. 7, 2019 (KR) 10-2019-0001867
May 8, 2019 (KR) 10-2019-0053436

(51) Int. Cl. C07K 14/55; C07K 14/705

(52) U.S. Cl. CPC C07K 14/55; C07K 14/70532; C12N 5/0638

(57) ABSTRACT

Provided is a fusion protein comprising IL-2 protein and CD80 protein, a fusion protein having a high content of sialic acid, and a pharmaceutical composition containing same. A fusion protein containing CD80 fragment, immunoglobulin Fc, and an IL-2 variant can activate immune cells, such as natural killer cells, and at the same time, can control immune cell regulatory activity of regulatory T cells. In addition, the fusion protein having a high content of sialic acid can proliferate immune cells, such as lymphocytes including CD8+ T cells and natural killer cells. Therefore, a pharmaceutical composition containing the fusion protein as an active ingredient is very industrially useful in that such pharmaceutical composition can increase immune activity in the body, and thus can be effectively used against infectious diseases as well as cancer.

### CROSS REFERENCE TO RELATED APPLICATIONS

This application is a Continuation-in-Part of U.S. application Ser. No. 16/959,312 filed Jun. 30, 2020 (allowed), which is a National Stage of International Application No. PCT/KR2019/011928 filed Sep. 16, 2019, claiming priority based on Korean Patent Application No. 10-2018-0110698 filed Sep. 17, 2018, Korean Patent Application No. 10-2019-0001867 filed Jan. 7, 2019, U.S. Provisional Patent Application No. 62/832,013 filed Apr. 10, 2019, and Korean Patent Application No. 10-2019-0053436 filed May 8, 2019.

### SEQUENCE LISTING

The content of the electronically submitted sequence listing, file name: Sequence_Listing_As_Filed.xml; size: 73,154 bytes; and date of creation: Sep. 20, 2022, filed herewith, is incorporated herein by reference in its entirety.

### TECHNICAL FIELD

The present invention relates to a fusion protein comprising an IL-2 protein and a CD80 protein, and a use thereof. Specifically, the present invention relates to a novel fusion protein having cancer therapeutic and immunopotentiating efficacy and having a high content of sialic acid.

### BACKGROUND ART

Interleukin 2 (IL-2), also called T-cell growth factor (TCGF), is a globular glycoprotein that plays a central role in lymphocyte production, survival, and homeostasis. IL-2 has a protein size of 15.5 kDa to 16 kDa and consists of 133 amino acids. IL-2 mediates various immune actions by binding to an IL-2 receptor composed of three distinct subunits.

In addition, IL-2 is synthesized mainly by activated T cells, in particular by CD4+ helper T cells. IL-2 stimulates proliferation and differentiation of T cells, and induces production of cytotoxic T lymphocytes (CTLs) and differentiation of peripheral blood lymphocytes into cytotoxic cells and lymphokine-activated killer cells (LAK cells).

Furthermore, IL-2 is involved in proliferation and differentiation of B cells, promotes immunoglobulin synthesis by B cells, and stimulates production, proliferation, and activation of natural killer cells (NK cells). Therefore, IL-2 is used as an anticancer agent, because it can increase lymphocyte populations and increase the function of the immune cells in the living body. Currently, therapy with IL-2 has been approved and used for patients with metastatic renal cell carcinoma and malignant melanoma.

However, IL-2 has a dual function in immune responses in that it is important not only for mediating an increase in number of immune cells and activity thereof, but also for maintaining immune tolerance. In addition, it has been reported that IL-2 may not be optimal for inhibiting tumor growth. The reason is that in the presence of IL-2, activation-induced cell death (AICD) may occur in the resulting cytotoxic T lymphocytes and immune responses may be inhibited by IL-2-dependent regulatory T cells (Treg cells) (Imai et al., Cancer Sci 98, 416-423, 2007).

In addition, severe cardiovascular, pulmonary, renal, hepatic, gastrointestinal, neuronal, cutaneous, hematological, and systemic side effects occur in patients who have received immunotherapy with IL-2. Therefore, various IL-2 mutations have been studied to improve therapeutic efficacy of IL-2 and minimize side effects thereof (U.S. Pat. No. 5,229,109 B). However, there are still many problems to be solved in order to utilize IL-2 for pharmacological purposes.

Meanwhile, CD80, also known as B7-1, is a member of the B7 family of membrane-bound proteins that are involved in immune regulation by binding to its ligand by way of delivering costimulatory responses and coinhibitory responses. CD80 is a transmembrane protein expressed on the surface of T cells, B cells, dendritic cells, and monocytes. CD80 is known to bind CD28, CTLA4 (CD152), and PD-L1. CD80, CD86, CTLA4, and CD28 are involved in a costimulatory-coinhibitory system. For example, they regulate activity of T cells and are involved in proliferation, differentiation, and survival thereof.

For example, when CD80 and CD86 interact with CD28, costimulatory signals are generated to activate T cells. Eventually, CD80 binds to CTLA4 and stimulates CTLA4 to be upregulated. As a result, CD80 inhibits T cell responses prior to immune response activation caused by CD80/CD28 interaction. This feedback loop allows for fine regulation of immune responses.

In addition, CD80 is known to bind PD-L1, another B7 family member, with affinity similar to that with which CD28 binds PD-L1. PD-L1 is known as one of two ligands for programmed death-1 (PD-1) protein, and PD-L1 is known to be involved in T cell regulation. Binding of CD80 to PD-L1 is another mechanism that can block PD-1/PD-L1 interaction, which may prevent inhibition of T cell responses in tumors. At the same time, however, an increase in CD80 levels causes CD80 to bind to CD28 so that CTLA4 is induced, thereby inducing or inhibiting T cell responses.

### DISCLOSURE

### Technical Problem

The present inventors have studied to develop IL-2 which is safe and effective. As a result, the present inventors have discovered that a novel fusion protein comprising, in one molecule, an IL-2 protein and a CD80 protein can activate immune cells and effectively regulate Treg cells, and the fusion protein having a high content of sialic acid can proliferate immune cells, such as lymphocytes including CD8+ T cells and natural killer cells, thereby completing the present invention.

### Solution to Problem

In order to achieve the above object, in an aspect of the present invention, there is provided a fusion protein dimer comprising two monomers, each of which contains the following structural formula (I) or (II), wherein the fusion protein dimer comprising sialic acid and a molar ratio of sialic acid to the fusion protein dimer is at least 7.

In another aspect of the present invention, there is provided a pharmaceutical composition comprising the fusion protein dimer.

In yet another aspect of the present invention, there is provided a method for enhancing immunity in a subject by using the fusion protein dimer or the pharmaceutical composition.

### Advantageous Effects

A fusion protein comprising an IL-2 protein and a CD80 protein can not only activate immune cells owing to IL-2, but also effectively regulate Treg cells owing to CD80. In addition, the fusion protein having a high content of sialic acid can proliferate immune cells, such as lymphocytes including CD8+ T cells and natural killer cells. Therefore, the fusion protein can attack cancer cells in an efficient manner, and thus can be usefully employed for treatment of cancer or an infectious disease.

### BRIEF DESCRIPTION OF THE DRAWINGS

FIG. 1 illustrates a schematic embodiment of a fusion protein.

FIG. 2 illustrates a mechanism by which the fusion protein regulates two different types of immune cells; however, it should be understood that the mechanism by which the action of the fusion protein is expressed is not limited thereto.

FIG. 3 illustrates a mechanism by which the fusion protein exhibits an anticancer effect.

FIG. 4 illustrates a schematic view of the structure of the fusion protein. Here, each of GI101 and mGI101 is an embodiment of the fusion protein herein, and GI101C1, GI101C2, and mGI101C1 are comparative examples for comparison with activity of the fusion protein.

FIG. 5 illustrates various embodiments of the fusion protein herein. Human- and mouse-derived proteins may be combined to prepare a fusion protein. CD80 protein and IL-2 protein may be bound to each other via various linkers other than Fc.

FIG. 6 illustrates a result obtained by identifying the obtained fusion protein (GI101) with SDS-PAGE.

FIG. 7 illustrates amounts of the fusion protein (GI101) depending on absorbance.

FIG. 8 illustrates a result obtained by analyzing the obtained fusion protein (GI101) by size exclusion chromatography (SEC).

FIG. 9 illustrates a result obtained by identifying the obtained mGI101 fusion protein with SDS-PAGE.

FIG. 10 illustrates results obtained by identifying the obtained GI101C1 fusion protein with SDS-PAGE.

FIG. 11 illustrates results obtained by identifying the obtained GI101C2 fusion protein with SDS-PAGE.

FIG. 12 illustrates a result obtained by identifying the obtained mGI101C1 fusion protein with SDS-PAGE.

FIG. 13 illustrates results obtained by identifying the obtained GI102-M45 fusion protein with SDS-PAGE.

FIG. 14 illustrates results obtained by identifying the obtained GI102-M61 fusion protein with SDS-PAGE.

FIG. 15 illustrates results obtained by identifying the obtained GI102-M72 fusion protein with SDS-PAGE.

FIG. 16 illustrates binding affinity between hCTLA4 and GI101.

FIG. 17 illustrates binding affinity between hPD-L1 and GI101.

FIG. 18 illustrates binding affinity between hPD-L1 and hPD-1.

FIG. 19 illustrates binding affinity between mCTLA4 and mGI101.

FIG. 20 illustrates binding affinity between mPD-L1 and mGI101.

FIGS. 21 and 22 illustrate results obtained by identifying binding ability between GI-101 (hCD80-Fc-h1L-2v) and CTLA-4, and between GI-101 (hCD80-Fc-hIL-2v) and PD-L1. It was identified that GI-101 (hCD80-Fc-hIL-2v) has high binding ability for CTLA-4 and PD-L1.

FIG. 23 illustrates an effect of GI101 on PD-1/PD-L1 binding. GI101 effectively inhibited PD-1/PD-L1 binding.

FIG. 24 illustrates results obtained by identifying binding affinity between GI101 and IL-2Rα or IL-2Rβ.

FIG. 25 illustrates results obtained by identifying binding affinity between GI101 and IL-2Rα.

FIG. 26 illustrates results obtained by identifying binding affinity between GI101 and IL-2Rβ.

FIG. 27 illustrates results obtained by identifying binding affinity between IL-2Rα and GI102-M45.

FIG. 28 illustrates results obtained by identifying binding affinity between IL-2Rα and GI102-M61.

FIG. 29 illustrates results obtained by identifying binding affinity between IL-2Rα and GI102-M72.

FIG. 30 illustrates results obtained by identifying binding affinity between IL-2Rβ and GI102-M45.

FIG. 31 illustrates results obtained by identifying binding affinity between IL-2Rβ and GI102-M61.

FIG. 32 illustrates results obtained by identifying binding affinity between IL-2Rβ and GI102-M72.

FIGS. 33 and 34 illustrate results obtained by measuring amounts of IFN-γ secreted from cells when the cells are subjected to treatment with GI101, GI101C1, GI101C2, or IL-2 at respective concentrations and incubation is performed.

FIGS. 35 and 36 illustrate results obtained by identifying effects of GI101, GI101C1, GI101C2, and IL-2 (Proleukin) on proliferation of CD8+ T cells.

FIGS. 37A to 37C illustrate results obtained by identifying effects of GI101 and GI102 on proliferation of CD8+ T cells and CD4+ T cells. Here, FIG. 37A illustrates proportions of CD8+ T cells and CD4+ T cells, FIG. 37B illustrates proliferation capacity of CD8+ T cells, and FIG. 37C illustrates a proportion of CD4+/FoxP3+ Treg cells.

FIGS. 38 and 39 illustrate results obtained by identifying effects of GI101 and GI101w on proliferation of CD8+ T cells and NK cells.

FIGS. 40 and 41 illustrate results obtained by identifying an effect of GI101 on effector T cells.

FIG. 42 illustrates results obtained by identifying effects of mGI101 and mGI102-M61 on mouse immune cells.

FIGS. 43 and 44 illustrate results obtained by identifying an effect of GI101 on cancer cells overexpressing PD-L1.

FIGS. 45 and 46 illustrate results obtained by identifying a tumor inhibitory effect of GI101 in mouse-derived colorectal cancer cell-transplanted mice.

FIG. 47 illustrates results obtained by identifying a tumor inhibitory effect of mGI101 in mouse-derived melanoma-transplanted mice.

FIG. 48 illustrates tumor inhibition of mGI101 in mouse-derived melanoma-transplanted mice.

FIG. 49 illustrates results obtained by identifying a tumor inhibitory effect of mGI101, depending on its dose, in mouse-derived colorectal cancer cell-transplanted mice.

FIG. 50 illustrates results obtained by analyzing survival rate of mouse-derived colorectal cancer cell-transplanted mice having received mGI101.

FIG. 51 illustrates results obtained by identifying a tumor inhibitory effect of GI101 in mouse-derived colorectal cancer cell-transplanted mice.

FIG. 52 illustrates results obtained by subjecting mouse-derived colorectal cancer cell-transplanted mice to treatment with hIgG4, anti-PD-1 antibody, or GI101, and then analyzing, with FACS, CD8+ T cells, IFN-γ T cells, CD4+ T cells, and Treg cells in cancer tissues.

FIG. 53 graphically illustrates results obtained by subjecting mouse-derived colorectal cancer cell-transplanted mice to treatment with hIgG4, anti-PD-1 antibody, or GI101, and then analyzing, with FACS, CD8+ T cells, IFN-γ T cells, CD4+ T cells, and Treg cells in cancer tissues.

FIG. 54 illustrates results obtained by subjecting mouse-derived colorectal cancer cell-transplanted mice to treatment with hIgG4, anti-PD-1 antibody, or GI101, and then analyzing, with FACS, macrophages in cancer tissues.

FIG. 55 graphically illustrates results obtained by subjecting mouse-derived colorectal cancer cell-transplanted mice to treatment with hIgG4, anti-PD-1 antibody, or GI101, and then analyzing, with FACS, macrophages in cancer tissues.

FIG. 56 illustrates results obtained by subjecting mouse-derived colorectal cancer cell-transplanted mice to treatment with hIgG4, anti-PD-1 antibody, or GI101, and then analyzing, with FACS, dendritic cells in cancer tissues.

FIG. 57 graphically illustrates results obtained by subjecting mouse-derived colorectal cancer cell-transplanted mice to treatment with hIgG4, anti-PD-1 antibody, or GI101, and then analyzing, with FACS, dendritic cells in cancer tissues.

FIG. 58 illustrates results obtained by identifying a tumor inhibitory effect of GI101 in mouse-derived lung cancer cell-transplanted mice.

FIG. 59 graphically illustrates results obtained by subjecting mouse-derived lung cancer cell-transplanted mice to treatment with hIgG4, anti-PD-1 antibody, or GI101, and then analyzing, with FACS, CD8+ T cells, IFN-γ T cells, CD4+ T cells, and Treg cells in cancer tissues.

FIG. 60 graphically illustrates results obtained by subjecting mouse-derived lung cancer cell-transplanted mice to treatment with hIgG4, anti-PD-1 antibody, or GI101, and then analyzing, with FACS, macrophages in cancer tissues.

FIG. 61 graphically illustrates results obtained by subjecting mouse-derived lung cancer cell-transplanted mice to treatment with hIgG4, anti-PD-1 antibody, or GI101, and then analyzing, with FACS, dendritic cells in cancer tissues.

FIG. 62 illustrates results obtained by identifying a tumor inhibitory effect of mGI102-M61 in mouse-derived colorectal cancer cell-transplanted mice.

FIG. 63 illustrates results obtained by analyzing survival rate of mouse-derived colorectal cancer cell-transplanted mice having received mGI102-M61.

FIG. 64 illustrates results obtained by identifying a tumor inhibitory effect of mGI101 in mouse-derived colorectal cancer cell-transplanted mice.

FIG. 65 illustrates tumor inhibition of mGI101 in mouse-derived colorectal cancer cell-transplanted mice.

FIG. 66 illustrates results obtained by making 15-day clinical observations for monkeys having received PBS or GI101.

FIGS. 67 and 68 illustrate results obtained by measuring body weights on days −1, 1, 8, and 15 for monkeys having received PBS or GI101.

FIG. 69 illustrates 15-day food consumption for monkeys having received PBS or GI101.

FIGS. 70 to 72 illustrate results obtained by analyzing the blood on days −1, 1, 8, and 15 for monkeys having received PBS or GI101.

FIGS. 73 to 79 illustrate results obtained by performing clinical and chemical analysis on days −1, 1, 8, and 15 days for monkeys having received PBS or GI101.

FIGS. 80 and 81 illustrate results obtained by analyzing cytokines on days −1, 1, 8, and 15 for monkeys having received PBS or GI101.

FIGS. 82 to 87 illustrate results obtained by analyzing immune cells on days −1, 1, 8, and 15 for monkeys having received PBS or GI101.

FIG. 88 illustrates results obtained by sacrificing, on day 16, monkeys having received PBS or GI101 to obtain spleen tissues, and pathologically analyzing the spleen tissues.

FIG. 89A and FIG. 89B illustrate fusion proteins, in each of which CD80 protein and IL-2 protein are bound to a carrier protein. Specifically, FIG. 89A illustrates the fusion protein in which the CD80 protein and the IL-2 protein are bound to N-terminus and C-terminus of the carrier protein, respectively. In addition, FIG. 89B illustrates the fusion protein in which the CD80 protein and the IL-2 protein are bound to C-terminus and N-terminus of the carrier protein, respectively.

FIG. 90A illustrates results obtained by measuring the number of lymphocytes on days 1, 3, 6, 9, 11, 15, 22, 24, 27, 30, 32 and 36 for monkeys having administered vehicle, GI101_SA (7.7), GI101_SA (15.37), GI101_SA (19.8) at a dose of 1 mg/kg and Proleukin at a dose of 0.1 mg/kg, respectively, on days 1 and 22.

FIG. 90B illustrates results obtained by measuring the number of lymphocytes on days 6 and 27 for monkeys having administered vehicle, GI101_SA (7.7), GI101_SA (15.37), GI101_SA (19.8) at a dose of 1 mg/kg and Proleukin at a dose of 0.1 mg/kg, respectively, on days 1 and 22.

FIG. 90C illustrates results obtained by measuring the number of lymphocytes on days 1, 3, 6, 9, 11, 15, 22, 24, 27, 30, 32 and 36 for monkeys having administered vehicle, GI101_SA (7.7), GI101_SA (15.37), GI101_SA (19.8) at a dose of 2.5 mg/kg and Proleukin at a dose of 0.1 mg/kg, respectively, on days 1 and 22.

FIG. 90D illustrates results obtained by measuring the number of lymphocytes on days 6 and 27 for monkeys having administered vehicle, GI101_SA (7.7), GI101_SA (15.37), GI101_SA (19.8) at a dose of 2.5 mg/kg and Proleukin at a dose of 0.1 mg/kg, respectively, on days 1 and 22.

FIG. 91A illustrates results obtained by measuring the number of CD8+ T cells on days 1, 3, 6, 9, 11, 15, 22, 24, 27, 30, 32 and 36 for monkeys having administered vehicle, GI101_SA (7.7), GI101_SA (15.37), GI101_SA (19.8) at a dose of 1 mg/kg and Proleukin at a dose of 0.1 mg/kg, respectively, on days 1 and 22.

FIG. 91B illustrates results obtained by measuring the number of CD8+ T cells on days 6 and 27 for monkeys having administered vehicle, GI101_SA (7.7), GI101_SA (15.37), GI101_SA (19.8) at a dose of 1 mg/kg and Proleukin at a dose of 0.1 mg/kg, respectively, on days 1 and 22.

FIG. 91C illustrates results obtained by measuring the number of CD8+ T cells on days 1, 3, 6, 9, 11, 15, 22, 24, 27, 30, 32 and 36 for monkeys having administered vehicle, GI101_SA (7.7), GI101_SA (15.37), GI101_SA (19.8) at a dose of 2.5 mg/kg and Proleukin at a dose of 0.1 mg/kg, respectively, on days 1 and 22.

FIG. 91D illustrates results obtained by measuring the number of CD8+ T cells on days 6 and 27 for monkeys having administered vehicle, GI101_SA (7.7), GI101_SA (15.37), GI101_SA (19.8) at a dose of 2.5 mg/kg and Proleukin at a dose of 0.1 mg/kg, respectively, on days 1 and 22.

FIG. 92A illustrates results obtained by measuring the number of NK cells on days 1, 3, 6, 9, 11, 15, 22, 24, 27, 30, 32 and 36 for monkeys having administered vehicle, GI101_SA (7.7), GI101_SA (15.37), GI101_SA (19.8) at a dose of 1 mg/kg and Proleukin at a dose of 0.1 mg/kg, respectively, on days 1 and 22.

FIG. 92B illustrates results obtained by measuring the number of NK cells on days 6 and 27 for monkeys having administered vehicle, GI101_SA (7.7), GI101_SA (15.37), GI101_SA (19.8) at a dose of 1 mg/kg and Proleukin at a dose of 0.1 mg/kg, respectively, on days 1 and 22.

FIG. 92C illustrates results obtained by measuring the number of NK cells on days 1, 3, 6, 9, 11, 15, 22, 24, 27, 30, 32 and 36 for monkeys having administered vehicle, GI101_SA (7.7), GI101_SA (15.37), GI101_SA (19.8) at a dose of 2.5 mg/kg and Proleukin at a dose of 0.1 mg/kg, respectively, on days 1 and 22.

FIG. 92D illustrates results obtained by measuring the number of NK cells on days 6 and 27 for monkeys having administered vehicle, GI101_SA (7.7), GI101_SA (15.37), GI101_SA (19.8) at a dose of 2.5 mg/kg and Proleukin at a dose of 0.1 mg/kg, respectively, on days 1 and 22.

FIG. 93A illustrates results obtained by measuring the number of lymphocytes on days 1, 3, 6, 9, 11, 15, 22, 24, 27, 30, 32 and 36 for monkeys having administered vehicle, GI102_SA (19) at a dose of 1 mg/kg and Proleukin at a dose of 0.1 mg/kg, respectively, on days 1 and 22.

FIG. 93B illustrates results obtained by measuring the number of lymphocytes on days 6 and 27 for monkeys having administered vehicle, GI102_SA (19) at a dose of 1 mg/kg and Proleukin at a dose of 0.1 mg/kg, respectively, on days 1 and 22.

FIG. 93C illustrates results obtained by measuring the number of lymphocytes on days 1, 3, 6, 9, 11, 15, 22, 24, 27, 30, 32 and 36 for monkeys having administered vehicle, GI102_SA (19) at a dose of 2.5 mg/kg and Proleukin at a dose of 0.1 mg/kg, respectively, on days 1 and 22.

FIG. 93D illustrates results obtained by measuring the number of lymphocytes on days 6 and 27 for monkeys having administered vehicle, GI102_SA (19) at a dose of 2.5 mg/kg and Proleukin at a dose of 0.1 mg/kg, respectively, on days 1 and 22.

FIG. 94A illustrates results obtained by measuring the number of CD8+ T cells on days 1, 3, 6, 9, 11, 15, 22, 24, 27, 30, 32 and 36 for monkeys having administered vehicle, GI102_SA (19) at a dose of 1 mg/kg and Proleukin at a dose of 0.1 mg/kg, respectively, on days 1 and 22.

FIG. 94B illustrates results obtained by measuring the number of CD8+ T cells on days 6 and 27 for monkeys having administered vehicle, GI102_SA (19) at a dose of 1 mg/kg and Proleukin at a dose of 0.1 mg/kg, respectively, on days 1 and 22.

FIG. 94C illustrates results obtained by measuring the number of CD8+ T cells on days 1, 3, 6, 9, 11, 15, 22, 24, 27, 30, 32 and 36 for monkeys having administered vehicle, GI102_SA (19) at a dose of 2.5 mg/kg and Proleukin at a dose of 0.1 mg/kg, respectively, on days 1 and 22.

FIG. 94D illustrates results obtained by measuring the number of CD8+ T cells on days 6 and 27 for monkeys having administered vehicle, GI102_SA (19) at a dose of 2.5 mg/kg and Proleukin at a dose of 0.1 mg/kg, respectively, on days 1 and 22.

FIG. 95A illustrates results obtained by measuring the number of NK cells on days 1, 3, 6, 9, 11, 15, 22, 24, 27, 30, 32 and 36 for monkeys having administered vehicle, GI102_SA (19) at a dose of 1 mg/kg and Proleukin at a dose of 0.1 mg/kg, respectively, on days 1 and 22.

FIG. 95B illustrates results obtained by measuring the number of NK cells on days 6 and 27 for monkeys having administered vehicle, GI102_SA (19) at a dose of 1 mg/kg and Proleukin at a dose of 0.1 mg/kg, respectively, on days 1 and 22.

FIG. 95C illustrates results obtained by measuring the number of NK cells on days 1, 3, 6, 9, 11, 15, 22, 24, 27, 30, 32 and 36 for monkeys having administered vehicle, GI102_SA (19) at a dose of 2.5 mg/kg and Proleukin at a dose of 0.1 mg/kg, respectively, on days 1 and 22.

FIG. 95D illustrates results obtained by measuring the number of NK cells on days 6 and 27 for monkeys having administered vehicle, GI102_SA (19) at a dose of 2.5 mg/kg and Proleukin at a dose of 0.1 mg/kg, respectively, on days 1 and 22.

FIG. 96A illustrates results obtained by measuring the number of lymphocytes up to on day 21 for monkeys having administered GI102_SA (25) at a dose of 0.1 mg/kg, 0.3 mg/kg and 1.0 mg/kg, respectively, on day 1.

FIG. 96B illustrates results obtained by measuring the number of CD8+ T cells up to on day 21 for monkeys having administered GI102_SA (25) at a dose of 0.1 mg/kg, 0.3 mg/kg and 1.0 mg/kg, respectively, on day 1.

FIG. 96C illustrates results obtained by measuring the number of NK cells up to on day 21 for monkeys having administered GI102_SA (25) at a dose of 0.1 mg/kg, 0.3 mg/kg and 1.0 mg/kg, respectively, on day 1.

FIG. 97A illustrates results obtained by measuring the number of lymphocytes on days 0 (pre-dose), 7, 9 and 15 for monkeys having administered GI102_SA (25) at a dose of 0.1 mg/kg, 0.3 mg/kg and 1.0 mg/kg, respectively, on day 1.

FIG. 97B illustrates results obtained by measuring the number of CD8+ T cells on days 0 (pre-dose), 7, 9 and 15 for monkeys having administered GI102_SA (25) at a dose of 0.1 mg/kg, 0.3 mg/kg and 1.0 mg/kg, respectively, on day 1.

FIG. 97C illustrates results obtained by measuring the number of NK cells on days 0 (pre-dose), 7, 9 and 15 for monkeys having administered GI102_SA (25) at a dose of 0.1 mg/kg, 0.3 mg/kg and 1.0 mg/kg, respectively, on day 1.

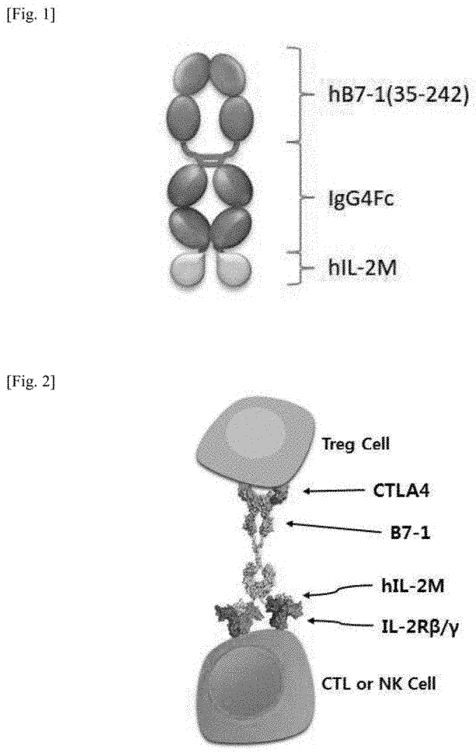

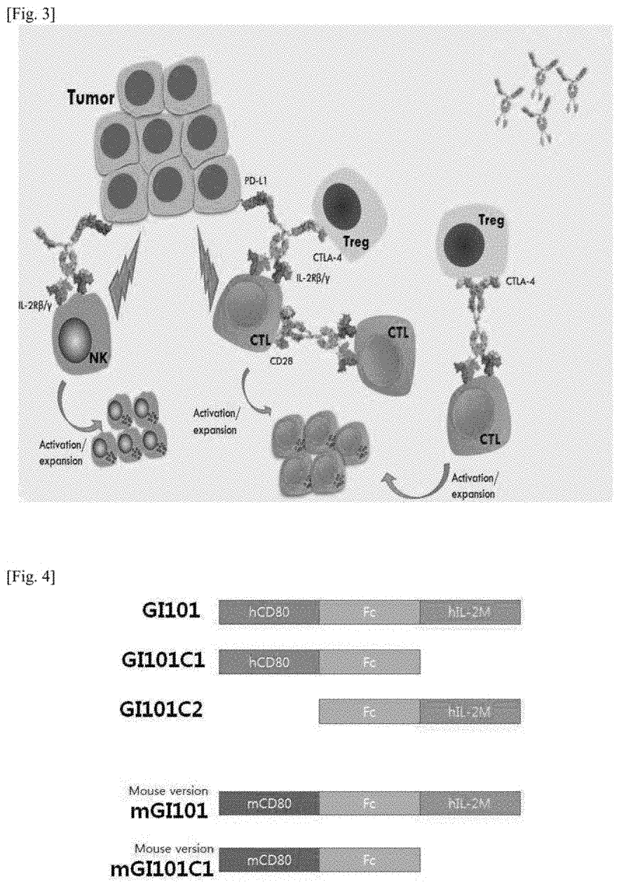

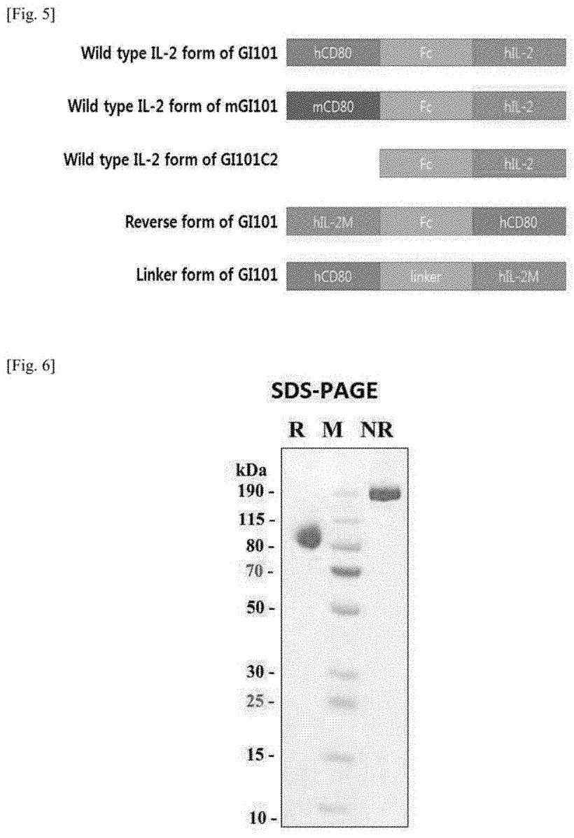

### BEST MODE

Fusion Protein Comprising IL-2 Protein and CD80 Protein

In an aspect of the present invention, there is provided a fusion protein comprising an IL-2 protein and a CD80 protein.

As used herein, the term “IL-2” or “interleukin-2”, unless otherwise stated, refers to any wild-type IL-2 obtained from any vertebrate source, including mammals, for example, primates (such as humans) and rodents (such as mice and rats). IL-2 may be obtained from animal cells, and also includes one obtained from recombinant cells capable of producing IL-2. In addition, IL-2 may be wild-type IL-2 or a variant thereof.

In the present specification, IL-2 or a variant thereof may be collectively expressed by the term “IL-2 protein” or “IL-2 polypeptide.” IL-2, an IL-2 protein, an IL-2 polypeptide, and an IL-2 variant specifically bind to, for example, an IL-2 receptor. This specific binding may be identified by methods known to those skilled in the art.

An embodiment of IL-2 may have the amino acid sequence of SEQ ID NO: 35 or SEQ ID NO: 36. Here, IL-2 may also be in a mature form. Specifically, the mature IL-2 may not contain a signal sequence, and may have the amino acid sequence of SEQ ID NO: 10. Here, IL-2 may be used under a concept encompassing a fragment of wild-type IL-2 in which a portion of N-terminus or C-terminus of the wild-type IL-2 is truncated.

In addition, the fragment of IL-2 may be in a form in which 1, 2, 3, 4, 5, 6, 7, 8, 9, 10, 11, 12, 13, 14, 15, 16, 17, 18, 19, 20, 21, 22, 23, 24, or 25 contiguous amino acids are truncated from N-terminus of a protein having the amino acid sequence of SEQ ID NO: 35 or SEQ ID NO: 36. In addition, the fragment of IL-2 may be in a form in which 1, 2, 3, 4, 5, 6, 7, 8, 9, 10, 11, 12, 13, 14, 15, 16, 17, 18, 19, 20, 21, 22, 23, 24, or 25 contiguous amino acids are truncated from C-terminus of a protein having the amino acid sequence of SEQ ID NO: 35 or SEQ ID NO: 36.

As used herein, the term “IL-2 variant” refers to a form in which a portion of amino acids in the full-length IL-2 or the above-described fragment of IL-2 is substituted. That is, an IL-2 variant may have an amino acid sequence different from wild-type IL-2 or a fragment thereof. However, an IL-2 variant may have activity equivalent or similar to the wild-type IL-2. Here, “IL-2 activity” may, for example, refer to specific binding to an IL-2 receptor, which specific binding can be measured by methods known to those skilled in the art.

Specifically, an IL-2 variant may be obtained by substitution of a portion of amino acids in the wild-type IL-2. An embodiment of the IL-2 variant obtained by amino acid substitution may be obtained by substitution of at least one of the 38^{th}, 42^{nd}, 45^{th}, 61^{st}, and 72^{nd} amino acids in the amino acid sequence of SEQ ID NO: 10.

Specifically, the IL-2 variant may be obtained by substitution of at least one of the 38^{th}, 42^{nd}, 45^{th}, 61^{st}, or 72^{nd} amino acid in the amino acid sequence of SEQ ID NO: 10 with another amino acid. In addition, when IL-2 is in a form in which a portion of N-terminus in the amino acid sequence of SEQ ID NO: 35 is truncated, the amino acid at a position complementarily corresponding to that in the amino acid sequence of SEQ ID NO: 10 may be substituted with another amino acid. For example, when IL-2 has the amino acid sequence of SEQ ID NO: 35, its IL-2 variant may be obtained by substitution of at least one of 58^{th}, 62^{nd}, 65^{th}, 81^{st}, or 92^{nd} amino acid in the amino acid sequence of SEQ ID NO: 35 with another amino acid. These amino acid residues correspond to the 38^{th}, 42^{nd}, 45^{th}, 61^{st}, and 72^{nd} amino acid residues in the amino acid sequence of SEQ ID NO: 10, respectively. According to an embodiment, one, two, three, four, five, six, seven, eight, nine, or ten amino acids may be substituted as long as such IL-2 variant maintains IL-2 activity. According to another embodiment, one to five amino acids may be substituted.

In an embodiment, an IL-2 variant may be in a form in which two amino acids are substituted. Specifically, the IL-2 variant may be obtained by substitution of the 38^{th} and 42^{nd} amino acids in the amino acid sequence of SEQ ID NO: 10. In addition, in an embodiment, the IL-2 variant may be obtained by substitution of the 38^{th} and 45^{th} amino acids in the amino acid sequence of SEQ ID NO: 10. In addition, in an embodiment, the IL-2 variant may be obtained by substitution of the 38^{th} and 61^{st} amino acids in the amino acid sequence of SEQ ID NO: 10. In addition, in an embodiment, the IL-2 variant may be obtained by substitution of the 38^{th} and 72^{nd} amino acids in the amino acid sequence of SEQ ID NO: 10. In addition, in an embodiment, the IL-2 variant may be obtained by substitution of the 42^{nd} and 45^{th} amino acids in the amino acid sequence of SEQ ID NO: 10. In addition, in an embodiment, the IL-2 variant may be obtained by substitution of the 42^{nd} and 61^{st} amino acids in the amino acid sequence of SEQ ID NO: 10. In addition, in an embodiment, the IL-2 variant may be obtained by substitution of the 42^{nd} and 72^{nd} amino acids in the amino acid sequence of SEQ ID NO: 10. In addition, in an embodiment, the IL-2 variant may be obtained by substitution of the 45^{th} and 61^{st} amino acids in the amino acid sequence of SEQ ID NO: 10. In addition, in an embodiment, the IL-2 variant may be obtained by substitution of the 45^{th} and 72^{nd} amino acids in the amino acid sequence of SEQ ID NO: 10. In addition, in an embodiment, the IL-2 variant may be obtained by substitution of the 61^{st} and 72^{nd} amino acids in the amino acid sequence of SEQ ID NO: 10.

Furthermore, an IL-2 variant may be in a form in which three amino acids are substituted. Specifically, the IL-2 variant may be obtained by substitution of the 38^{th}, 42^{nd}, and 45^{th} amino acids in the amino acid sequence of SEQ ID NO: 10. In addition, in an embodiment, the IL-2 variant may be obtained by substitution of the 38^{th}, 42^{nd}, and 61^{st} amino acids in the amino acid sequence of SEQ ID NO: 10. In addition, in an embodiment, the IL-2 variant may be obtained by substitution of the 38^{th}, 42^{nd}, and 72^{nd} amino acids in the amino acid sequence of SEQ ID NO: 10. In addition, in an embodiment, the IL-2 variant may be obtained by substitution of the 38^{th}, 45^{th}, and 61^{st} amino acids in the amino acid sequence of SEQ ID NO: 10. In addition, in an embodiment, the IL-2 variant may be obtained by substitution of the 38^{th}, 45^{th}, and 72^{nd} amino acids in the amino acid sequence of SEQ ID NO: 10. In addition, in an embodiment, the IL-2 variant may be obtained by substitution of the 38^{th}, 61^{st}, and 72^{nd} amino acids in the amino acid sequence of SEQ ID NO: 10. In addition, in an embodiment, the IL-2 variant may be obtained by substitution of the 42^{nd}, 45^{th}, and 61^{st} amino acids in the amino acid sequence of SEQ ID NO: 10. In addition, in an embodiment, the IL-2 variant may be obtained by substitution of the 42^{nd}, 45^{th}, and 72^{nd} amino acids in the amino acid sequence of SEQ ID NO: 10. In addition, in an embodiment, the IL-2 variant may be obtained by substitution of the 45^{th}, 61^{st}, and 72^{nd} amino acids in the amino acid sequence of SEQ ID NO: 10.

In addition, an IL-2 variant may be in a form in which four amino acids are substituted. Specifically, the IL-2 variant may be obtained by substitution of the 38^{th}, 42^{nd}, 45^{th}, and 61^{st} amino acids in the amino acid sequence of SEQ ID NO: 10. In addition, in an embodiment, the IL-2 variant may be obtained by substitution of the 38^{th}, 42^{nd}, 45^{th}, and 72^{nd} amino acids in the amino acid sequence of SEQ ID NO: 10. In addition, in an embodiment, the IL-2 variant may be obtained by substitution of the 38^{th}, 45^{th}, 61^{st}, and 72^{nd} amino acids in the amino acid sequence of SEQ ID NO: 10. In addition, in an embodiment, the IL-2 variant may be obtained by substitution of the 38^{th}, 42^{nd}, 61^{st}, and 72^{nd} amino acids in the amino acid sequence of SEQ ID NO: 10. In addition, in an embodiment, the IL-2 variant may be obtained by substitution of 42^{nd}, 45^{th}, 61^{st}, and 72^{nd} amino acids in the amino acid sequence of SEQ ID NO: 10.

Furthermore, an IL-2 variant may be in a form in which five amino acids are substituted. Specifically, the IL-2 variant may be obtained by substitution of each of the 38^{th}, 42^{nd}, 45^{th}, 61^{st}, and 72^{nd} amino acids in the amino acid sequence of SEQ ID NO: 10 with another amino acid.

Here, the “another amino acid” introduced by the substitution may be any one selected from the group consisting of alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine. However, regarding amino acid substitution for the IL-2 variant, in the amino acid sequence of SEQ ID NO: 10, the 38^{th} amino acid cannot be substituted with arginine, the 42^{nd} amino acid cannot be substituted with phenylalanine, the 45^{th} amino acid cannot be substituted with tyrosine, the 61^{st} amino acid cannot be substituted with glutamic acid, and the 72^{nd} amino acid cannot be substituted with leucine.

Regarding amino acid substitution for an IL-2 variant, in the amino acid sequence of SEQ ID NO: 10, the 38^{th} amino acid, arginine, may be substituted with an amino acid other than arginine. Preferably, regarding amino acid substitution for an IL-2 variant, in the amino acid sequence of SEQ ID NO: 10, the 38^{th} amino acid, arginine, may be substituted with alanine (R38A).

Regarding amino acid substitution for an IL-2 variant, in the amino acid sequence of SEQ ID NO: 10, the 42^{nd} amino acid, phenylalanine, may be substituted with an amino acid other than phenylalanine. Preferably, regarding amino acid substitution for an IL-2 variant, in the amino acid sequence of SEQ ID NO: 10, the 42^{nd} amino acid, phenylalanine, may be substituted with alanine (F42A).

Regarding amino acid substitution for an IL-2 variant, in the amino acid sequence of SEQ ID NO: 10, the 45^{th} amino acid, tyrosine, may be substituted with an amino acid other than tyrosine. Preferably, regarding amino acid substitution for an IL-2 variant, in the amino acid sequence of SEQ ID NO: 10, the 45^{th} amino acid, tyrosine, may be substituted with alanine (Y45A).

Regarding amino acid substitution for an IL-2 variant, in the amino acid sequence of SEQ ID NO: 10, the 61^{st} amino acid, glutamic acid, may be substituted with an amino acid other than glutamic acid. Preferably, regarding amino acid substitution for an IL-2 variant, in the amino acid sequence of SEQ ID NO: 10, the 61^{st} amino acid, glutamic acid, may be substituted with arginine (E61R).

Regarding amino acid substitution for an IL-2 variant, in the amino acid sequence of SEQ ID NO: 10, the 72^{nd} amino acid, leucine, may be substituted with an amino acid other than leucine. Preferably, regarding amino acid substitution for an IL-2 variant, in the amino acid sequence of SEQ ID NO: 10, the 72^{nd} amino acid, leucine, may be substituted with glycine (L72G).

Specifically, an IL-2 variant may be obtained by at least one substitution selected from the group consisting of R38A, F42A, Y45A, E61R, and L72G, in the amino acid sequence of SEQ ID NO: 10.

Specifically, an IL-2 variant may be obtained by amino acid substitutions at two, three, four, or five positions among the positions selected from the group consisting of R38A, F42A, Y45A, E61R, and L72G.

In addition, an IL-2 variant may be in a form in which two amino acids are substituted. Specifically, an IL-2 variant may be obtained by the substitutions, R38A and F42A. In addition, in an embodiment, an IL-2 variant may be obtained by the substitutions, R38A and Y45A. In addition, in an embodiment, an IL-2 variant may be obtained by the substitutions, R38A and E61R. In addition, in an embodiment, an IL-2 variant may be obtained by the substitutions, R38A and L72G. In addition, in an embodiment, an IL-2 variant may be obtained by the substitutions, F42A and Y45A. In addition, in an embodiment, an IL-2 variant may be obtained by the substitutions, F42A and E61R. In addition, in an embodiment, an IL-2 variant may be obtained by the substitutions, F42A and L72G. In addition, in an embodiment, an IL-2 variant may be obtained by the substitutions, E61R and L72G.

Furthermore, an IL-2 variant may be in a form in which three amino acids are substituted. Specifically, an IL-2 variant may be obtained by the substitutions, R38A, F42A, and Y45A. In addition, in an embodiment, an IL-2 variant may be obtained by the substitutions, R38A, F42A, and E61R. In addition, in an embodiment, an IL-2 variant may be obtained by the substitutions, R38A, F42A, and L72G. In addition, in an embodiment, an IL-2 variant may be obtained by the substitutions, R38A, Y45A, and E61R. In addition, in an embodiment, an IL-2 variant may be obtained by the substitutions, R38A, Y45A, and L72G. In addition, in an embodiment, an IL-2 variant may be obtained by the substitutions, F42A, Y45A, and E61R. In addition, in an embodiment, an IL-2 variant may be obtained by the substitutions, F42A, Y45A, and L72G. In addition, in an embodiment, an IL-2 variant may be obtained by the substitutions, F42A, E61R, and L72G. In addition, in an embodiment, an IL-2 variant may be obtained by the substitutions, Y45A, E61R, and L72G.

In addition, an IL-2 variant may be in a form in which four amino acids are substituted. Specifically, an IL-2 variant may be obtained by the substitutions, R38A, F42A, Y45A, and E61R. In addition, in an embodiment, an IL-2 variant may be obtained by the substitutions, R38A, F42A, Y45A, and L72G. In addition, in an embodiment, an IL-2 variant may be obtained by the substitutions, R38A, F42A, E61R, and L72G. In addition, in an embodiment, an IL-2 variant may be obtained by the substitutions, R38A, Y45A, E61R, and L72G. In addition, in an embodiment, an IL-2 variant may be obtained by the substitutions, F42A, Y45A, E61R, and L72G.

Furthermore, an IL-2 variant may be obtained by the substitutions, R38A, F42A, Y45A, E61R, and L72G.

Preferably, an embodiment of the IL-2 variant may contain which are any one selected from the following substitution combinations (a) to (d) in the amino acid sequence of SEQ ID NO: 10:

(a) R38A/F42A

(b) R38A/F42A/Y45A

(c) R38A/F42A/E61R

(d) R38A/F42A/L72G

Here, when IL-2 has the amino acid sequence of SEQ ID NO: 35, an amino acid substitution may be present at a position complementarily corresponding to that in the amino acid sequence of SEQ ID NO: 10. In addition, even when IL-2 is a fragment of the amino acid sequence of SEQ ID NO: 35, an amino acid substitution may be present at a position complementarily corresponding to that in the amino acid sequence of SEQ ID NO: 10.

Specifically, an IL-2 variant may have the amino acid sequence of SEQ ID NO: 6, 22, 23, or 24.

In addition, an IL-2 variant may be characterized by having low in vivo toxicity. Here, the low in vivo toxicity may be a side effect caused by binding of IL-2 to the IL-2 receptor alpha chain (IL-2Rα). Various IL-2 variants have been developed to ameliorate the side effect caused by binding of IL-2 to IL-2Rα, and such IL-2 variants may be those disclosed in U.S. Pat. No. 5,229,109 and Korean Patent No. 1667096. In particular, IL-2 variants described in the present application have low binding ability for the IL-2 receptor alpha chain (IL-2Rα) and thus have lower in vivo toxicity than the wild-type IL-2.

As used herein, the term “CD80”, also called “B7-1”, is a membrane protein present in dendritic cells, activated B cells, and monocytes. CD80 provides co-stimulatory signals essential for activation and survival of T cells. CD80 is known as a ligand for the two different proteins, CD28 and CTLA-4, present on the surface of T cells. CD80 is composed of 288 amino acids, and may specifically have the amino acid sequence of SEQ ID NO: 11. In addition, as used herein, the term “CD80 protein” refers to the full-length CD80 or a CD80 fragment.

As used herein, the term “CD80 fragment” refers to a cleaved form of CD80. In addition, the CD80 fragment may be an extracellular domain of CD80. An embodiment of the CD80 fragment may be obtained by elimination of the 1^{st} to 34^{th} amino acids from N-terminus which are a signal sequence of CD80. Specifically, an embodiment of the CD80 fragment may be a protein composed of the 35^{th} to 288^{th} amino acids in SEQ ID NO: 11. In addition, an embodiment of the CD80 fragment may be a protein composed of the 35^{th} to 242^{nd} amino acids in SEQ ID NO: 11. In addition, an embodiment of the CD80 fragment may be a protein composed of the 35^{th} to 232^{nd} amino acids in SEQ ID NO: 11. In addition, an embodiment of the CD80 fragment may be a protein composed of the 35^{th} to 139^{th} amino acids in SEQ ID NO: 11. In addition, an embodiment of the CD80 fragment may be a protein composed of the 142^{nd} to 242^{nd} amino acids in SEQ ID NO: 11. In an embodiment, a CD80 fragment may have the amino acid sequence of SEQ ID NO: 2.

In addition, the IL-2 protein and the CD80 protein may be attached to each other via a linker or a carrier. Specifically, the IL-2 or a variant thereof and the CD80 (B7-1) or a fragment thereof may be attached to each other via a linker or a carrier. In the present description, the linker and the carrier may be used interchangeably.

The linker links two proteins. An embodiment of the linker may include 1 to 50 amino acids, albumin or a fragment thereof, an Fc domain of an immunoglobulin, or the like. Here, the Fc domain of immunoglobulin refers to a protein that contains heavy chain constant region 2 (CH2) and heavy chain constant region 3 (CH3) of an immunoglobulin, and does not contain heavy and light chain variable regions and light chain constant region 1 (CH1) of an immunoglobulin. The immunoglobulin may be IgG, IgA, IgE, IgD, or IgM, and may preferably be IgG4. Here, Fc domain of wild-type immunoglobulin G4 may have the amino acid sequence of SEQ ID NO: 4.

In addition, the Fc domain of an immunoglobulin may be an Fc domain variant as well as wild-type Fc domain. In addition, as used herein, the term “Fc domain variant” may refer to a form which is different from the wild-type Fc domain in terms of glycosylation pattern, has a high glycosylation as compared with the wild-type Fc domain, or has a low glycosylation as compared with the wild-type Fc domain, or a deglycosylated form. In addition, an aglycosylated Fc domain is included therein. The Fc domain or a variant thereof may be adapted to have an adjusted number of sialic acids, fucosylations, or glycosylations, through culture conditions or genetic manipulation of a host.

In addition, glycosylation of the Fc domain of an immunoglobulin may be modified by conventional methods such as chemical methods, enzymatic methods, and genetic engineering methods using microorganisms. In addition, the Fc domain variant may be in a mixed form of respective Fc regions of immunoglobulins, IgG, IgA, IgE, IgD, and IgM. In addition, the Fc domain variant may be in a form in which some amino acids of the Fc domain are substituted with other amino acids. An embodiment of the Fc domain variant may have the amino acid sequence of SEQ ID NO: 12.

The fusion protein may have a structure in which, using an Fc domain as a linker (or carrier), a CD80 protein and an IL-2 protein, or an IL-2 protein and a CD80 protein are linked to N-terminus and C-terminus of the linker or carrier, respectively (FIG. 89). Linkage between N-terminus or C-terminus of the Fc domain and CD-80 or IL-2 may optionally be achieved by a linker peptide.

Specifically, a fusion protein may consist of the following structural formula (I) or (II):

N′-X-[linker (1)]ₙ-Fc domain-[linker (2)]ₘ-Y-C′ (I)

N′-Y-[linker (1)]ₙ-Fc domain-[linker (2)]ₘ-X-C′ (II)

Here, in the structural formulas (I) and (II),

N′ is the N-terminus of the fusion protein,

C′ is the C-terminus of the fusion protein,

X is a CD80 protein,

Y is an IL-2 protein,

the linkers (1) and (2) are peptide linkers, and

n and m are each independently 0 or 1.

Preferably, the fusion protein may consist of the structural formula (I). The IL-2 protein is as described above. In addition, the CD80 protein is as described above. According to an embodiment, the IL-2 protein may be an IL-2 variant with one to five amino acid substitutions as compared with the wild-type IL-2. The CD80 protein may be a fragment obtained by truncation of up to about 34 contiguous amino acid residues from the N-terminus or C-terminus of the wild-type CD80. Alternatively, the CD protein may be an extracellular immunoglobulin-like domain having the activity of binding to the T cell surface receptors CTLA-4 and CD28.

Specifically, the fusion protein may have the amino acid sequence of SEQ ID NO: 9, 26, 28, or 30. According to another embodiment, the fusion protein (monomer) includes a polypeptide having a sequence identity of 85%, 86%, 87%, 88%, 89%, 90%, 91%, 92%, 93%, 94%, 95%, 96%, 97%, 98%, 99%, or 100% to the amino acid sequence of SEQ ID NO: 9, 26, 28, or 30. Here, the identity is, for example, percent homology, and may be determined through homology comparison software such as BlastN software of the National Center of Biotechnology Information (NCBI).

The peptide linker (1) may be included between the CD80 protein and the Fc domain. The peptide linker (1) may consist of 5 to 80 contiguous amino acids, 20 to 60 contiguous amino acids, 25 to 50 contiguous amino acids, or 30 to 40 contiguous amino acids. In an embodiment, the peptide linker (1) may consist of 30 amino acids. In addition, the peptide linker (1) may contain at least one cysteine. Specifically, the peptide linker (1) may contain one, two, or three cysteines. In addition, the peptide linker (1) may be derived from the hinge of an immunoglobulin. In an embodiment, the peptide linker (1) may be a peptide linker consisting of the amino acid sequence of SEQ ID NO: 3.

The peptide linker (2) may consist of 1 to 50 contiguous amino acids, 3 to 30 contiguous amino acids, or 5 to 15 contiguous amino acids. In an embodiment, the peptide linker (2) may be (G4S)ₙ (where n is an integer of 1 to 10). Here, in (G4S)ₙ, n may be 1, 2, 3, 4, 5, 6, 7, 8, 9, or 10. In an embodiment, the peptide linker (2) may be a peptide linker consisting of the amino acid sequence of SEQ ID NO: 5.

In another aspect of the present invention, there is provided a dimer obtained by binding of two fusion proteins, each of which comprises an IL-2 protein and a CD80 protein and having a high content of sialic acid. The fusion protein comprising IL-2 or a variant thereof and CD80 or a fragment thereof is as described above.

Here, the binding between the fusion proteins constituting the dimer may be achieved by, but is not limited to, a disulfide bond formed by cysteines present in the linker. The fusion proteins constituting the dimer may be the same or different fusion proteins from each other. Preferably, the dimer may be a homodimer. An embodiment of the fusion protein constituting the dimer may be a protein having the amino acid sequence of SEQ ID NO: 9.

The term “sialic acid” used in the present invention may include N-acetylneuraminic acid (Neu5Ac) of Formula (III) below and N-glycolylneuraminic acid (Neu5Gc) of Formula (IV) below.

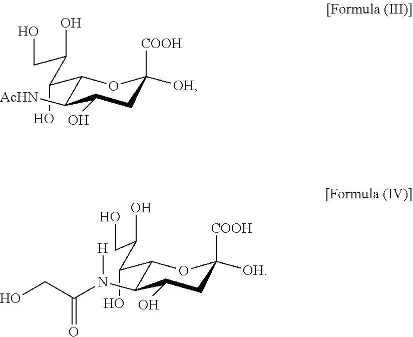

Specifically, the sialic acid may be N-acetylneuraminic acid (Neu5Ac).

In addition, the content of sialic acid of the fusion protein dimer may be increased by producing a fusion protein in a cell into which a sialic acid transferase gene is introduced. In addition, the sialic acid content of the fusion protein dimer may be increased by adjusting the culture days or the time of the recovery process of the culture medium.

In an embodiment, the molar ratio of sialic acid to the fusion protein dimer may be 7 or more, for example, the molar ratio of sialic acid to the fusion protein dimer may be 7, 8, 9, 10, 11, 12, 13, 14, 15, 16, 17, 18, 19, 20, 21, 22, 23, 24, 25, 26, 27, 28, 29 or 30. Preferably, the molar ratio of sialic acid to the fusion protein dimer may be 7, 8, 9, 10, 11, 12, 13, 14, 15, 16, 17, 18, 19, 20, 21, 22, 23, 24 or 25.

In an embodiment, the molar ratio of sialic acid to the fusion protein dimer may be at least 7, at least 8, at least 9, at least 10, at least 11, at least 12, at least 13, at least 14, at least 15, at least 16, at least 17, at least 18, at least 19, at least 20, or at least 21.

Specifically, the molar ratio of sialic acid to the fusion protein dimer may be from 7 to 25. More specifically, the molar ratio of sialic acid to the fusion protein dimer may be from 7 to 24, from 7 to 23, from 7 to 22, from 7 to 21, from 7 to 20, from 7 to 19, from 7 to 18, from 7 to 17, from 7 to 16, from 7 to 15, from 7 to 14, from 7 to 13, from 7 to 12, from 7 to 11, from 7 to 10, from 7 to 9, from 7 to 25, from 8 to 25, from 9 to 25, from 10 to 25, from 11 to 25, from 12 to 25, from 13 to 25, from 14 to 25, from 15 to 25, from 16 to 25, from 17 to 25, from 18 to 25, or from 19 to 25. The molar ratio of sialic acid to the fusion protein dimer may be about 7.7, about 15.37, or about 19.8.

More specifically, the molar ratio of sialic acid to the fusion protein dimer may be from 15 to 30. The molar ratio of sialic acid to the fusion protein dimer may from 15 to 29, from 15 to 28, from 15 to 27, from 15 to 26, from 15 to 25, from 16 to 30, from 17 to 30, from 18 to 30, or from 19 to 30. The molar ratio of sialic acid to the fusion protein dimer may be about 19 or about 25.

In addition, the fusion protein having a high content of sialic acid may proliferate immune cells, such as lymphocytes including CD8+ T cells and natural killer cells.

Pharmaceutical Composition Comprising the Fusion Protein Dimer

In another aspect of the present invention, there is provided a pharmaceutical composition comprising the fusion protein dimer.

The fusion protein dimer is the same as described above. In particular, the pharmaceutical composition may be characterized by being for injection. It was confirmed that a fusion protein dimer having a high content of sialic acid is effective in enhancing immunity compared to a fusion protein dimer having a low content of sialic acid upon injection.

A preferred dose of the pharmaceutical composition varies depending on the patient's condition and body weight, severity of disease, form of drug, route and duration of administration and may be appropriately selected by those skilled in the art. In the pharmaceutical composition for treating or preventing cancer or an infectious disease of the present invention, the active ingredient may be contained in any amount (effective amount) depending on application, dosage form, blending purpose, and the like, as long as the active ingredient can exhibit anticancer activity or a therapeutic effect on an infectious disease. A conventional effective amount thereof will be determined within a range of 0.001% to 20.0% by weight, based on the total weight of the composition. Here, the term “effective amount” refers to an amount of an active ingredient capable of inducing an anticancer effect or an infectious disease-treating effect. Such an effective amount can be experimentally determined within the scope of common knowledge of those skilled in the art.

As used herein, the term “treatment” may be used to mean both therapeutic and prophylactic treatment. Here, prophylaxis may be used to mean that a pathological condition or disease of an individual is alleviated or mitigated. In an embodiment, the term “treatment” includes both application or any form of administration for treating a disease in a mammal, including a human. In addition, the term includes inhibiting or slowing down a disease or disease progression; and includes meanings of restoring or repairing impaired or lost function so that a disease is partially or completely alleviated; stimulating inefficient processes; or alleviating a serious disease.

As used herein, the term “efficacy” refers to capacity that can be determined by one or parameters, for example, survival or disease-free survival over a certain period of time such as one year, five years, or ten years. In addition, the parameter may include inhibition of size of at least one tumor in an individual.

Pharmacokinetic parameters such as bioavailability and underlying parameters such as clearance rate may also affect efficacy. Thus, “enhanced efficacy” (for example, improvement in efficacy) may be due to enhanced pharmacokinetic parameters and improved efficacy, which may be measured by comparing clearance rate and tumor growth in test animals or human subjects, or by comparing parameters such as survival, recurrence, or disease-free survival.

As used herein, the term “therapeutically effective amount” or “pharmaceutically effective amount” refers to an amount of a compound or composition effective to prevent or treat the disease in question, which is sufficient to treat the disease at a reasonable benefit/risk ratio applicable to medical treatment and does not cause adverse effects. A level of the effective amount may be determined depending on factors including the patient's health condition, type and severity of disease, activity of drug, the patient's sensitivity to drug, mode of administration, time of administration, route of administration and excretion rate, duration of treatment, formulation or simultaneously used drugs, and other factors well known in the medical field. In an embodiment, the therapeutically effective amount means an amount of drug effective to treat cancer.

Here, the pharmaceutical composition may further comprise a pharmaceutically acceptable carrier. The pharmaceutically acceptable carrier may be any carrier as long as the carrier is a non-toxic substance suitable for delivery to a patient. Distilled water, alcohol, fat, wax, and inert solid may be contained as the carrier. A pharmaceutically acceptable adjuvant (buffer, dispersant) may also be contained in the pharmaceutical composition.

Specifically, by including a pharmaceutically acceptable carrier in addition to the active ingredient, the pharmaceutical composition may be prepared into a parenteral formulation depending on its route of administration using conventional methods known in the art. Here, the term “pharmaceutically acceptable” means that the carrier does not have more toxicity than the subject to be applied (prescribed) can adapt while not inhibiting activity of the active ingredient.

When the pharmaceutical composition is prepared into a parenteral formulation, it may be made into preparations in the form of injections, transdermal patches, nasal inhalants, or suppositories with suitable carriers according to methods known in the art. In a case of being made into injections, sterile water, ethanol, polyol such as glycerol or propylene glycol, or a mixture thereof may be used as a suitable carrier; and an isotonic solution, such as Ringer's solution, phosphate buffered saline (PBS) containing triethanol amine or sterile water for injection, and 5% dextrose, or the like may preferably be used. Formulation of pharmaceutical compositions is known in the art, and reference may specifically be made to Remington's Pharmaceutical Sciences (19th ed., 1995) and the like. This document is considered part of the present description.

A preferred dose of the pharmaceutical composition may range from 0.01 μg/kg to 10 g/kg, or 0.01 mg/kg to 1 g/kg, per day, depending on the patient's condition, body weight, sex, age, severity of the patient, and route of administration. The dose may be administered once a day or may be divided into several times a day. Such a dose should not be construed as limiting the scope of the present invention in any aspect.

Subjects to which the pharmaceutical composition can be applied (prescribed) are mammals and humans, with humans being particularly preferred. In addition to the active ingredient, the pharmaceutical composition of the present application may further contain any compound or natural extract, which has already been validated for safety and is known to have anticancer activity or a therapeutic effect on an infectious disease, so as to boost or reinforce anticancer activity.

Method for Enhancing Immunity in a Subject by Using the Fusion Protein Dimer

In still yet another aspect of the present invention, there is provided a method for enhancing immunity in a subject by using the fusion protein dimer. Specifically, the production method may comprise administering the fusion protein dimer according to the aspect of the present invention or the pharmaceutical composition according to the aspect of the present invention to the subject in need thereof.

In an embodiment, the fusion protein dimer may proliferate any lymphocyte. The lymphocyte is a type of white blood cell (leukocyte) in the immune system of most vertebrates. The lymphocyte comprises T cells, natural killer cells, or B cells. Specifically, the lymphocyte is any one selected from the group consisting of a CD8+ cytotoxic T cell, a CD4+ T helper cell, a regulatory T cell, a natural killer cell, and a B cell.

Polynucleotide Encoding Fusion Protein

In yet another aspect of the present invention, there is provided a polynucleotide encoding a fusion protein comprising an IL-2 protein and a CD80 protein. Specifically, the polynucleotide may contain the nucleotide sequence of SEQ ID NO: 8, 25, 27, or 29. The fusion protein comprising an IL-2 protein and a CD80 protein is as described above. In the polynucleotide, one or more nucleotides may be altered by substitution, deletion, insertion, or a combination thereof. When a nucleotide sequence is prepared by chemical synthesis, synthetic methods well known in the art may be used, such as those described in Engels and Uhlmann (Angew Chem IntEd Eng., 37: 73-127, 1988). Such methods may include triester, phosphite, phosphoramidite and H-phosphate methods, PCR and other autoprimer methods, oligonucleotide syntheses on solid supports, and the like. In addition, the fusion protein having a high content of sialic acid is as described above.

According to an embodiment, the polypeptide may contain a nucleic acid sequence having an identity, to SEQ ID NO: 8, 25, 27, or 29, of at least about 70%, at least about 75%, at least about 80%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100%.

The polynucleotide may further contain a nucleic acid encoding a signal sequence or a leader sequence. As used herein, the term “signal sequence” refers to a signal peptide that directs secretion of a target protein. The signal peptide is translated and then cleaved in a host cell. Specifically, the signal sequence is an amino acid sequence that initiates migration of a protein across the endoplasmic reticulum (ER) membrane. In an embodiment, the signal sequence may have the amino acid sequence of SEQ ID NO: 1.

Signal sequences are well known in the art for their characteristics. Such signal sequences typically contain 16 to 30 amino acid residues, and may contain more or fewer amino acid residues than such amino acid residues. A typical signal peptide is composed of three regions, that is, a basic N-terminal region, a central hydrophobic region, and a more polar C-terminal region. The central hydrophobic region contains 4 to 12 hydrophobic residues that cause the signal sequence to be immobilized during migration of an immature polypeptide through the membrane lipid bilayer.

After initiation, signal sequences are cleaved in the lumen of ER by cellular enzymes, commonly known as signal peptidases. Here, the signal sequence may be a secretory signal sequence of tPa (tissue plasminogen activator), HSV gDs (signal sequence of Herpes simplex virus glycoprotein D), or a growth hormone. Preferably, a secretory signal sequence used in higher eukaryotic cells including mammals and the like may be used. In addition, a signal sequence included in the wild-type IL-2 and/or CD-80 may be used, or a signal sequence that has been substituted with a codon having high expression frequency in a host cell may be used.

Vector with Polynucleotide Encoding Fusion Protein

In still yet another aspect of the present invention, there is provided a vector comprising the polynucleotide.

The vector may be introduced into a host cell to be recombined with and inserted into the genome of the host cell. Or, the vector is understood as nucleic acid means containing a polynucleotide sequence which is autonomously replicable as an episome. The vectors include linear nucleic acids, plasmids, phagemids, cosmids, RNA vectors, viral vectors, and analogs thereof. Examples of the viral vector include, but are not limited to, retroviruses, adenoviruses, and adeno-associated viruses.

Specifically, the vector may include plasmid DNA, phage DNA, and the like; and commercially developed plasmids (pUC18, pBAD, pIDTSAMRT-AMP, and the like), E. coli-derived plasmids (pYG601BR322, pBR325, pUC118, pUC119, and the like), Bacillus subtilis-derived plasmids (pUB110, pTP5, and the like), yeast-derived plasmids (YEp13, YEp24, YCp50, and the like), phage DNA (Charon4A, Charon21A, EMBL3, EMBL4, λ gt10, λ gt11, λ ZAP, and the like), animal viral vectors (retroviruses, adenoviruses, vaccinia viruses, and the like), insect viral vectors (baculoviruses and the like). Since the vector exhibits different expression levels and modification of a protein depending on a host cell, it is preferred to select and use a host cell which is most suitable for the purpose.

As used herein, the term “gene expression” or “expression” of a target protein is understood to mean transcription of DNA sequences, translation of mRNA transcripts, and secretion of fusion protein products or fragments thereof. A useful expression vector may be RcCMV (Invitrogen, Carlsbad) or a variant thereof. Expression vectors may further contain human cytomegalovirus (CMV) promoter for promoting continuous transcription of a target gene in mammalian cells, and a bovine growth hormone polyadenylation signal sequence for increasing the stability level of RNA after transcription.

Transformed Cell Expressing Fusion Protein

In still yet another aspect of the present invention, there is provided a transformed cell into which the vector has been introduced.

Host cells for the transformed cell may include, but are not limited to, prokaryotic cells, eukaryotic cells, and cells of mammalian, vegetable, insect, fungal, or bacterial origin. As an example of the prokaryotic cells, E. coli may be used. In addition, as an example of the eukaryotic cells, yeast may be used. In addition, for the mammalian cells, CHO cells, F2N cells, CSO cells, BHK cells, Bowes melanoma cells, HeLa cells, 911 cells, AT1080 cells, A549 cells, HEK 293 cells, HEK293T cells, or the like may be used. However, the mammalian cells are not limited thereto, and any cells which are known to those skilled in the art to be usable as mammalian host cells may be used.

In addition, for the introduction of an expression vector into the host cell, CaCl₂ precipitation, Hanahan method whose efficiency has been increased efficiency by using a reducing agent such as dimethyl sulfoxide (DMSO) in CaCl₂ precipitation, electroporation, calcium phosphate precipitation, protoplast fusion, agitation using silicon carbide fiber, Agrobacteria-mediated transformation, transformation using PEG, dextran sulfate-, Lipofectamine-, or dry/inhibition-mediated transformation, or the like may be used.

As described above, for optimization of properties of a fusion protein as a therapeutic agent or for any other purpose, glycosylation pattern of the fusion protein (for example, sialic acids, fucosylations, glycosylations) may be adjusted by manipulating, through methods known to those skilled in the art, glycosylation-related genes possessed by host cells.

Method for Producing a Fusion Protein

In still yet another aspect of the present invention, there is provided a method for producing a fusion protein comprising an IL-2 protein and a CD80 protein, the method comprising culturing the transformed cells. Specifically, the production method may comprise i) culturing the transformed cells to obtain a culture; and ii) collecting the fusion protein from the culture.

Culturing the transformed cells may be carried out using methods well known in the art. Specifically, the culture may be carried out in a batch process, or carried out continuously in a fed batch or repeated fed batch process.

Use of Fusion Protein Dimer

In still yet another aspect of the present invention, there is provided a use of a fusion protein dimer comprising an IL-2 protein and a CD80 protein and having a high content of sialic acid for treating cancer or an infectious disease.

In still yet another aspect of the present invention, there is provided a use of a fusion protein dimer comprising an IL-2 protein and a CD80 protein and having a high content of sialic acid for enhancing a therapeutic effect on cancer or an infectious disease.

In still yet another aspect of the present invention, there is provided a use of a fusion protein dimer comprising an IL-2 protein and a CD80 protein and having a high content of sialic acid for manufacture of a medicament for treating cancer or an infectious disease.

In still yet another aspect of the present invention, there is provided a method for treating cancer or an infectious disease, and/or a method for enhancing a therapeutic effect on cancer or an infectious disease, comprising administering, to a subject, a fusion protein dimer comprising an IL-2 protein and a CD80 protein and having a high content of sialic acid.

The subject may be an individual suffering from cancer or an infectious disease. In addition, the subject may be a mammal, preferably a human. The fusion protein is as described above.

The cancer may be selected from the group consisting of gastric cancer, liver cancer, lung cancer, colorectal cancer, breast cancer, prostate cancer, ovarian cancer, pancreatic cancer, cervical cancer, thyroid cancer, laryngeal cancer, acute myeloid leukemia, brain tumor, neuroblastoma, retinoblastoma, head and neck cancer, salivary gland cancer, and lymphoma. In addition, the infectious disease may be any one selected from the group consisting of hepatitis B, hepatitis C, human papilloma virus (HPV) infection, cytomegalovirus infection, viral respiratory disease, and influenza.

Route of administration, dose, and frequency of administration of the fusion protein dimer may vary depending on the patient's condition and the presence or absence of side effects, and thus the fusion protein dimer may be administered to a subject in various ways and amounts. The optimal administration method, dose, and frequency of administration can be selected in an appropriate range by those skilled in the art. In addition, the fusion protein dimer may be administered in combination with other drugs or physiologically active substances whose therapeutic effect is known with respect to a disease to be treated, or may be formulated in the form of combination preparations with other drugs.

Due to IL-2 activity, the fusion protein dimer in an embodiment of the present invention can activate immune cells such as natural killer cells. Thus, the fusion protein dimer can be effectively used for cancer and infectious diseases. In particular, it was identified that as compared with the wild type, an IL-2 variant with two to five amino acid substitutions, in particular, an IL-2 variant that comprises amino acid substitutions at two, three, four, or five positions among the positions selected from the group consisting of R38A, F42A, Y45A, E61R, and L72G, has low binding ability for the IL-2 receptor alpha chain and thus exhibits improved characteristics with respect to pharmacological side effects of conventional IL-2. Thus, such an IL-2 variant, when used alone or in the form of a fusion protein, can decrease incidence of vascular (or capillary) leakage syndrome (VLS), a problem with IL-2 conventionally known.

### Mode for the Invention

Hereinafter, the present invention will be described in more detail by way of the following examples. However, the following examples are only for illustrating the present invention, and the scope of the present invention is not limited thereto.

I. Preparation of Fusion Protein

### PREPARATION EXAMPLE 1

### Preparation of hCD80-Fc-IL-2 variant (2M): GI101

In order to produce a fusion protein comprising a human CD80 fragment, an Fc domain, and an IL-2 variant, a polynucleotide was synthesized through the Invitrogen GeneArt Gene Synthesis service of ThermoFisher Scientific. Specifically, the polynucleotide contains a nucleotide sequence (SEQ ID NO: 8) which encodes a fusion protein that contains a signal peptide (SEQ ID NO: 1), a CD80 fragment (SEQ ID NO: 2), an Ig hinge (SEQ ID NO: 3), an Fc domain (SEQ ID NO: 4), a linker (SEQ ID NO: 5), and an IL-2 variant (2M) (R38A, F42A) (SEQ ID NO: 6) having two amino acid substitutions, in this order, from the N-terminus. The polynucleotide was inserted into pcDNA3_4 vector. In addition, the vector was introduced into CHO cells (EXPI-CHO™) to express the fusion protein of SEQ ID NO: 9. After the vector was introduced, culture was performed for 7 days in an environment of 37° C., 125 RPM, and 8% CO₂ concentration. Then, the culture was harvested and the fusion protein was purified therefrom. The purified fusion protein was designated “GI101”.

Purification was carried out using chromatography containing MabSelect SuRe protein A resin. The fusion protein was bound thereto under a condition of 25 mM Tris, 25 mM NaCl, pH 7.4. Then, elution was performed with 100 mM NaCl, 100 mM acetic acid, pH 3. 20% 1 M Tris-HCl at pH 9 was placed in a collection tube, and then the fusion protein was collected. For the collected fusion protein, the buffer was exchanged through dialysis with PBS buffer for 16 hours.

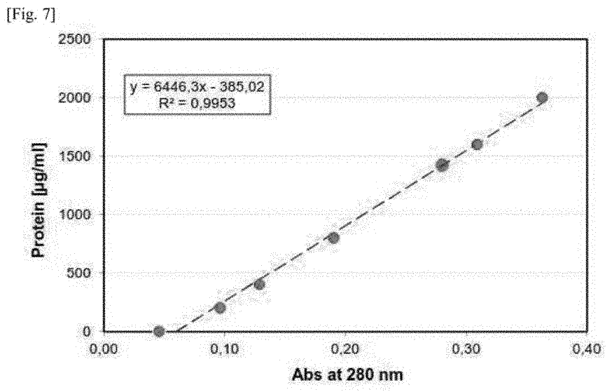

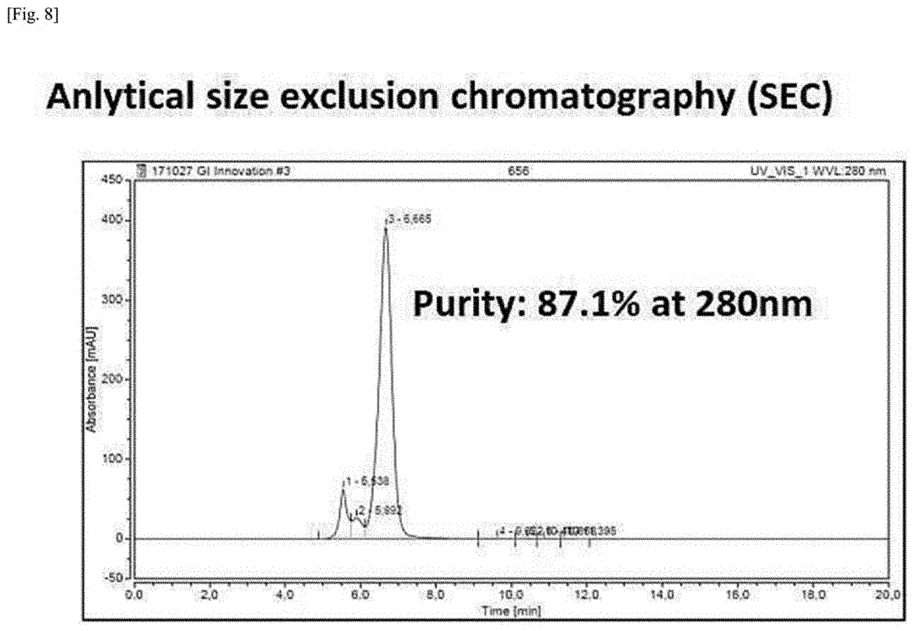

Thereafter, absorbance at 280 nm wavelength was measured, over time, with size exclusion chromatography using a TSKgel G3000SWXL column (TOSOH Bioscience), to obtain a highly concentrated fusion protein. Here, the isolated and purified fusion protein was subjected to SDS-PAGE under reduced (R) or non-reduced (NR) condition, and stained with Coomassie Blue to check its purity (FIG. 6). It was identified that the fusion protein was contained at a concentration of 2.78 mg/ml when detected with NanoDrop (FIG. 7). In addition, the results obtained by analysis using size exclusion chromatography are provided in FIG. 8.

### PREPARATION EXAMPLE 2

### Preparation of mCD80-Fc-IL-2 variant (2M): mGI101

In order to produce a fusion protein comprising a mouse CD80, an Fc domain, and an IL-2 variant, a polynucleotide was synthesized through the Invitrogen GeneArt Gene Synthesis service of ThermoFisher Scientific. Specifically, the polynucleotide contains a nucleotide sequence (SEQ ID NO: 14) which encodes a fusion protein that contains a signal peptide (SEQ ID NO: 1), a mCD80 (SEQ ID NO: 13), an Ig hinge (SEQ ID NO: 3), an Fc domain (SEQ ID NO: 4), a linker (SEQ ID NO: 5), and an IL-2 variant (2M) (R38A, F42A) (SEQ ID NO: 6) with two amino acid substitutions, in this order, from the N-terminus. The polynucleotide was inserted into pcDNA3_4 vector. In addition, the vector was introduced into CHO cells (EXPI-CHO™) to express the fusion protein of SEQ ID NO: 15. After the vector was introduced, culture was performed for 7 days in an environment of 37° C., 125 RPM, and 8% CO₂ concentration. Then, the culture was harvested and the fusion protein was purified therefrom. The purified fusion protein was designated “mGI101”.

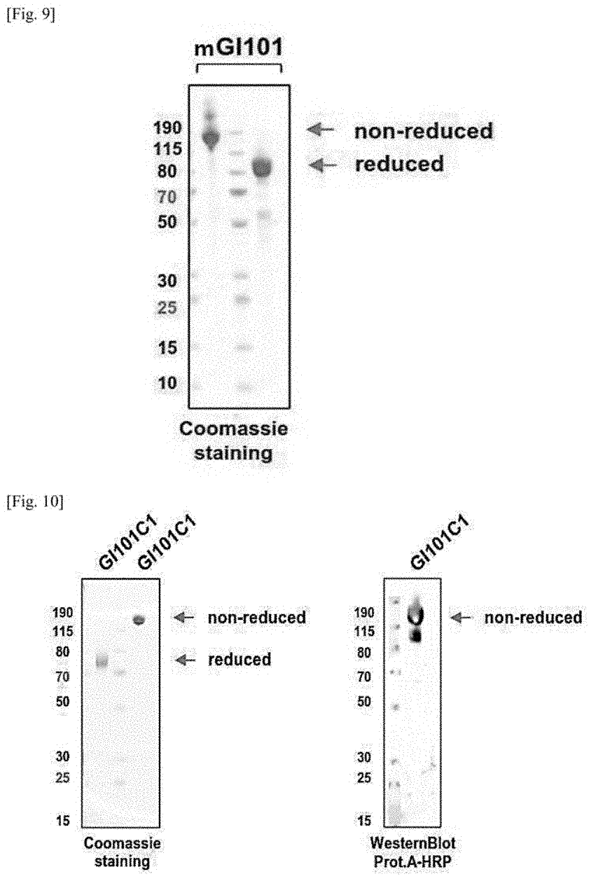

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 9). It was found that the fusion protein was contained at a concentration of 1.95 mg/ml when detected by absorbance at 280 nm using NanoDrop.

### PREPARATION EXAMPLE 3

### Preparation of hCD80-Fc: GI101C1

In order to produce a fusion protein comprising a human CD80 fragment and an Fc domain, a polynucleotide was synthesized through the Invitrogen GeneArt Gene Synthesis service of ThermoFisher Scientific. Specifically, the polynucleotide contains a nucleotide sequence (SEQ ID NO: 16) which encodes a fusion protein that contains a signal peptide (SEQ ID NO: 1), a CD80 fragment (SEQ ID NO: 2), an Ig hinge (SEQ ID NO: 3), and an Fc domain (SEQ ID NO: 4). The polynucleotide was inserted into pcDNA3_4 vector. In addition, the vector was introduced into CHO cells (EXPI-CHO™) to express the fusion protein of SEQ ID NO: 17. After the vector was introduced, culture was performed for 7 days in an environment of 37° C., 125 RPM, and 8% CO₂ concentration. Then, the culture was harvested and the fusion protein was purified therefrom. The purified fusion protein was designated “GI101C1”.

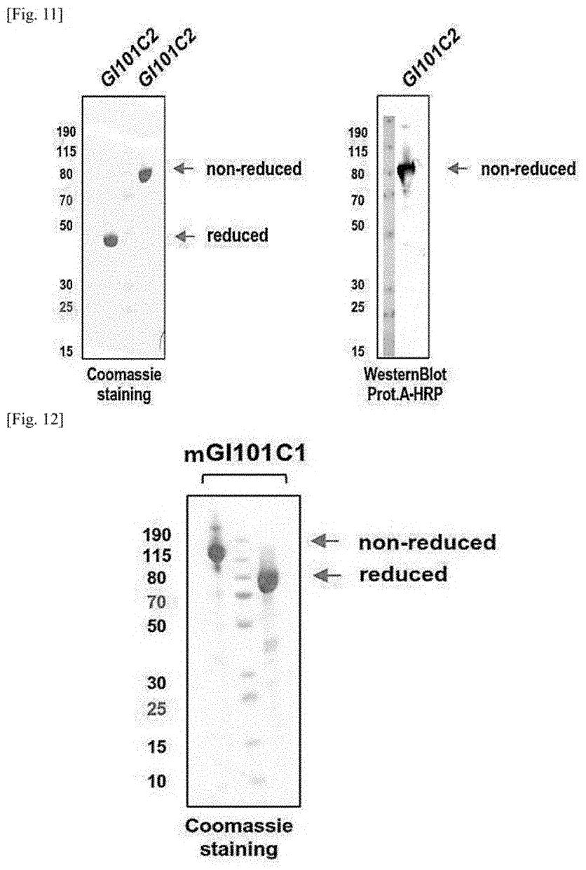

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 10). It was observed that the fusion protein was contained at a concentration of 3.61 mg/ml when detected by absorbance at 280 nm using NanoDrop.

### PREPARATION EXAMPLE 4

### Preparation of Fc-IL-2 variant (2M): GI101C2

In order to produce a fusion protein comprising an Fc domain and an IL-2 variant, a polynucleotide was synthesized through the Invitrogen GeneArt Gene Synthesis service of ThermoFisher Scientific. Specifically, the polynucleotide contains a nucleotide sequence (SEQ ID NO: 18) which encodes a fusion protein that contains a signal peptide (SEQ ID NO: 1), an Fc domain (SEQ ID NO: 4), a linker (SEQ ID NO: 5), and an IL-2 variant (2M) (R38A, F42A) (SEQ ID NO: 6) with two amino acid substitutions, in this order, from the N-terminus. The polynucleotide was inserted into pcDNA3_4 vector. In addition, the vector was introduced into CHO cells (EXPI-CHO™) to express the fusion protein of SEQ ID NO: 19. After the vector was introduced, culture was performed for 7 days in an environment of 37° C., 125 RPM, and 8% CO₂ concentration. Then, the culture was harvested and the fusion protein was purified therefrom. The purified fusion protein was designated “GI101C2”.

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 11). It was found that the fusion protein was contained at a concentration of 4.79 mg/ml when detected by absorbance at 280 nm using NanoDrop.

### PREPARATION EXAMPLE 5

### Preparation of mCD80-Fc: mGI101C1

In order to produce a fusion protein comprising a mouse CD80 and an Fc domain, a polynucleotide was synthesized through the Invitrogen GeneArt Gene Synthesis service of ThermoFisher Scientific. Specifically, the polynucleotide contains a nucleotide sequence (SEQ ID NO: 20) which encodes a fusion protein that contains a signal peptide (SEQ ID NO: 1), a mCD80 (SEQ ID NO: 13), an Ig hinge (SEQ ID NO: 3), and an Fc domain (SEQ ID NO: 4), in this order, from the N-terminus. The polynucleotide was inserted into pcDNA3_4 vector. In addition, the vector was introduced into CHO cells (EXPI-CHO™) to express the fusion protein of SEQ ID NO: 21. After the vector was introduced, culture was performed for 7 days in an environment of 37° C., 125 RPM, and 8% CO₂ concentration. Then, the culture was harvested and the fusion protein was purified therefrom. The purified fusion protein was designated “mGI101C1”.

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 12). It was observed that the fusion protein was contained at a concentration of 2.49 mg/ml when detected by absorbance at 280 nm using NanoDrop.

The fusion proteins prepared in Preparation Examples 1 to 5 are summarized in Table 1 below.

TABLE 1

| Item | N-terminus | Linker | C-terminus |
|---|---|---|---|
| Preparation Example | hCD80 | Fc domain | hIL-2m |
| 1 (GI101) | fragment | | |
| Preparation Example | mCD80 | Fc domain | hIL-2m |
| 2 (mGI101) | fragment | | |
| Preparation Example | CD80 | Fc domain | — |
| 3 (GI101C1) | fragment | | |
| Preparation Example | — | Fc domain | IL-2m |
| 4 (GI101C2) | | | |
| Preparation Example | mCD80 | Fc domain | — |
| 5 (mGI101C1) | fragment | | |

### PREPARATION EXAMPLE 6

### Preparation of CD80-Fc-IL-2: GI101w

In order to produce a fusion protein comprising a human CD80 fragment, an Fc domain, and a human IL-2, a polynucleotide was synthesized through the Invitrogen GeneArt Gene Synthesis service of ThermoFisher Scientific. Specifically, the polynucleotide contains a nucleotide sequence (SEQ ID NO: 31) which encodes a fusion protein that contains a signal peptide (SEQ ID NO: 1), a CD80 fragment (SEQ ID NO: 2), an Ig hinge (SEQ ID NO: 3), an Fc domain (SEQ ID NO: 4), a linker (SEQ ID NO: 5), and mature human IL-2 (SEQ ID NO: 10), in this order, from the N-terminus. The polynucleotide was inserted into pcDNA3_4 vector. In addition, the vector was introduced into CHO cells (EXPI-CHO™) to express the fusion protein of SEQ ID NO: 32. After the vector was introduced, culture was performed for 7 days in an environment of 37° C., 125 RPM, and 8% CO₂ concentration. Then, the culture was harvested and the fusion protein was purified therefrom. The purified fusion protein was designated “GI101w”. The purification and collection of the fusion protein were carried out in the same manner as in Preparation Example 1.

### PREPARATION EXAMPLE 7

### Preparation of hCD80-Fc-IL-2 variant (3M): GI102-M45

In order to produce a fusion protein comprising a human CD80 fragment, an Fc domain, and an IL-2 variant (3M) (R38A, F42A, Y45A) (GI102-M45) with three amino acid substitutions, a polynucleotide was synthesized through the Invitrogen GeneArt Gene Synthesis service of ThermoFisher Scientific. Specifically, the polynucleotide contains a nucleotide sequence (SEQ ID NO: 25) which encodes a fusion protein that contains a signal peptide (SEQ ID NO: 1), a CD80 fragment (SEQ ID NO: 2), an Ig hinge (SEQ ID NO: 3), an Fc domain (SEQ ID NO: 4), a linker (SEQ ID NO: 5), and an IL-2 variant (SEQ ID NO: 22), in this order, from the N-terminus. The polynucleotide was inserted into pcDNA3_4 vector. In addition, the vector was introduced into CHO cells (EXPI-CHO™) to express the fusion protein of SEQ ID NO: 26. After the vector was introduced, culture was performed for 7 days in an environment of 37° C., 125 RPM, and 8% CO₂ concentration. Then, the culture was harvested and the fusion protein was purified therefrom. The purified fusion protein was designated “GI102-M45”.

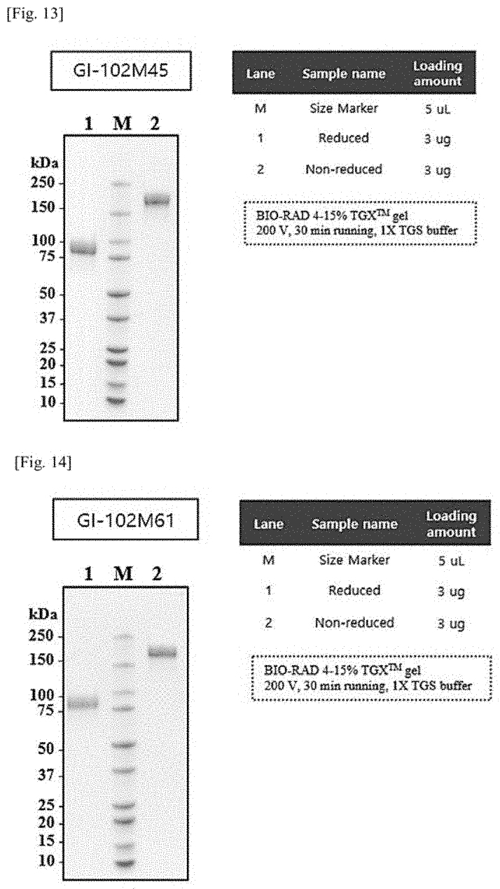

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 13).

### PREPARATION EXAMPLE 8

### Preparation of hCD80-Fc-IL-2 variant (3M): G1102-M61

In order to produce a fusion protein comprising a human CD80 fragment, an Fc domain, and an IL-2 variant (3M) (R38A, F42A, E61R) (GI102-M61) with three amino acid substitutions, a polynucleotide was synthesized through the Invitrogen GeneArt Gene Synthesis service of ThermoFisher Scientific. Specifically, the polynucleotide contains a nucleotide sequence (SEQ ID NO: 27) which encodes a fusion protein that contains a signal peptide (SEQ ID NO: 1), a CD80 fragment (SEQ ID NO: 2), an Ig hinge (SEQ ID NO: 3), an Fc domain (SEQ ID NO: 4), a linker (SEQ ID NO: 5), and an IL-2 variant (SEQ ID NO: 23), in this order, from the N-terminus. The polynucleotide was inserted into pcDNA3_4 vector. In addition, the vector was introduced into CHO cells (EXPI-CHO™) to express the fusion protein of SEQ ID NO: 28. After the vector was introduced, culture was performed for 7 days in an environment of 37° C., 125 RPM, and 8% CO₂ concentration. Then, the culture was harvested and the fusion protein was purified therefrom. The purified fusion protein was designated “GI102-M61”.

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 14).

### PREPARATION EXAMPLE 9

### Preparation of hCD80-Fc-IL-3M: GI102-M72

In order to produce a fusion protein comprising a human CD80 fragment, an Fc domain, and an IL-2 variant (3M) (R38A, F42A, L72G) (GI102-M72) with three amino acid substitutions, a polynucleotide was synthesized through the Invitrogen GeneArt Gene Synthesis service of ThermoFisher Scientific. Specifically, the polynucleotide contains a nucleotide sequence (SEQ ID NO: 29) which encodes a fusion protein that contains a signal peptide (SEQ ID NO: 1), a CD80 fragment (SEQ ID NO: 2), an Ig hinge (SEQ ID NO: 3), an Fc domain (SEQ ID NO: 4), a linker (SEQ ID NO: 5), and an IL-2 variant (SEQ ID NO: 24), in this order, from the N-terminus. The polynucleotide was inserted into pcDNA3_4 vector. In addition, the vector was introduced into CHO cells (EXPI-CHO™) to express the fusion protein of SEQ ID NO: 30. After the vector was introduced, culture was performed for 7 days in an environment of 37° C., 125 RPM, and 8% CO₂ concentration. Then, the culture was harvested and the fusion protein was purified therefrom. The purified fusion protein was designated “GI102-M72”.

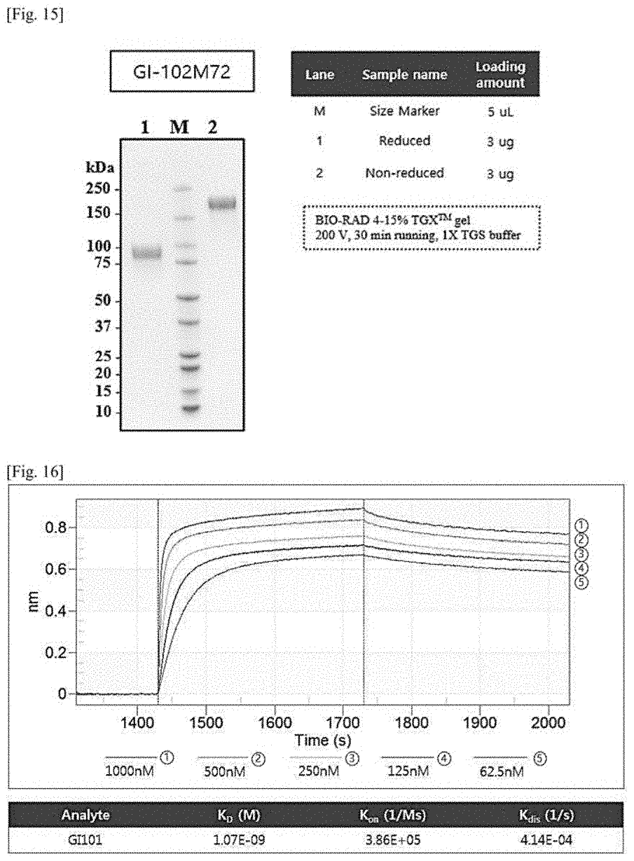

The purification and collection of the fusion protein were carried out in the same manner as in Preparation Example 1. The isolated and purified fusion protein was subjected to SDS-PAGE under reduced (R) or non-reduced (NR) condition and stained with Coomassie Blue to check its purity (FIG. 15).

### PREPARATION EXAMPLE 10

### Preparation of mCD80-Fc-IL-3M: mGI102-M61

In order to produce a fusion protein comprising a mouse CD80 fragment, an Fc domain, and an IL-2 variant (3M) (R38A, F42A, E61R) (GI102-M61) with three amino acid substitutions, a polynucleotide was synthesized through the Invitrogen GeneArt Gene Synthesis service of ThermoFisher Scientific. Specifically, the polynucleotide contains a nucleotide sequence (SEQ ID NO: 33) which encodes a fusion protein that contains a signal peptide (SEQ ID NO: 1), a mCD80 fragment (SEQ ID NO: 13), an Ig hinge (SEQ ID NO: 3), an Fc domain (SEQ ID NO: 4), a linker (SEQ ID NO: 5), and an IL-2 variant (SEQ ID NO: 23), in this order, from the N-terminus. The polynucleotide was inserted into pcDNA3_4 vector. In addition, the vector was introduced into CHO cells (EXPI-CHO™) to express the fusion protein of SEQ ID NO: 34. After the vector was introduced, culture was performed for 7 days in an environment of 37° C., 125 RPM, and 8% CO₂ concentration. Then, the culture was harvested and the fusion protein was purified therefrom. The purified fusion protein was designated “mGI102-M61”.

The purification and collection of the fusion protein were carried out in the same manner as in Preparation Example 1.

II. Identification of Binding Affinity Between Fusion Protein and its Ligand

In order to identify the binding affinity between the fusion protein and its ligand, the binding affinity was measured using Octet RED 384.

### EXPERIMENTAL EXAMPLE 1

### Identification of Binding Affinity Between hCTLA-4 and GI101

AR2G biosensor (Amine Reactive 2^{nd} gen, ForteBio, Cat: 18-5092) was previously hydrated with 200 μl of distilled water in a 96-well microplate (GreinerBio-one, Cat: 655209). A ligand (CTLA-4, Human CTLA-4/CD152, His tag, Sino Biological, Cat: 11159-H08H) to be attached to the AR2G biosensor was diluted with 10 mM acetate buffer (pH 5, AR2G reagent Kit, ForteBio, Cat: 18-5095) to a concentration of 5 μg/ml. In addition, GI101 to be attached to the ligand was diluted with 1× AR2G kinetic buffer (AR2G reagent Kit, ForteBio, Cat: 18-5095) to a concentration of 1,000 nM, 500 nM, 250 nM, 125 nM, or 62.5 nM. Activation buffer was prepared by mixing 20 mM EDC and 10 mM s-NHS (AR2G reagent Kit, ForteBio, Cat: 18-5095) in distilled water. 80 μl of each reagent was placed in a 384-well microplate (Greiner Bio-one, Cat: 781209) and the program was set up.

As a result, the binding affinity between hCTLA-4 and GI101 was measured as illustrated in FIG. 16.

### EXPERIMENTAL EXAMPLE 2

### Identification of Binding Affinity Between hPD-L1/GI101 and hPD-L1/PD-1

Ni-NTA (Nickel charged Tris-NTA, Ni-NTA Biosensors, ForteBio, 18-5101) was previously hydrated with 200 μl of 1× Ni-NTA kinetic buffer (10× Kinetics buffer, ForteBio, 18-1042) in a 96-well microplate (GreinerBio-one, Cat: 655209). A ligand (Human PD-L1/B7-H1 protein, His-tag, Sino biological, Cat: 10084-H08H) to be attached to the Ni-NTA Biosensors was diluted with 1× Ni-NTA kinetic buffer to a concentration of 5 μg/ml. GI101 to be attached to the ligand was diluted with 1× Ni-NTA kinetic buffer at 1,000 nM, 500 nM, 250 nM, 125 nM, or 62.5 nM. In addition, human PD-1/PDCD1 (Human PD-1/PDCD1, Fc Tag, Sino Biological, Cat: 10377-HO2H) to be attached to the ligand was diluted with 1× Ni-NTA kinetic buffer to a concentration of 2,000 nM, 1,000 nM, 500 nM, 250 nM, or 125 nM. Then, 80 μl of each reagent was placed in a 384-well microplate and the program was set up.

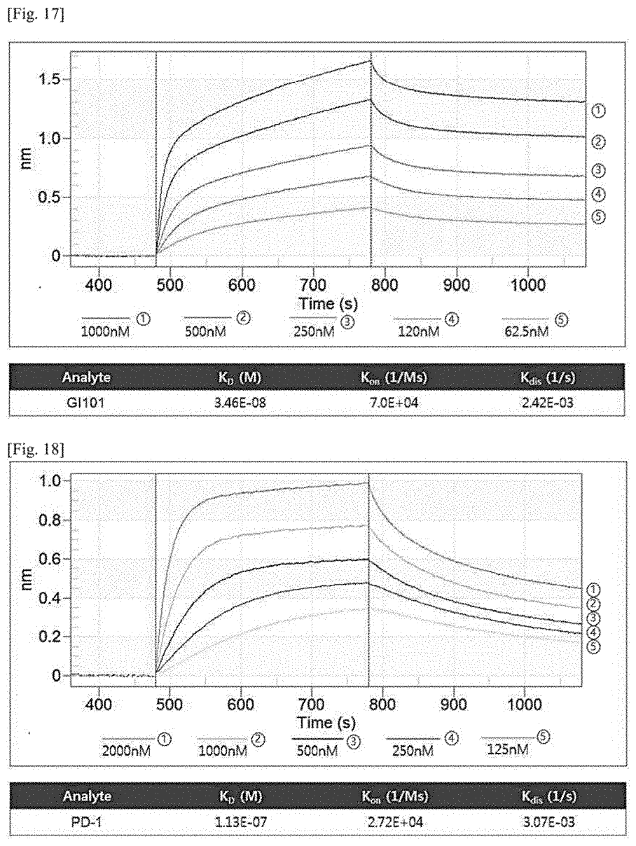

As a result, the binding affinity between hPD-L1 and GI101 was measured as illustrated in FIG. 17. In addition, the binding affinity between hPD-L1 and hPD-1 was measured as illustrated in FIG. 18.

### EXPERIMENTAL EXAMPLE 3

### Identification of Binding Affinity Between mCTLA-4 and mGI101

The binding affinity between mCTLA-4 and mGI101 was examined in the same manner as in Experimental Example 1. Here, the equipment used is as follows: Biosensor: AR2G, Ligand: mCTLA-4 (Recombinant Mouse CTLA-4 Fc chimera, R&D Systems, Cat: 434-CT-200), Analyte: mGI101 (500 nM, 250 nM, 125 nM, 62.5 nM, 31.3 nM).

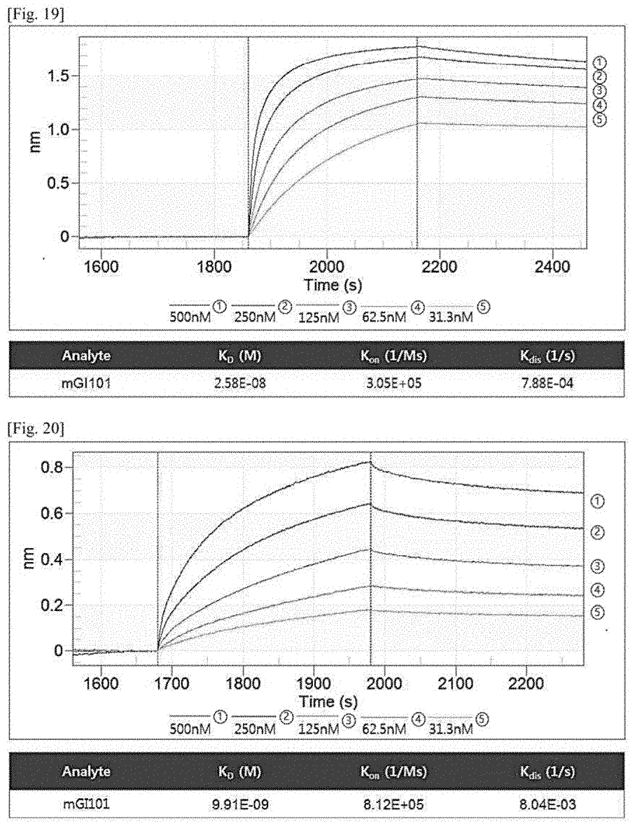

As a result, the binding affinity between mCTLA-4 and mGI101 was measured as illustrated in FIG. 19.

### EXPERIMENTAL EXAMPLE 4

### Identification of Binding Affinity Between mPD-L1 and mGI101

The binding affinity between mPD-L1 and mGI101 was identified in the same manner as in Experimental Example 1. Here, the equipment used is as follows. Biosensor: AR2G, Ligand: mPD-L1 (Recombinant Mouse B7-H1/PD-L1 Fc chimera, R&D Systems, Cat: 434-CT-200), Analyte: mGI101 (500 nM, 250 nM, 125 nM, 62.5 nM, 31.3 nM).

As a result, the binding affinity between mPD-L1 and mGI101 was measured as illustrated in FIG. 20.

### EXPERIMENTAL EXAMPLE 5

### Identification of Binding Ability of GI-101 (hCD80-Fc-hIL-2v) to CTLA-4 and PD-L1

Binding kinetics measurements were performed using the Octet RED 384 instrument (ForteBio, Pall Life Science) with agitation at 30° C. and 1,000 rpm. The binding ability for CTLA-4 was measured using the Amine Reactive 2 generation (AR2G) biosensor chip, and the binding ability for PD-L1 was measured using the Nickel charged Tris-NTA (Ni-NTA) biosensor chip. The AR2G biosensor chip was activated with a combination of 400 mM EDC and 100 mM sulfo-NHS. Then, Human CTLA-4-His Tag (Sino Biological, Cat: 11159-H08H) was diluted with 10 mM acetate buffer (pH 5) to 5 μg/ml, and loaded on the AR2G biosensor chip for 300 seconds and fixed.

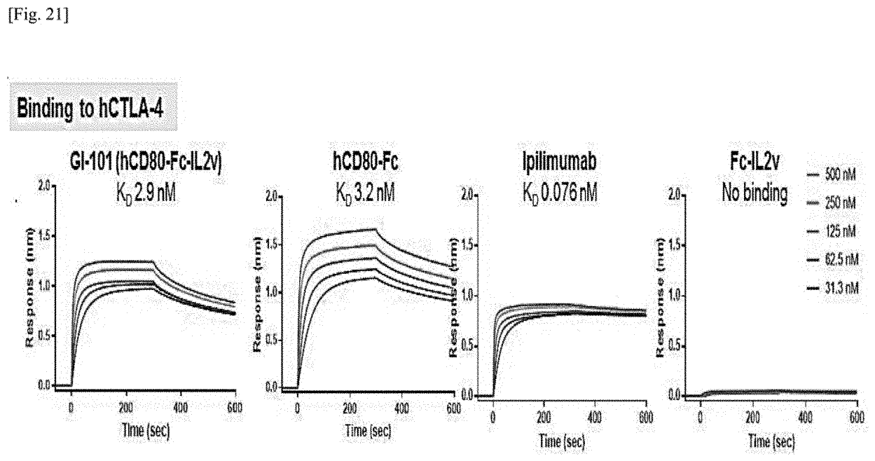

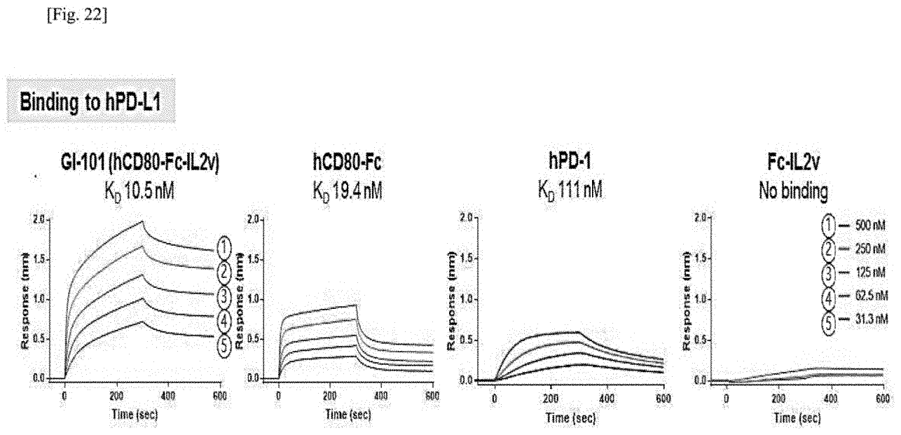

Then, binding of CTLA-4 to GI-101 (hCD80-Fc-hIL-2v), GI-101C1 (hCD80-Fc), Ipilimumab (Bristol-Myers Squibb), and GI-101C2 (Fc-hIL-2v) at various concentrations was measured for 300 seconds and dissociation thereof was also measured for 300 seconds. On the other hand, Human PD-L1-His Tag (Sino biological, Cat: 10084-H08H) was diluted with 1XNi-NTA kinetic buffer to a concentration of 5 μg/ml, and loaded on the Ni-NTA biosensor chip for 600 seconds and fixed. Then, binding of PD-L1 to GI-101, GI-101C1, hPD-1-Fc (Sino biological, Cat: 10377-H02H), and GI101C2 at various concentrations was measured for 300 seconds and dissociation thereof was also measured for 300 seconds. Binding kinetics analysis was performed using Octet Data Analysis HT software ver. 10 provided by Pall Corporation. The results are illustrated in FIGS. 21 and 22.

### EXPERIMENTAL EXAMPLE 6

### Identification of Effect of GI-101 (hCD80-Fc-hIL-2v) on PD-1/PD-L1 Binding

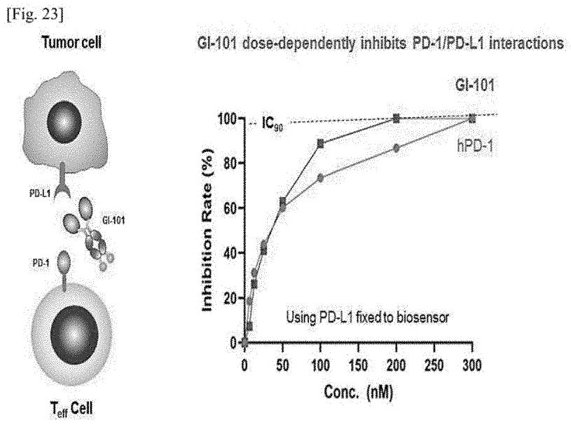

A blocking experiment was performed using the Octet RED 384 instrument (ForteBio, Pall Life Science) with agitation at 30° C. and 1,000 rpm. Human PD-L1-His Tag (Sino biological, Cat: 10084-H08H) was diluted with 1× Ni-NTA kinetic buffer to a concentration of 5 μg/ml, and loaded on the Ni-NTA biosensor chip for 600 seconds and fixed. In order to proceed with the blocking experiment, hPD-L1 fixed on the biosensor chip was allowed to bind to GI-101 at various concentrations (300 nM, 100 nM, 50 nM, 25 nM, 12.5 nM, and 0 nM) for 600 seconds, and then again allowed to bind to the competitor human PD-1 (100 nM) for 600 seconds so as to measure how much more hPD-1 can bind thereto. On the contrary, hPD-L1 was allowed to bind to hPD-1 at various concentrations (300 nM, 100 nM, 50 nM, 25 nM, 12.5 nM, and 0 nM) for 600 seconds, and then again allowed to bind to the competitor GI-101 (100 nM) for 600 seconds so as to measure how much more GI-101 can bind thereto. The blocking experiment was analyzed using the epitope binning menu of Octet Data Analysis HT software ver. 10 provided by Pall Corporation. The results are illustrated in FIG. 23.

### EXPERIMENTAL EXAMPLE 7

### Identification of Binding Affinity Between IL-2Rα or IL-2Rβ and GI101

The binding ability for IL-2Rα was measured using the AR2G biosensor, and the binding ability for IL-2Rβ was measured using the Ni-NTA biosensors (Nickel charged Tris-NTA, Ni-NTA Biosensors, ForteBio, 18-5101).

A ligand (IL-2Rα-His Tag, Acro, Cat: ILA-H52H9) to be attached to the AR2G biosensor was diluted with 10 mM acetate buffer (pH 5, AR2G reagent Kit, ForteBio, Cat: 18-5095) to a concentration of 5 μg/ml. The AR2G biosensor was activated with a buffer prepared by mixing 400 mM EDC and 100 mM sulfo-NHS, and then the diluted ligand was loaded on the AR2G biosensor for 300 seconds and fixed.

Meanwhile, a ligand (IL-2R13-His Tag, Acro, Cat: CD2-H5221) to be attached to the Ni-NTA biosensor was diluted with 1× Ni-NTA kinetic buffer to a concentration of 5 μg/ml. The diluted ligand was loaded on the Ni-NTA biosensor for 600 seconds and fixed.

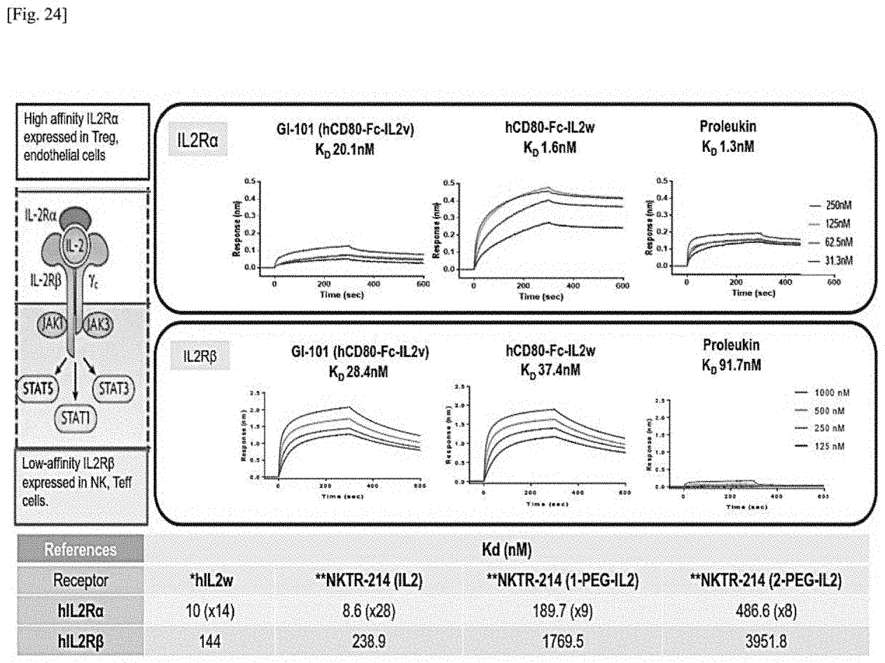

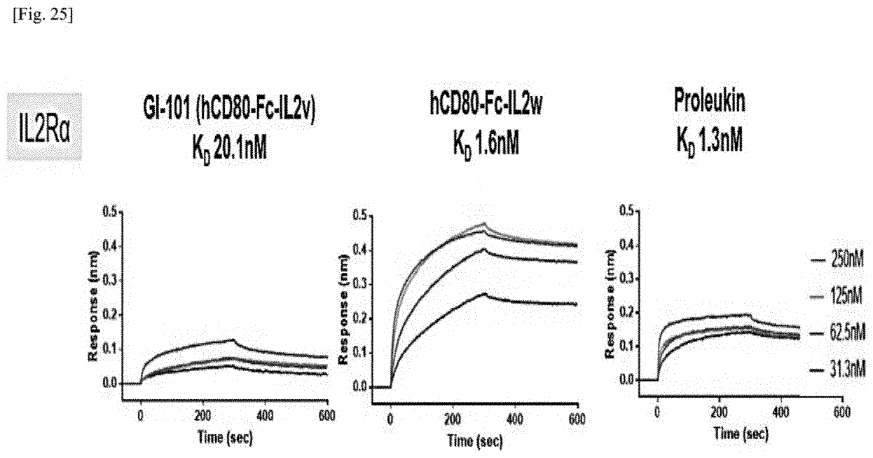

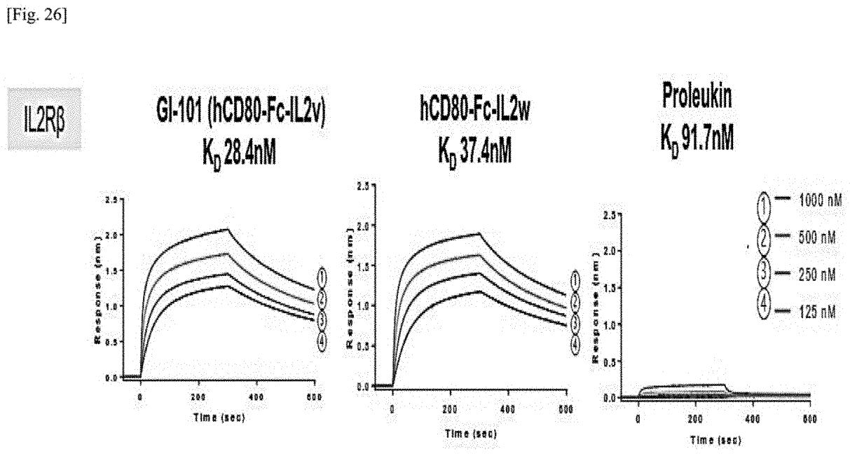

Thereafter, GI101, GI101w, or Proleukin (Novartis, hIL-2), at various concentrations, to be attached to the ligand was loaded thereon for 300 seconds. Then, binding thereof was measured and dissociation thereof was also measured for 300 seconds. Binding kinetics analysis was performed using Octet Data Analysis HT software ver. 10 provided by Pall Corporation. The results are illustrated in FIGS. 24 to 26.

As a result, it was identified that GI101 has low binding ability for the IL-2 receptor alpha chain, IL-2Rα, and high binding ability for IL-2R13, as compared with GI101w and Proleukin.

### EXPERIMENTAL EXAMPLE 8

### Measurement of Binding Affinity Between Fusion Protein and Ligand

In order to identify binding affinity between the fusion protein and its ligand, binding affinity was measured using Octet RED 384.

### EXPERIMENTAL EXAMPLE 8.1

### Identification of Binding Affinity Between IL2 Alpha Receptor and GI101-M45, GI101-M61, or GI101-M72

AR2G biosensor (Amine Reactive 2nd gen, ForteBio, Cat: 18-5092) was previously hydrated with 200 μl of distilled water (DW) in a 96-well microplate (GreinerBio-one, Cat: 655209). A ligand (Human IL-2 R alpha protein, His Tag, Acro, ILA-H52H9) to be attached to the biosensor was diluted with 10 mM acetate buffer (pH 5) (AR2G reagent Kit, ForteBio, Cat: 18-5095) to a concentration of 5 μg/ml. An analyte (GI101-M45, GI101-M61, GI101-M72) to be attached to the ligand was diluted with 1× AR2G kinetic buffer (AR2G reagent Kit, ForteBio, Cat: 18-5095) to 500 nM, 250 nM, 125 nM, and 62.5 nM, respectively. Activation buffer was prepared by mixing 20 mM EDC and 10 mM s-NHS (AR2G reagent Kit, ForteBio, Cat: 18-5095) in DW. 80 μl of each reagent was placed in a 384-well microplate (Greiner Bio-one, Cat: 781209) and the program was set up.

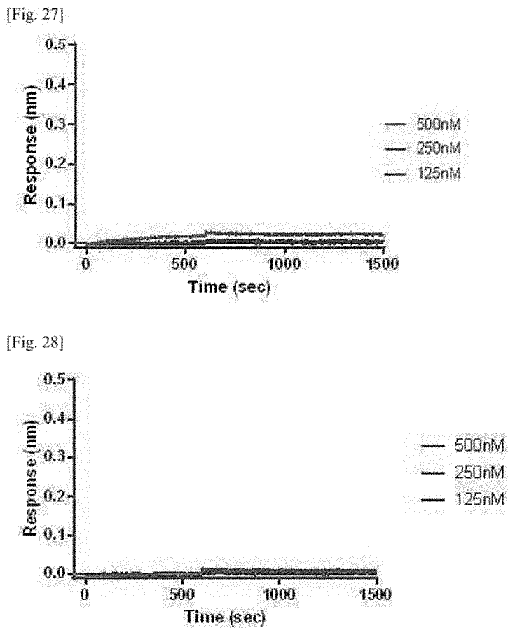

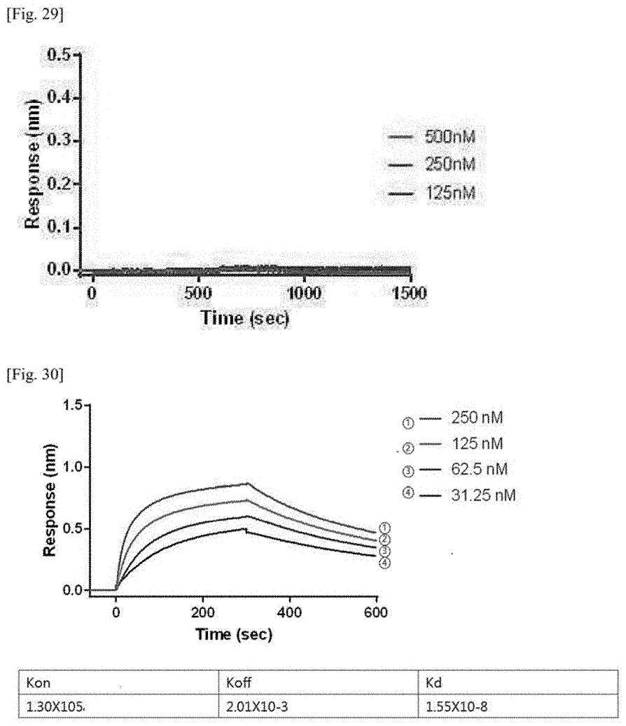

As a result, the binding affinity between IL2 alpha receptor and GI101-M45 is illustrated in FIG. 27. In addition, the binding affinity between IL2 alpha receptor and GI101-M61 is illustrated in FIG. 28, and the binding affinity between IL2 alpha receptor and GI101-M72 is illustrated in FIG. 29.

### EXPERIMENTAL EXAMPLE 8.2

### Identification of Binding Affinity of GI102-M45, GI102-M61, and GI102-M72 to IL-2Rβ

Ni-NTA Biosensors were previously hydrated with 200 μl of 1× Ni-NTA kinetic buffer (10× Kinetics buffer, ForteBio, 18-1042) in a 96-well microplate. A ligand (Human IL-2 R beta protein, His-Tag, Acro, CD2-H5221) to be attached to the biosensor was diluted with 1× Ni-NTA kinetic buffer to a concentration of 2 μg/ml. GI102-M45, GI102-M61, or GI102-M72 to be attached to the ligand was diluted with 1× Ni-NTA kinetic buffer to a concentration of 500 nM, 250 nM, 125 nM, or 62.5 nM. 80 μl of each reagent was placed in a 384-well microplate and the program was set up.

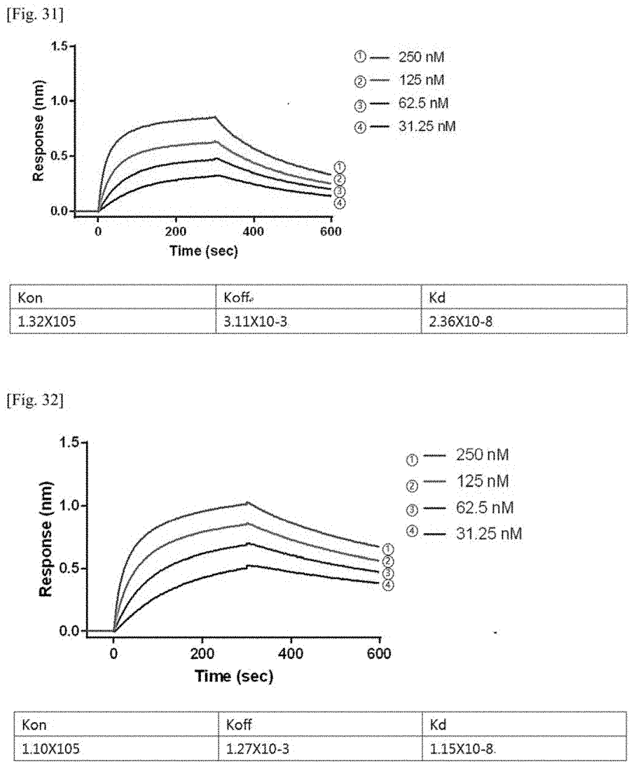

As a result, the binding affinity between IL-2Rβ and GI102-M45 was measured as illustrated in FIG. 30, and the binding affinity between IL-2Rβ and GI102-M61 was measured as illustrated in FIG. 31. In addition, the binding affinity between IL-2R13 and GI102-M72 was measured as illustrated in FIG. 32.

III. Identification of Immune Activity of Fusion Protein

### EXPERIMENTAL EXAMPLE 9

### Identification of IFN-γ Production Caused by Fusion Protein

### EXPERIMENTAL EXAMPLE 9.1

### Culture of CFSE-Labeled PBMCs

Peripheral blood mononuclear cells (PBMCs) isolated from a human were labeled with carboxyfluorescein succinimidyl ester (CF SE) by being reacted with 1 μM CellTrace CFSE dye at 37° C. for 20 minutes. CFSE not bound to the cells was removed by being reacted for 5 minutes with a culture medium having a 5-fold volume of the staining reaction solution and then by being centrifuged at 1,300 rpm for 5 minutes. The CFB-labeled PBMCs were resuspended in the culture medium (RPMI1640 medium containing 10% FBS, 10 mM HEPES, 100 U/ml penicillin/streptomycin, 1 mM sodium pyruvate, 55 μM 2-mercaptoethanol, 1 mM non-essential amino acid, and 2 mM L-glutamine), and then added to a 96-well plate at 1×10⁵ cells per well. Treatment with 5 μg/ml of PHA (Lactin from Phaseolus Vulgaris, red kidney bean, Sigma-Aldrich, St. Louis, Mo., USA, Cat. No. L1668-5MG), and GI101, GI101C1, GI101C2, or IL-2 (Aldesleukin; human recombinant IL-2, Novartis) was performed and incubation was performed in a 5% CO₂ incubator at 37° C. for 6 days.

Here, the treatment with GI101, GI101C1, GI101C2, and IL-2 was performed at a concentration of 1 nM, 10 nM, or 100 nM. The cells were analyzed by FACS, and human IFN-γ present in the culture medium was measured using an ELISA kit (Biolegend, San Diego, Calif., USA, Cat. No. 430103).

### EXPERIMENTAL EXAMPLE 9.2

### FACS Analysis

The cell pellets obtained by removing the supernatant were washed with FACS buffer (3% FBS, 10 mM EDTA, 1M HEPES, 100 unit/mL Penicillin Streptomycin, 10 μg/ml, 1 mM sodium pyruvate), and then reacted with Fc blocker (Biolegend, Cat. No. 422302) at 4° C. for 5 minutes. Then, treatment with APC anti-CD3 Ab (Biolegend, Cat. No. 300412) and PE anti-CD8a Ab (Biolegend, Cat. No. 300908) was performed and reaction was allowed to proceed at 4° C. for 20 minutes. Then, the resultant was washed with FACS buffer. The cell pellets were resuspended in FACS buffer and then analyzed using BD LSR Fortessa (BD Biosciences, San Diego, Calif., USA) and FlowJo software.

### EXPERIMENTAL EXAMPLE 9.3

### Human IFN-γ ELISA

The amount of human IFN-γ secreted into the supernatant of each sample in which the cells had been cultured was measured using a human IFN-γ ELISA kit (Biolegend, Cat. No. 430103). Briefly, anti-human-IFN-γ antibodies were added to an ELISA plate, and reaction was allowed to proceed overnight at 4° C. so that these antibodies were coated thereon. Then, blocking was performed at room temperature for 1 hour with a PBS solution to which 1% BSA had been added. Washing with a washing buffer (0.05% Tween-20 in PBS) was performed, and then a standard solution and each sample were properly diluted and added thereto. Then, reaction was allowed to proceed at room temperature for 2 hours.

After the reaction was completed, the plate was washed and secondary antibodies (detection antibodies) were added thereto. Reaction was allowed to proceed at room temperature for 1 hour. Washing with a washing buffer was performed, and then an Avidin-HRP solution was added thereto. Reaction was allowed to proceed at room temperature for 30 minutes. A substrate solution was added thereto and color development reaction was induced in the dark at room temperature for 20 minutes. Finally, H₂SO₄ was added thereto to stop the color development reaction, and the absorbance at 450 nm was measured with Epoch Microplate Spectrophotometer (BioTek Instruments, Inc., Winooski, Vt., USA).

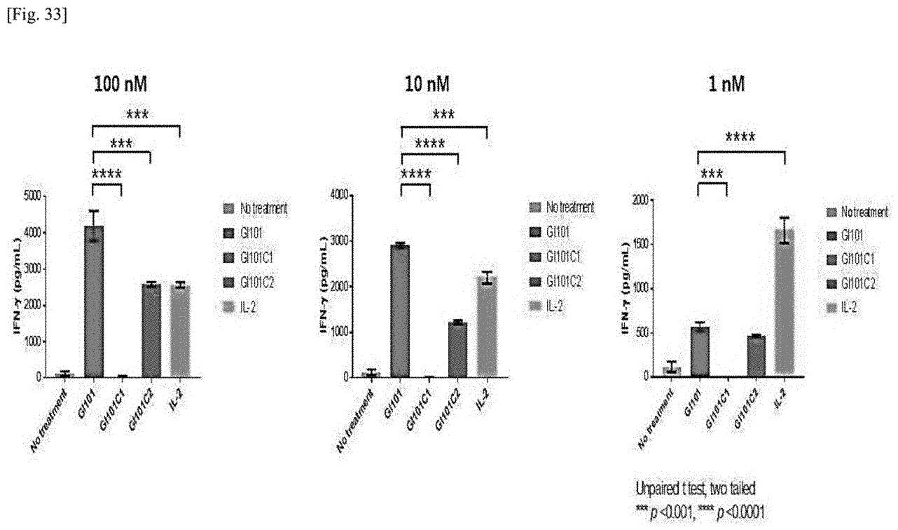

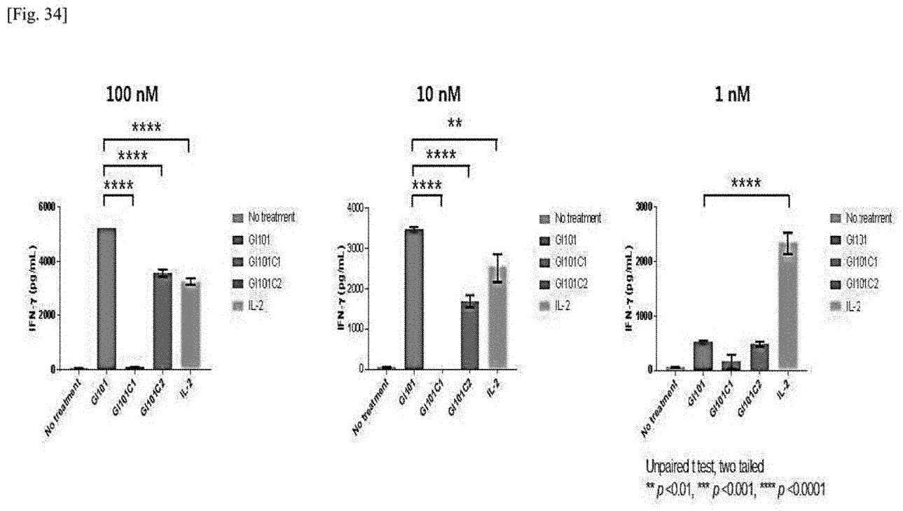

As a result, it was found that cells treated with GI101 exhibited a remarkable increase in IFN-γ secretion, as compared with cells treated with GI101C1, GI101C2, or IL-2 (FIGS. 33 and 34).

### EXPERIMENTAL EXAMPLE 10

### Identification of Effect of GI101 on Proliferation of CD8+ T Cells

Peripheral blood mononuclear cells (PBMCs) isolated from a human were labeled with CFSE by being reacted with 1 μM CellTrace CFSE dye at 37° C. for 20 minutes. CFSE not bound to the cells was removed by being reacted for 5 minutes with a culture medium having a 5-fold volume of the staining reaction solution and then by being centrifuged at 1,300 rpm for 5 minutes. The CFB-labeled PBMCs were resuspended in the culture medium (RPMI1640 medium containing 10% FBS, 10 mM HEPES, 100 U/ml penicillin/streptomycin, 1 mM sodium pyruvate, 55 μM 2-mercaptoethanol, 1 mM non-essential amino acid, and 2 mM L-glutamine), and then added to a 96-well plate at 1×10⁵ cells per well.

Thereafter, treatment with 1 μg/ml of anti-CD3ε antibody (Biolegend Cat. No. L1668-5MG), and GI101, GI101C1, GI101C2, or Proleukin (Novartis) was performed and incubation was performed in a 5% CO₂ incubator at 37° C. for 6 days. Here, the cells were treated with GI101, GI101C1, GI101C2, and IL-2 at a concentration of 100 nM. The incubated cells were examined for their degree of proliferation by measuring, with FACS analysis using APC-TCRαβ and PE-CD8α antibodies, a proportion of CD8+ T cells that had not been labeled with CFSE.

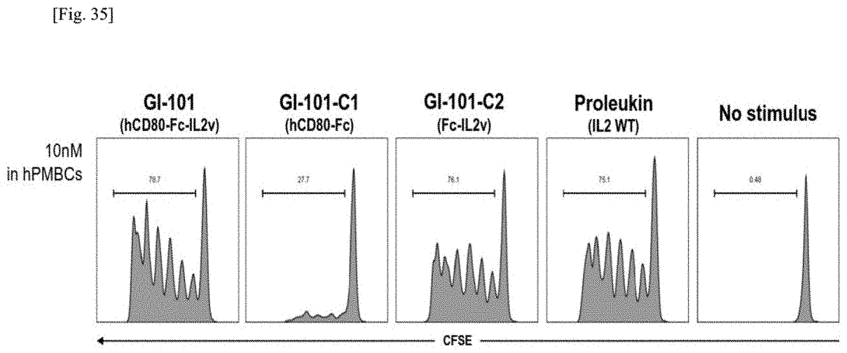

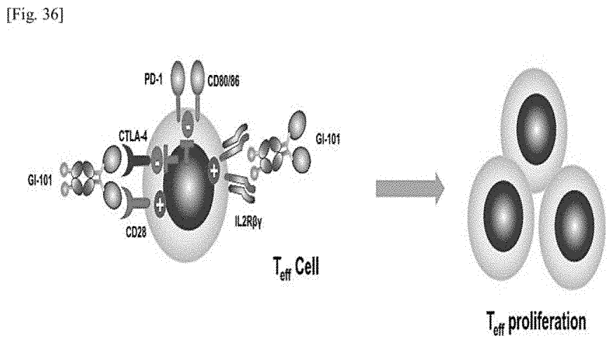

As a result, it was found that GI101 activated proliferation of CD8+ T cells in vitro to a similar extent to the wild-type IL-2 Proleukin (FIGS. 35 and 36).

### EXPERIMENTAL EXAMPLE 11

### Identification of Effect of GI101 and GI102 on Proliferation of CD8+ T cells

Human PBMCs were purchased from Allcells (Lot #3014928, USA). 1M CellTrace CFSE dye was used, which was reacted with the human PBMCs under a light-blocking condition at room temperature for 20 minutes. The cells were labeled with CFSE by being reacted with 1 μM CellTrace CFSE dye at 37° C. for 20 minutes. CFSE not bound to the cells was removed by being reacted for 5 minutes with culture medium having a 5-fold volume of the staining reaction solution and then by being centrifuged at 1,300 rpm for 5 minutes. The CFB-labeled PBMCs were resuspended in the culture medium (RPMI1640 medium containing 10% FBS, 10 mM HEPES, 100 U/ml penicillin/streptomycin, 1 mM sodium pyruvate, 55 μM 2-mercaptoethanol, 1 mM non-essential amino acid, and 2 mM L-glutamine), and then added to a 96-well plate at 1×10⁵ cells per well.

Thereafter, the CFB-labeled PBMCs were subjected to treatment with 1 μg/ml of anti-CD3ε antibody (OKT3, eBioscience, USA), and GI101, GI101C1, GI101C2, or Proleukin (Novartis), and incubation was performed in a 5% CO₂ incubator at 37° C. for 7 days. Here, the cells were subjected to treatment with GI101, GI101C1, GI101C2, and IL-2 at a concentration of 10 μM.

The incubated cells were examined for their degree of proliferation by measuring, with FACS analysis using anti-human CD4-PE antibody (BioLegend, USA), anti-human CD8-PE/Cy7 antibody (BioLegend, USA), and anti-human FoxP3-APC antibody (BioLegend, USA), a proportion of CD8+ T cells that had not been labeled with CFSE.

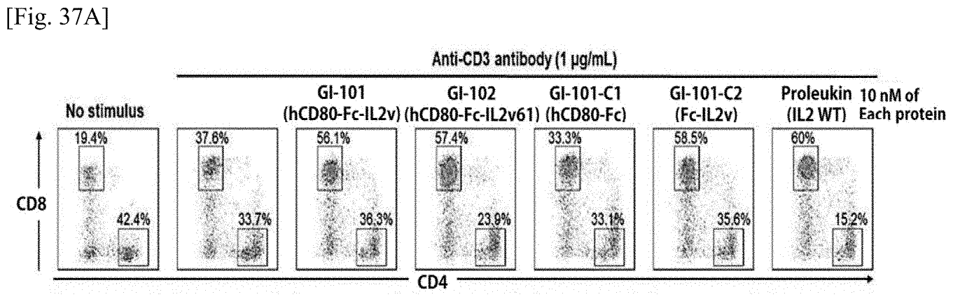

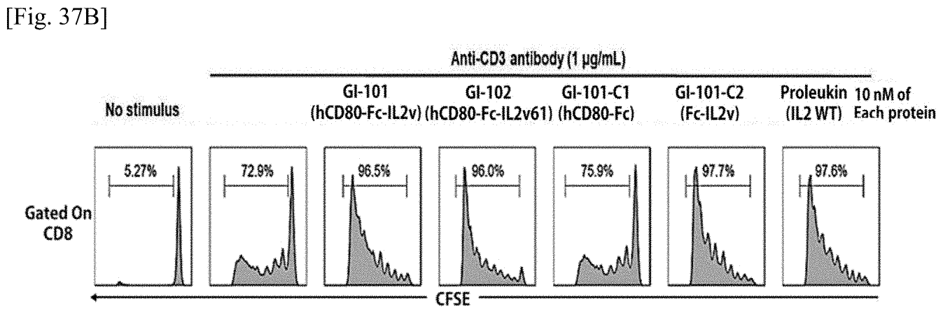

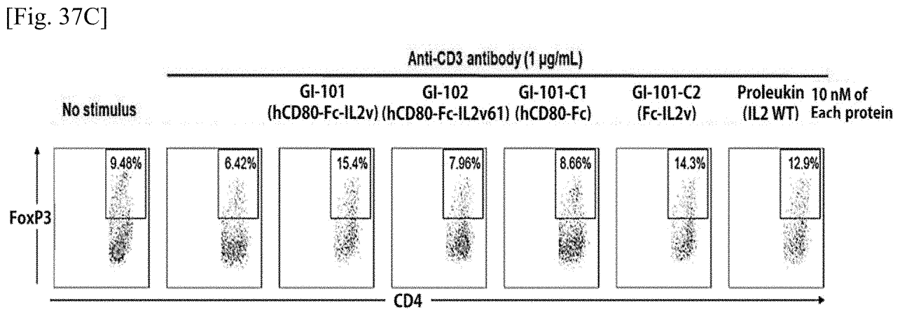

As a result, the GI101, GI102_M61, GI101C2, and Proleukin treatment groups exhibited a significant increase in proportion of CD8+ T cells, as compared with the control group (no stimulus), the anti-CD3 antibody alone treatment group, and the GI101C1 treatment group. In addition, as compared with the negative control group (no stimulus) and the anti-CD3 alone treatment group, the GI101, GI101C2, and Proleukin treatment groups exhibited a significant increase in proliferation of CD4+/FoxP3+ Treg cells, whereas the GI102 and GI101C1 treatment groups did not exhibit a significant increase in proliferation of CD4+/FoxP3+ Treg cells (FIGS. 37A-37C).

Experimental Example 12. Identification of effect of GI101 or GI101w on proliferation of CD8+ T cells and NK cells 7-week-old C57BL/6 mice purchased from Orient Bio (Busan, Korea) were divided into 3 groups, each group containing 3 mice, and PBS, GI101, or GI101w was injected intraperitoneally thereinto. Here, GI101 and GI101w were respectively prepared to be at 40.5 μg in 200 μl of PBS, and injected intraperitoneally thereinto. Five days after the injection, the spleens were removed from the mice of each group. The cells were isolated therefrom, and the total number of cells was measured using a hematocytometer. Splenocytes were examined for proportions of CD8+ T cells and NK cells therein, with FACS analysis using staining with APC-CD3c antibody (Biolegend; 145-2C11), PE-NK1.1 antibody (Biolegend; PK136), and Pacific blue-CD8α antibody (BD; 53-6.7). As such, the numbers of CD8+ T cells and NK cells present in the spleen were calculated.

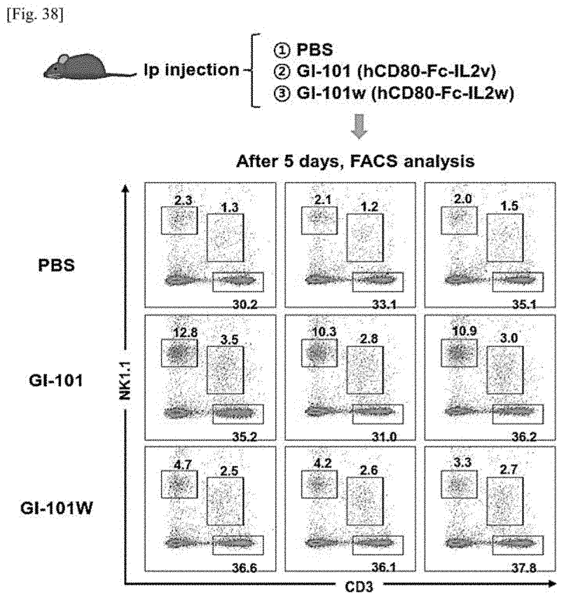

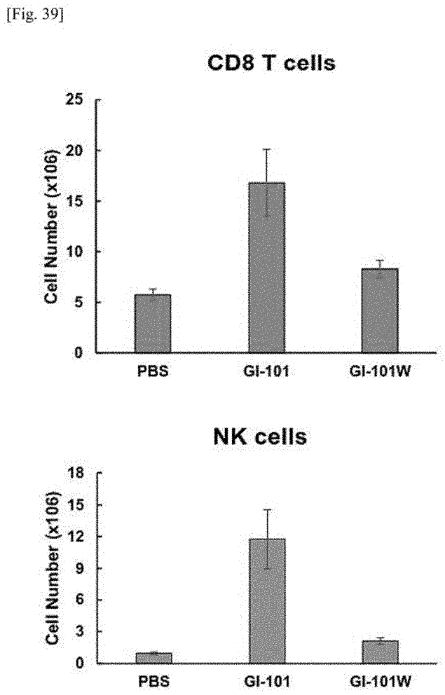

As a result, it was identified that GI101 activated proliferation of CD8+ T cells and NK cells in vivo as compared with GI101w (FIGS. 38 and 39).

### EXPERIMENTAL EXAMPLE 13

### Identification of Effect of GI101 on Function of T Cells

An experiment was performed using a CTLA-4 blockade bioassay kit (Promega Cat. No. JA4005). The experiment is briefly described as follows. CTLA-4 effector cells kept in liquid nitrogen were thawed in a 37° C. constant temperature water bath for 3 minutes, and 0.8 ml of CTLA-4 effector cells were mixed well with 3.2 ml of pre-warmed assay buffer (90% RPMI+10% FBS). Then, the mixture was added to a 96-well white cell culture plate (SPL, Cat. No. 30196) at 25 μl per well. Then, 25 μl of GI101 at various concentrations was added thereto. For a negative control, 25 μl of assay buffer was added thereto. Then, the white plat cell culture plate was covered and placed at room temperature until aAPC/Raji cells were prepared.

aAPC/Raji cells kept in liquid nitrogen were thawed in a 37° C. constant temperature water bath for 3 minutes, and 0.8 ml of aAPC/Raji cells were mixed well with 3.2 ml of pre-warmed assay buffer. Then, 25 μl of the mixture was added to the plate at per well, and reaction was allowed to proceed in a 5% CO₂ incubator at 37° C. for 16 hours. After the reaction was completed, the resultant was allowed to stand at room temperature for 15 minutes, and then the Bio-Glo reagent was added thereto while taking care to avoid bubbles. The Bio-Glo reagent was also added to three of the outermost wells and the wells were used as blanks to correct the background signal. Reaction was allowed to proceed at room temperature for 10 minutes, and then luminescence was measured with Cytation 3 (BioTek Instruments, Inc., Winooski, Vt., USA). Final data analysis was performed by calculating RLU (GI101-background)/RLU (no treatment-background).

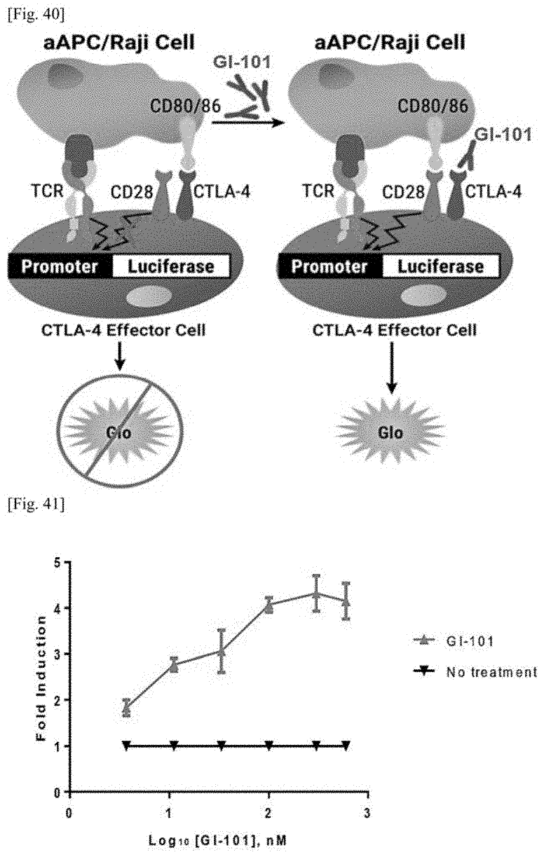

As a result, it was found that GI101 attached to CTLA-4 expressed on effector T cells, and activated the function of T cells rather than inhibiting the same (FIGS. 40 and 41).

### EXPERIMENTAL EXAMPLE 14

### Identification of Effect of mGI101 and mGI102 on Immune Cells

7-week-old C57BL/6 mice purchased from Orient Bio (Korea) were divided into 3 groups, each group containing 3 mice, and PBS, 3 mg/kg, 6 mg/kg, or 12 mg/kg of GI101, or 3 mg/kg, 6 mg/kg, or 12 mg/kg of mGI102 (mGI102-M61) was administered intravenously thereinto. On days 1, 3, 5, 7, and 14 after the injection, the spleens were removed from the mice of each group. Thereafter, for the spleen tissue, the numbers of effector CD8+ T cells, NK cells, and Treg cells were calculated with FACS analysis using respective antibodies, and proportions of effector CD8+ T cells and NK cells with respect to Treg cells were respectively calculated. The information on the antibodies used in each cell assay is as follows:

Effector CD8+ T cells: PB anti-mouse CD3ε antibody (Biolegend, #155612; KT3.1.1), FITC anti-mouse CD8α antibody (BD, #553031, 53-6.7), PE/Cy7 anti-mouse CD44 antibody (Biolegend, #103030; IM7), APC anti-mouse CD122 antibody (Biolegend, #123214; TM-I31)

NK cells: PB anti-mouse CD3ε antibody (Biolegend, #155612; KT3.1.1), PE anti-mouse NK-1.1 (Biolegend, #108708; PK136)

Treg cells: FITC anti-mouse CD3 antibody (Biolegend, #100204; 17A2), PB anti-mouse CD4 antibody (Biolegend, #100531; RM4-5), PE anti-mouse CD25 antibody (Biolegend, #102008; PC61), APC anti-mouse Foxp3 antibody (Invitrogen, #FJK-16s, 17-5773-82).

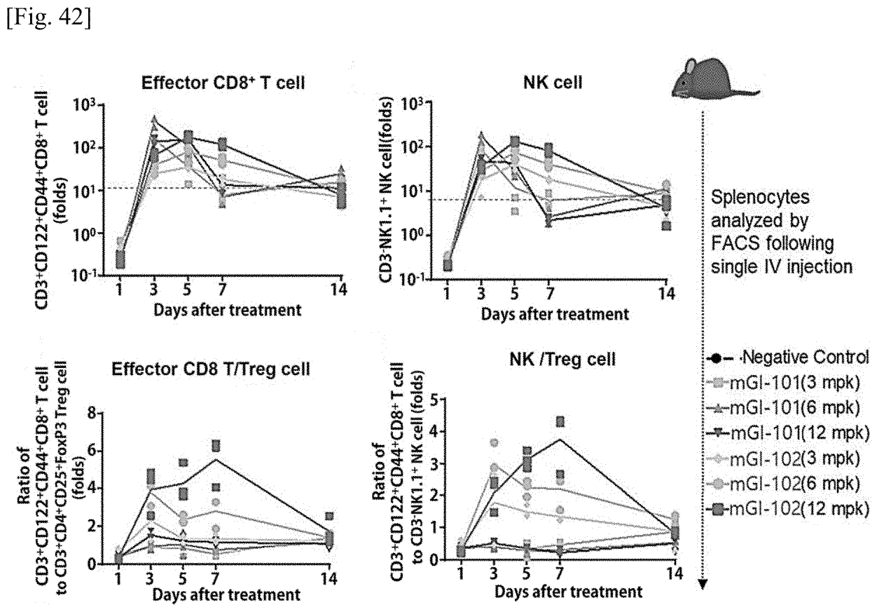

As a result, the group having received mGI101 or mGI102 (mGI102-M61) exhibited a significant increase in numbers of CD8+ T cells and NK cells at the time points from 3 days to 14 days after administration, as compared with the PBS administration group. In addition, it was found that the group having received mGI102 exhibited a significant increase in proportions of activated CD8+ T cells/Treg cells and NK cells/Treg cells at the time points from 3 days to 14 days after administration, as compared with the PBS administration group (FIG. 42).

IV. Identification of Anticancer Effect of Fusion Protein

### EXPERIMENTAL EXAMPLE 15

### Identification of Effect of GI101 on Cancer Cells Overexpressing PD-L1

NCl-H292 cancer cell line overexpressing PD-L1 was cultured for 3 hours in a culture medium containing 10 μg/ml Mitomycin C (Sigma), and then Mitomycin C was removed by washing with the culture medium. Thereafter, 5×10⁴ cells of the Mitomycin C-treated NCl-H292 cancer cell line were incubated with 1×10⁵ cells of human PBMCs in a 96-well plate. Here, treatment with 5 μg/ml of PHA (Sigma) was performed for T cell activity. In addition, GI101C1 and GI101 at a concentration of 50 nM were reacted with IgG1-Fc (Biolegend) or abatacept (=Orencia; Bristol-Myers Squibb) at a concentration of 50 nM for 30 minutes at 4° C., and then the resultant was used to treat the NCl-H292 cancer cells. After 3 days, the supernatant of the cell incubate was collected and the amount of IFN-γ was quantified using an ELISA kit (Biolegend).

As a positive control group, human PBMCs stimulated with PHA in the absence of the Mitomycin C-treated NCl-H292 cancer cell line were used; and as a negative control group, human PBMCs stimulated with PHA in the presence of the Mitomycin C-treated NCl-H292 cancer cell line was used. An experimental method using the IFN-γ ELISA kit was carried out in the same manner as in Experimental Example 9.3.

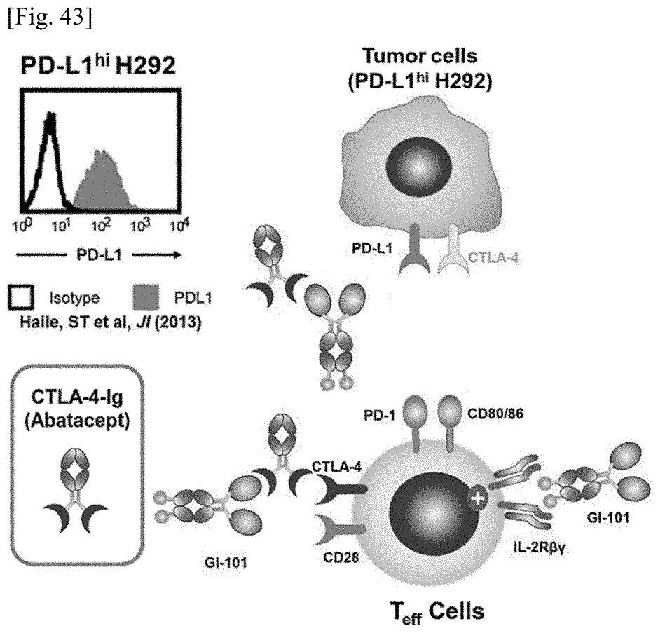

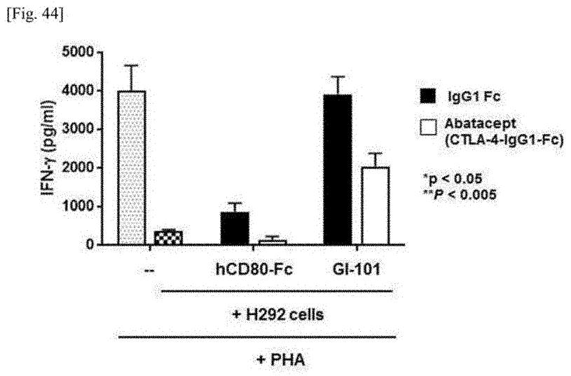

As a result, GI101 effectively activated the immune response that had been inhibited by the cancer cell line overexpressing PD-L1. In addition, it was discovered that GI101 inhibited signaling of CTLA-4 expressed on effector T cells (FIGS. 43 and 44).

### EXPERIMENTAL EXAMPLE 16

### Identification of Anticancer Effect of GI101 in Mouse-Derived Colorectal Cancer Cell-Transplanted Mice

5×10⁶ cells/0.05 ml of mouse-derived CT-26 cancer cell line was mixed with 0.05 ml Matrigel matrix phenol red-free (BD), and transplantation of 0.1 ml of the mixture was performed by subcutaneous administration in the right dorsal region of 6-week-old female BALB/c mice (Orient Bio). A certain period of time after the cancer cell transplantation, the tumor volume was measured and subjects that reached about 80 mm³ to 120 mm³ were separated. Then, the subjects were intravenously administered with 0.1 ml of GI101. A total of three administrations were given once every three days after the first administration, and PBS was given to a negative control group. The tumor size was measured daily to identify an anticancer effect.

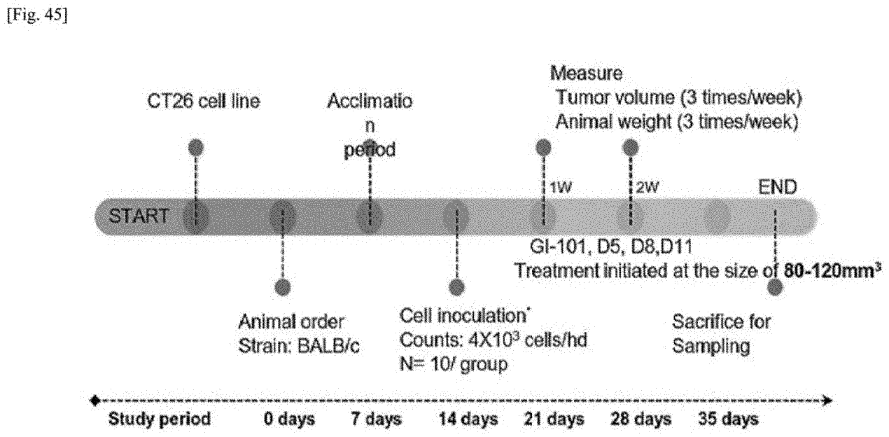

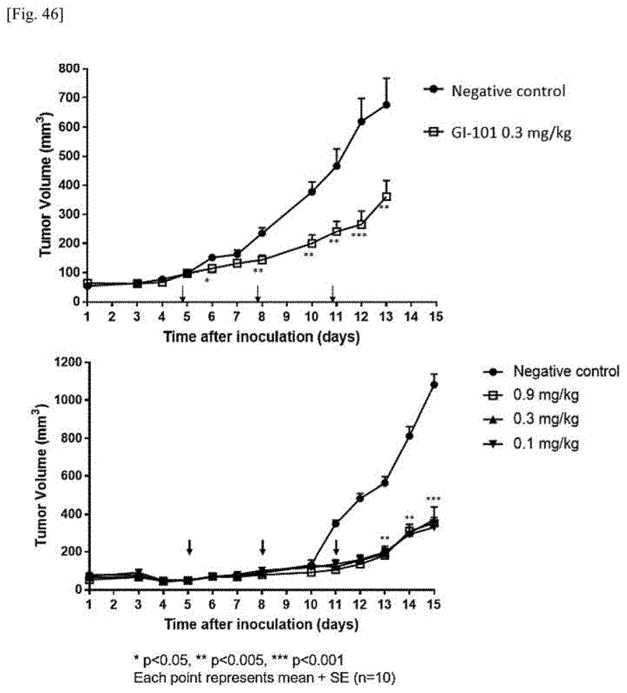

As a result, it was observed that the CT-26 cancer cell line-transplanted mice treated with GI101 exhibited a remarkable decrease in tumor size as compared with the negative control group (FIGS. 45 and 46).

### EXPERIMENTAL EXAMPLE 17

### Identification of Anticancer Effect of mGI101 in Mouse-Derived Melanoma-Transplanted Mice

C57BL/6 mice (female, 7-week-old) acquired from Orient Bio were subjected to an acclimation period of 7 days. Then, 5×10⁶ cells of B 16F10 cancer cell line (ATCC, USA) were mixed with 0.05 ml of Matrigel matrix phenol red-free (BD), and allotransplantation of the mixture was performed by subcutaneous administration at 0.1 ml in the right dorsal region of the mice. A certain period of time after the cancer cell transplantation, the tumor volume was measured and subjects that reached about 50 mm³ to 120 mm³ were selected, and then the selected mice were grouped evenly based on tumor size and body weight, each group containing 10 mice.

Thereafter, using a disposable syringe (31G, 1 mL), hIgG4 was administered at a dose of 4 mg/kg to a negative control group, and an anti-PD-1 antibody was administered at a dose of 5 mg/kg to a positive control group. For experimental groups, mGI101 at a dose of 1 mg/kg or 4 mg/kg was administered intravenously thereto. Additionally, groups having received mGI101 at a dose of 4 mg/kg and an anti-PD-1 antibody at a dose of 5 mg/kg were also set as experimental groups. A total of three administrations were given once every three days after the first administration. The tumor size was measured daily.

As a result, the initial tumor volume of all groups was 90 mm³, and standard error (S.E.) of each group was 5 mm³ to 6 mm³. In the negative control group, a change in tumor volume was observed during the experimental period, in which the tumor volume increased from 90 mm³ to 1,434 mm³ up to 15 days after the administration.

In the group having received mGI101 at a dose of 1 mg/kg, the tumor volume was observed to increase from 90 mm³ to 885 mm³ during the experimental period which is the same period as the negative control group, and a statistically significant inhibition of tumor growth was observed at some measurement time points (p-value: 0.5 on day 11, p-value<0.01 on day 7, p-value<0.001 on day 3). In the group having received mGI101 at a dose of 4 mg/kg, the tumor volume was observed to increase from 90 mm³ to 748 mm³ during the experimental period which is the same period as the negative control group, and a statistically significant inhibition of tumor growth was observed at some measurement time points (p-value: 0.5 on day 9, p-value<0.01 on days 7 and 11).

In addition, tumor growth inhibition rate was analyzed by using, as a reference, the group having received mIgG at a dose of 4 mg/kg and comparing this group with each of the other groups. In the group having received mGI101 at a dose of 1 mg/kg, growth inhibition rate of 36.5% was observed as compared with the negative control group, and no statistically significant difference (p-value: 0.5) was observed. In the group having received mGI101 at a dose of 4 mg/kg, a statistically significant (p-value: 0.5) tumor growth inhibition rate was observed as compared with the negative control group. A total of two administrations were given once every three days after the first administration. The tumor size was measured daily.

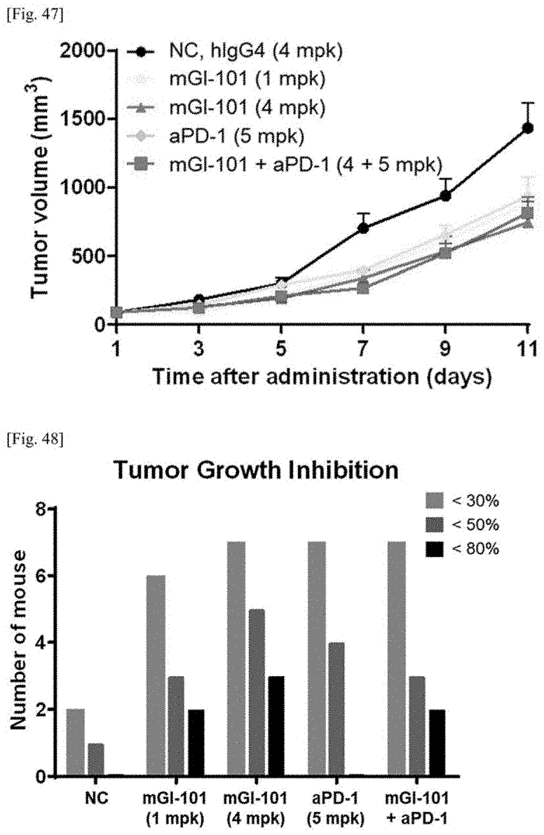

Through this, it was found that in tumor growth inhibitory efficacy test for B16F10, a melanoma allotransplanted into C57BL/6 mice, mGI101 had an effect of inhibiting tumor growth in a dose-dependent manner (FIGS. 47 and 48).

### EXPERIMENTAL EXAMPLE 18

### Identification of Anticancer Effect of mGI101 in Mouse-Derived Colorectal Cancer Cell-Transplanted Mice

BALB/c mice (female, 7-week-old) acquired from Orient Bio were subjected to an acclimation period of 7 days. Then, 5×10⁶ cells of CT-26 cancer cell line (ATCC, USA) were mixed with 0.05 ml of Matrigel matrix phenol red-free (BD), and allotransplantation of the mixture was performed by subcutaneous administration at 0.1 ml in the right dorsal region of the mice. A certain period of time after the cancer cell transplantation, the tumor volume was measured and subjects that reached about 28 mm³ were selected, and then the selected mice were grouped evenly based on tumor size and body weight, each group containing 10 mice. Thereafter, using a disposable syringe (31G, 1 mL), hIgG4 was administered at a dose of 6 mg/kg to a negative control group. For experimental groups, mGI101 at a dose of 3 mg/kg, 6 mg/kg, or 12 mg/kg was administered intravenously thereto. A total of three administrations were given once every three days after the first administration. The tumor size was measured daily.

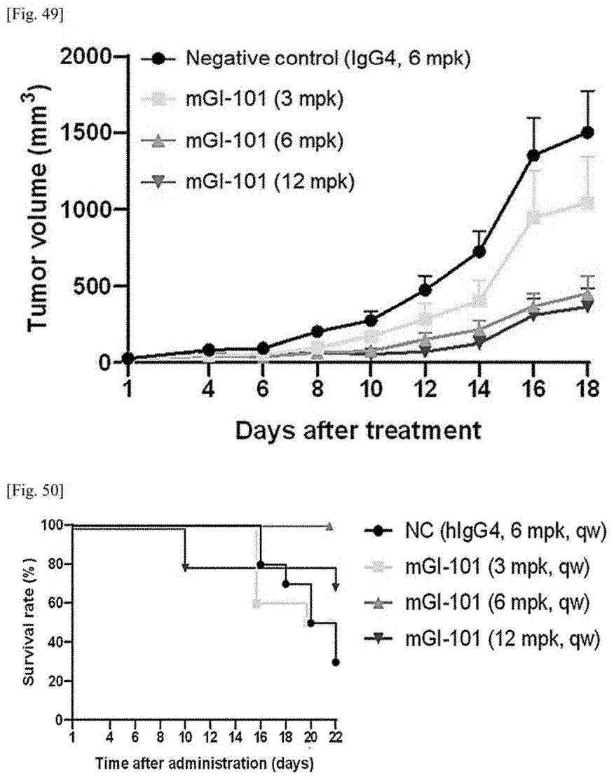

As a result, it was found that the experimental group having received mGI101 at a dose of 6 mg/kg or 12 mg/kg mGI101 exhibited significant inhibition of tumor growth at some measurement time points and at the end of the test, as compared with the negative control group (FIG. 49). In addition, as a result of measuring a survival rate, it was found that the experimental group having received mGI101 at a dose of 6 mg/kg exhibited significant improvement at some measurement time points and at the end of the test, as compared with the negative control group (FIG. 50).

### EXPERIMENTAL EXAMPLE 19

### Identification of Anticancer Effect of GI101 in Mice Transplanted with Mouse-Derived Colorectal Cancer Cells

### EXPERIMENTAL EXAMPLE 19.1

### Identification of Tumor Inhibitory Effect

BALB/c mice (female, 7-week-old) acquired from Orient Bio were subjected to an acclimation period of 7 days. Then, 5×10⁶ cells of CT-26 cancer cell line (ATCC, USA) were suspended in 0.1 ml PBS, and allotransplantation of the suspension was performed by subcutaneous administration at 0.1 ml in the right dorsal region of the mice. A certain period of time after the cancer cell transplantation, the tumor volume was measured and subjects that reached about 50 mm³ to 200 mm³ were selected, and then the selected mice were grouped evenly based on tumor size and body weight, each group containing 10 mice. Thereafter, using a disposable syringe (31G, 1 mL), no drug was administered to a negative control group, and an anti-PD-1 antibody at a dose of 5 mg/kg, or an anti-PD-1 antibody at a dose of 5 mg/kg and an anti-CTLA-4 antibody at a dose of 5 mg/kg were administered intravenously to positive control groups. For experimental groups, GI101 at a dose of 0.1 mg/kg or 1 mg/kg was administered intravenously thereto. A total of three administrations were given once every three days after the first administration. The tumor size was measured daily.

As a result, in the CT-26 cancer cell line-transplanted mice, all groups having received anti-PD-1 antibody, anti-PD-1 antibody and anti-CTLA-4 antibody, or GI101 at a dose of 0.1 mg/kg or 1 mg/kg exhibited significant inhibition of tumor growth, as compared with the negative control. In particular, the experimental group having received GI101 at a dose of 0.1 mg/kg exhibited a significant tumor inhibitory effect, as compared with the anti-PD-1 antibody treatment group (*p<0.05) (FIG. 51).

### EXPERIMENTAL EXAMPLE 19.2

### Immune Cell Analysis in Cancer Tissue

The mice of each group in Experimental Example 19.1 were sacrificed when the tumor volume reached an average of 200 mm³, and cancer tissues were collected. Thereafter, the cancer tissues were separated to a single-cell level to analyze immune cells therein, and then FACS analysis was performed on immune cells in the cancer tissues using the following antibodies: Anti-mouse-CD3 (Biolegend, Cat. No. 100320), Anti-mouse-CD4 (Biolegend, Cat. No. 100526), Anti-mouse-CD8 (Biolegend, Cat. No. 100750), Anti-mouse-FoxP3 (eBioscience, Cat. No. 12-5773-82), Anti-mouse-CD25 (Biolegend, Cat. No. 102049), Anti-mouse-CD44 (eBioscience, Cat. No. 61-0441-82), Anti-mouse-PD-1 (Biolegend, Cat. No. 135218), Anti-mouse-IFN-gamma (Biolegend, Cat. No. 505832), Anti-mouse-CD49b (Biolegend, Cat. No. 108906), Anti-mouse-H2 (Invitrogen, Cat. No. A15443), Anti-mouse-CD11c (Biolegend, Cat. No. 117343), Anti-mouse-CD80 (eBioscience, Cat. No. 47-4801-82), Anti-mouse-CD86 (Biolegend, Cat. No. 104729), Anti-mouse-F4/80 (eBioscience, Cat. No. 47-4801-82), and Anti-mouse-CD206 (eBioscience, Cat. No. 17-2061-80).

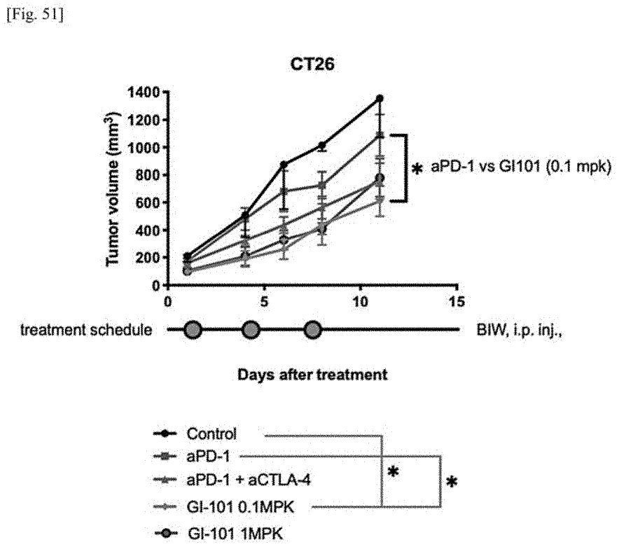

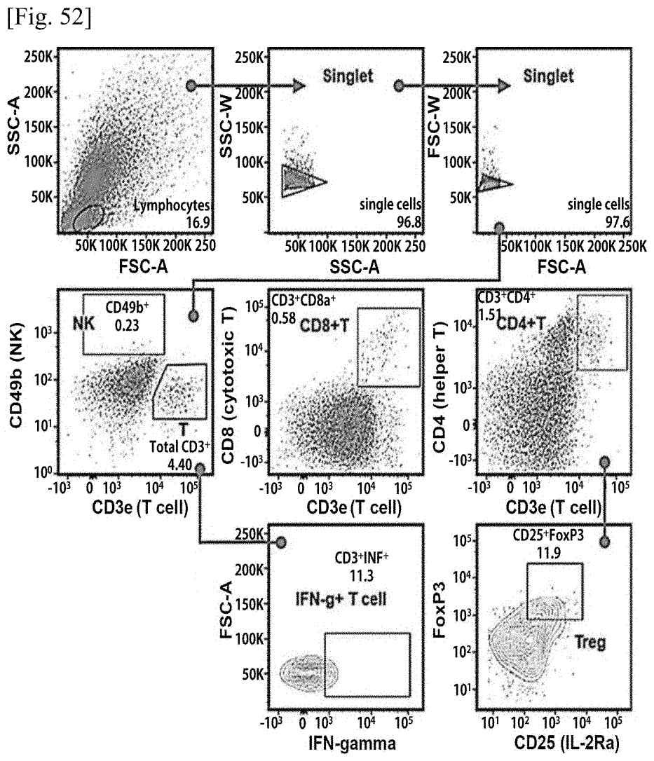

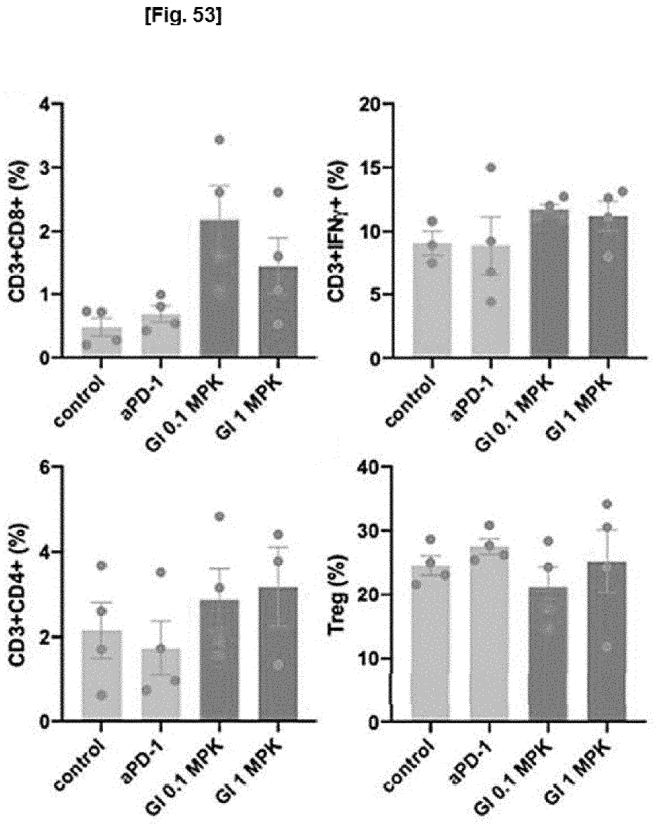

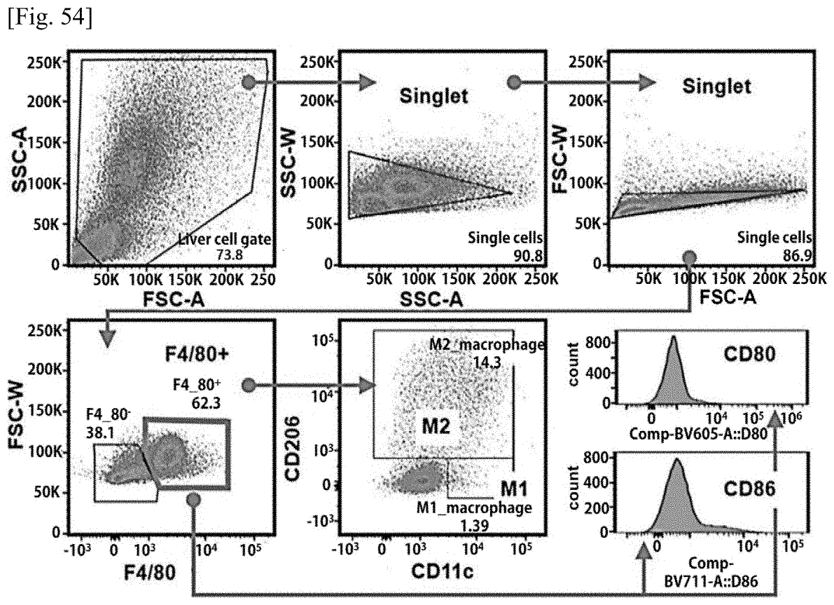

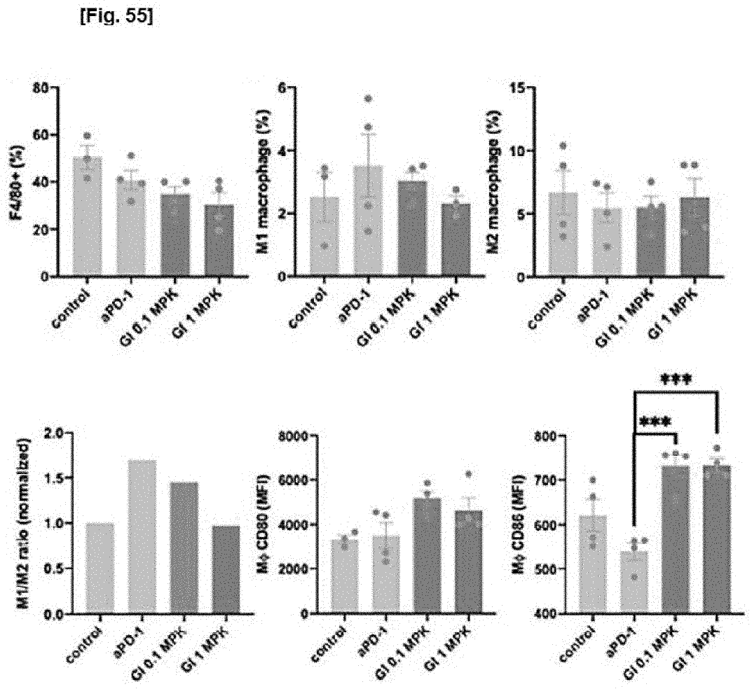

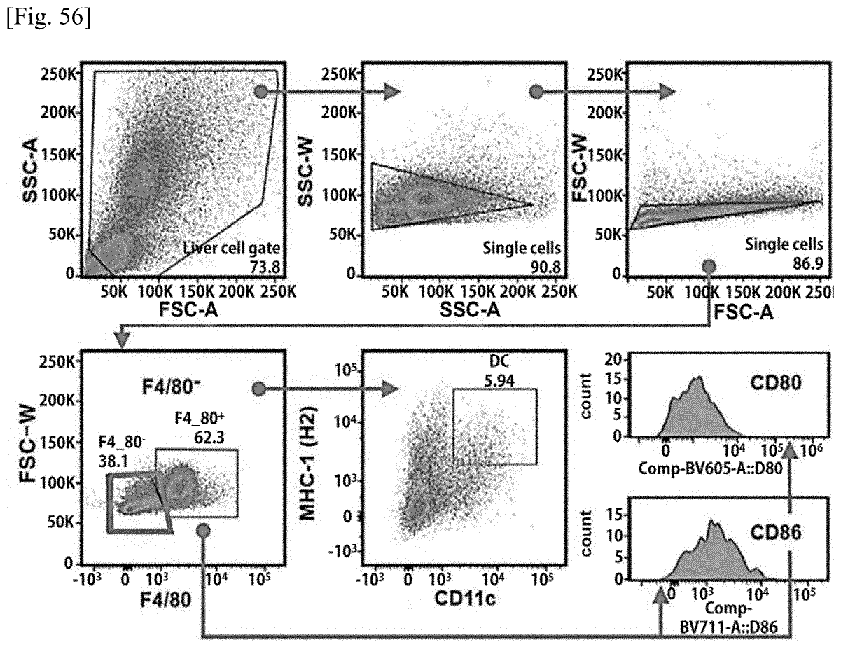

As a result, the experimental group having received GI101 at a dose of 0.1 mg/kg exhibited a significant increase in CD8+ T cells, as compared with the positive control group having received anti-PD-1 antibody alone at a dose of 5 mg/kg (*p<0.05, FIGS. 52 and 53). Furthermore, all experimental groups having received GI101 exhibited a significantly increased level of expression of IFN-γ in T cells, as compared with the negative control group (*p<0.05, FIGS. 52 and 53). In addition, the experimental group having received GI101 at a dose of 0.1 mg/kg exhibited an increase in M1 macrophages as compared with the negative control group and the positive control group having received anti-PD-1 antibody alone (FIGS. 54 and 55). In addition, all experimental groups having received GI101 exhibited an increased level of CD86 expression in macrophages and dendritic cells (*p<0.05, FIGS. 54 to 57).

### EXPERIMENTAL EXAMPLE 20

### Identification of Anticancer Effect of GI101 in Mice Transplanted with Mouse-Derived Lung Cancer Cells

### EXPERIMENTAL EXAMPLE 20.1

### Identification of Tumor Inhibitory Effect

C57BL/6 mice (female, 7-week-old) acquired from Orient Bio were subjected to an acclimation period of 7 days. Then, 5×10⁶ cells of LLC2 cancer cell line (ATCC, USA) were suspended in 0.1 ml PBS, and allotransplantation of the suspension was performed by subcutaneous administration at 0.1 ml in the right dorsal region of the mice. A certain period of time after the cancer cell transplantation, the tumor volume was measured and subjects that reached about 50 mm³ to 200 mm³ were selected, and then the selected mice were grouped evenly based on tumor size and body weight, each group containing 10 mice. Thereafter, using a disposable syringe (31G, 1 mL), no drug was administered to a negative control group, and an anti-PD-1 antibody at a dose of 5 mg/kg, or an anti-PD-1 antibody at a dose of 5 mg/kg and an anti-CTLA-4 antibody at a dose of 5 mg/kg were administered intravenously to positive control groups. For experimental groups, GI101 at a dose of 0.1 mg/kg or 1 mg/kg was administered intravenously thereto. A total of three administrations were given once every three days after the first administration. The tumor size was measured daily.

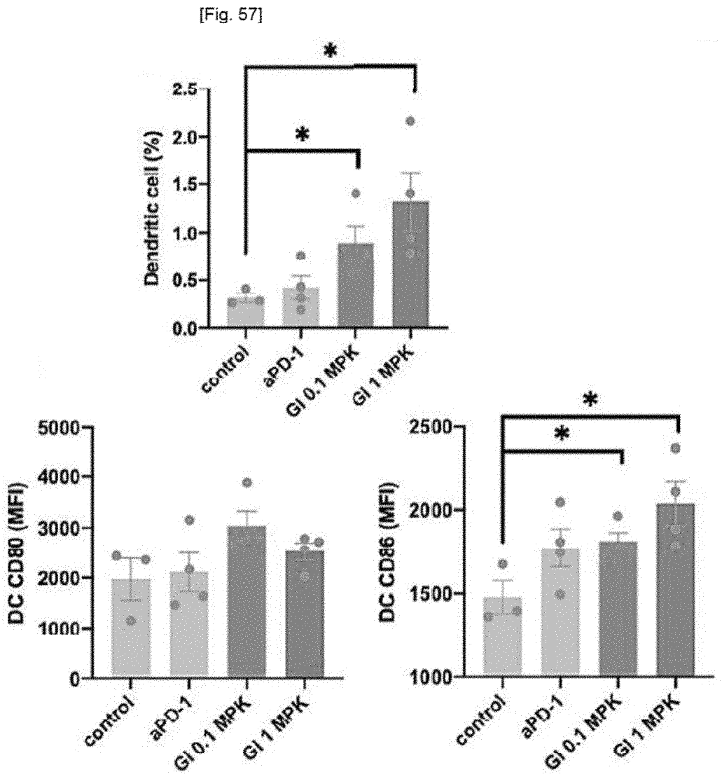

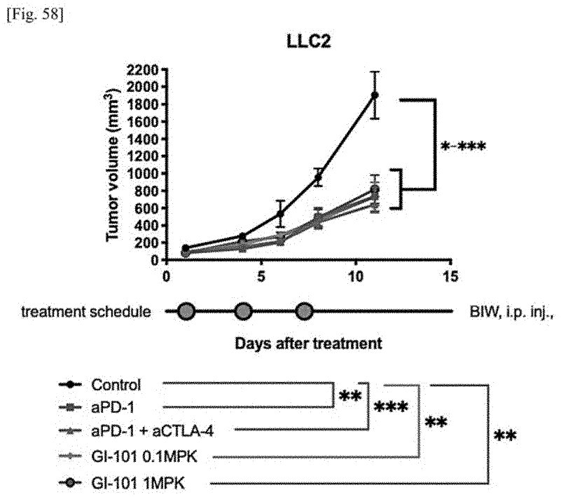

As a result, all experimental groups exhibited a significant tumor inhibitory effect, as compared with the negative control group (*p<0.05) (FIG. 58).

### EXPERIMENTAL EXAMPLE 20.2

### Immune Cell Analysis in Cancer Tissue

The mice of each group in Experimental Example 20.1 were sacrificed when the tumor volume reached an average of 200 mm³, and cancer tissues were collected. Thereafter, FACS analysis was performed in the same manner as Experimental Example 19.2 to analyze immune cells in the cancer tissues.

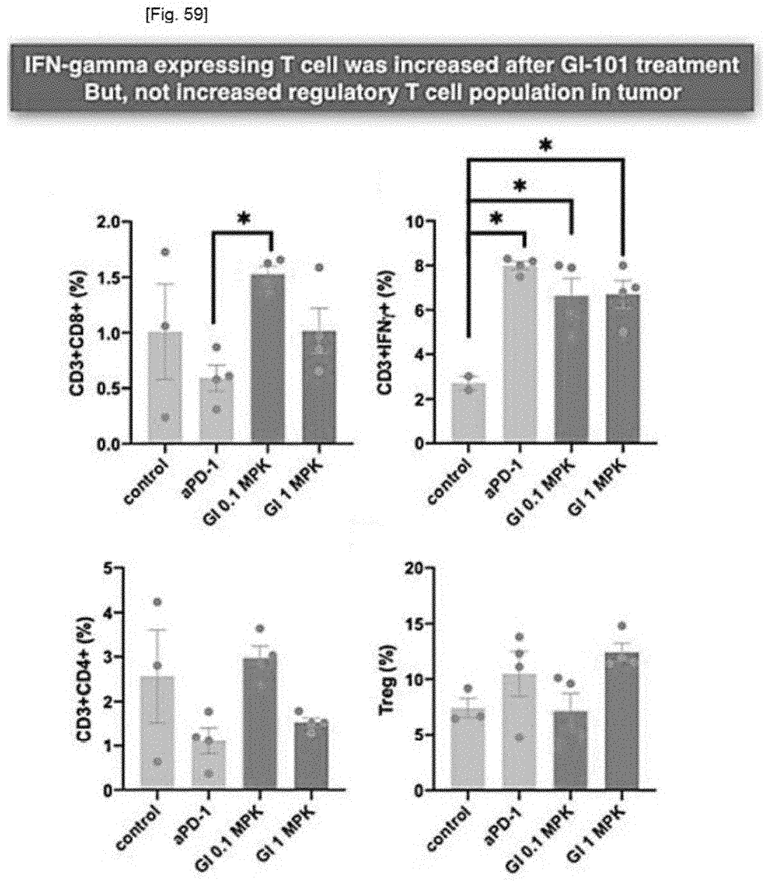

As a result, the experimental group having received GI101 at a dose of 0.1 mg/kg exhibited a significant increase in CD8+ T cells, as compared with the positive control group having received anti-PD-1 antibody alone (*p<0.05, FIG. 59). Furthermore, all experimental groups having received GI101 exhibited a significantly increased level of expression of IFN-γ, as compared with the negative control group (*p<0.05, FIG. 59). In addition, all experimental groups having received GI101 exhibited an increased level of CD86 expression in macrophages and dendritic cells (*p<0.05, FIGS. 59 to 61).

### EXPERIMENTAL EXAMPLE 21

### Identification of Anticancer Effect of mGI102-M61 in Mice Transplanted with Mouse-Derived Colorectal Cancer Cells

BALB/c mice (female, 7-week-old) acquired from Orient Bio were subjected to an acclimation period of 7 days. Then, 5×10⁶ cells of CT-26 cancer cell line (ATCC, USA) were mixed with 0.05 ml of Matrigel matrix phenol red-free (BD), and allotransplantation of the mixture was performed by subcutaneous administration at 0.1 ml in the right dorsal region of the mice. A certain period of time after the cancer cell transplantation, the tumor volume was measured and subjects that reached about 28 mm³ were selected, and then the selected mice were grouped evenly based on tumor size and body weight, each group containing 10 mice. Thereafter, using a disposable syringe (31G, 1 mL), hIgG4 was administered at a dose of 6 mg/kg to a negative control group. For experimental groups, mGI102-M61 at a dose of 3 mg/kg, 6 mg/kg, or 12 mg/kg was administered intravenously thereto. A total of three administrations were given once every three days after the first administration. The tumor size was measured daily.

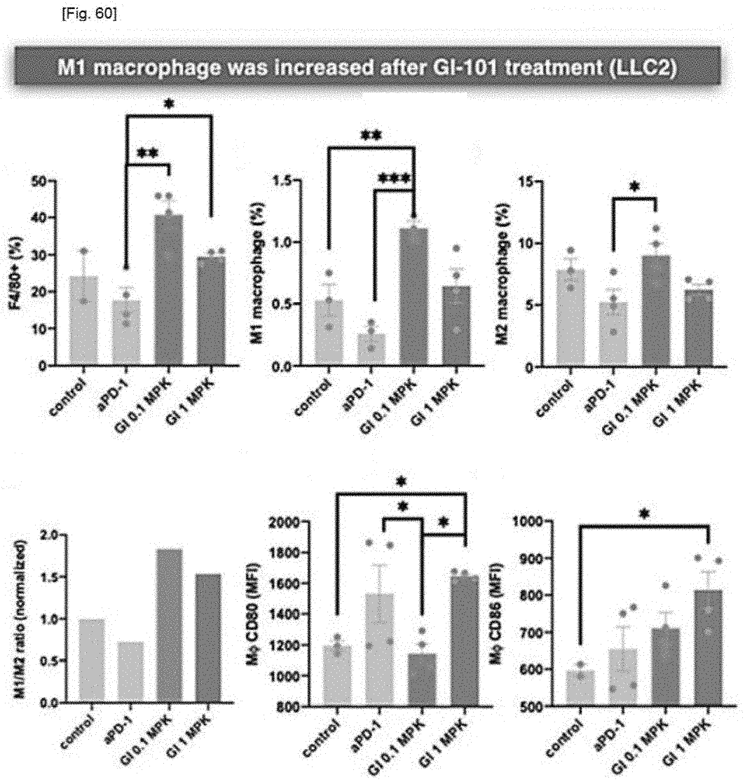

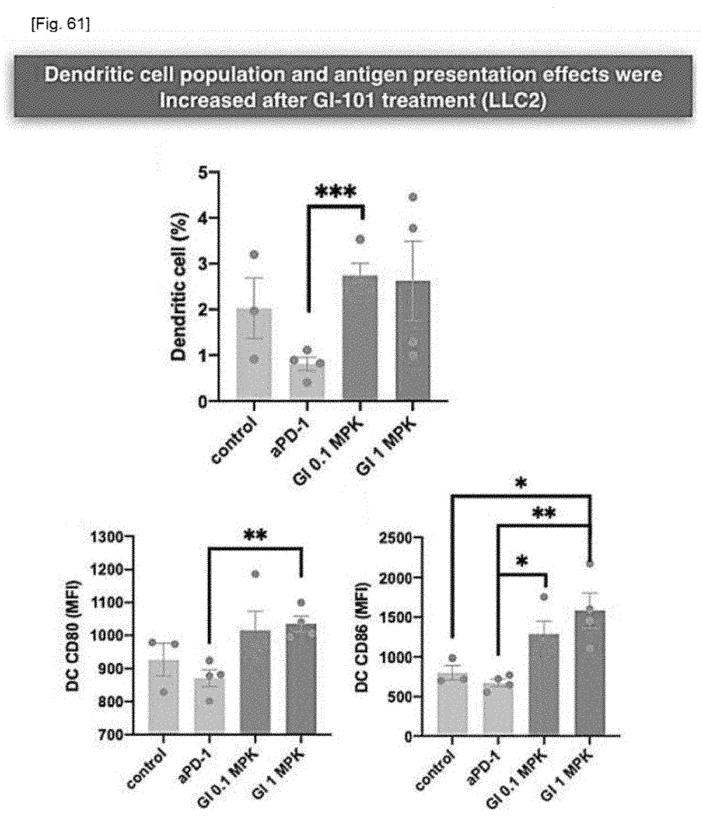

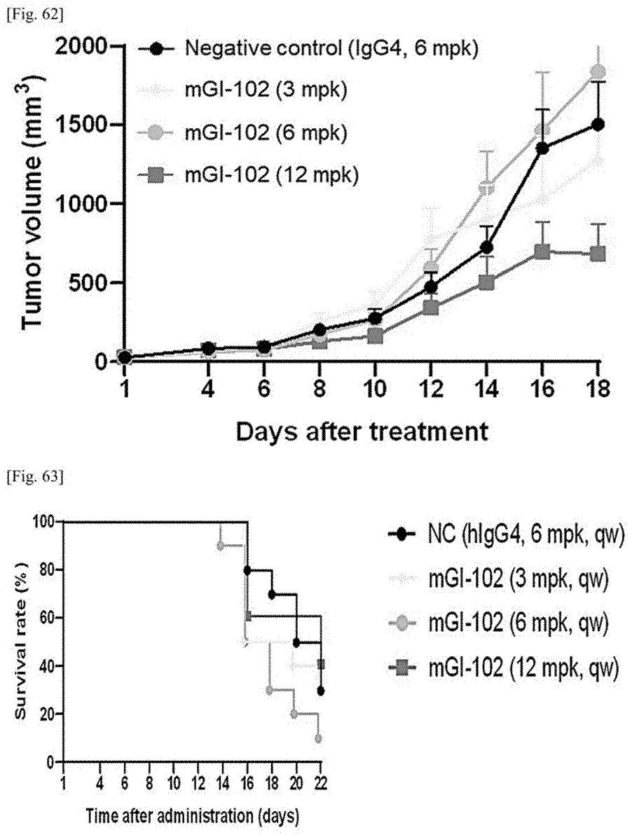

As a result, it was identified that the experimental group having received mGI102-M61 at a dose of 12 mg/kg exhibited significant inhibition of tumor growth at some measurement time points and at the end of the test, as compared with the negative control group (FIG. 62). In addition, as a result of measuring a survival rate, it was identified that the experimental group having received mGI102-M61 at a dose of 12 mg/kg exhibited significant improvement at some measurement time points and at the end of the test, as compared with the negative control group (FIG. 63).

### EXPERIMENTAL EXAMPLE 22

### Identification of Anticancer Effect of mGI101 in Mice Transplanted with Mouse-Derived Colorectal Cancer Cells

BALB/c mice (female, 7-week-old) acquired from Orient Bio were subjected to an acclimation period of 7 days. Then, 5×10⁶ cells of CT-26 cancer cell line (ATCC, USA) were mixed with 0.05 ml of Matrigel matrix phenol red-free (BD), and allotransplantation of the mixture was performed by subcutaneous administration at 0.1 ml in the right dorsal region of the mice. A certain period of time after the cancer cell transplantation, the tumor volume was measured and subjects that reached about 200 mm³ to 250 mm³ were selected, and then the selected mice were grouped evenly based on tumor size and body weight, each group containing 10 mice.

Thereafter, using a disposable syringe (31G, 1 mL), hIgG4 was administered at a dose of 4 mg/kg to a negative control group. For experimental groups, mGI101 at a dose of 1 mg/kg, 4 mg/kg, or 6 mg/kg was administered intravenously thereto. Additionally, groups having received mCD80 at 4.9 mg/kg or Fc-IL-2v (GI101C2) at 2.8 mg/kg were set as control groups. In addition, a group having simultaneously received mCD80 at 4.9 mg/kg and Fc-IL-2v (GI101C2) at 2.8 mg/kg was set as a control group.

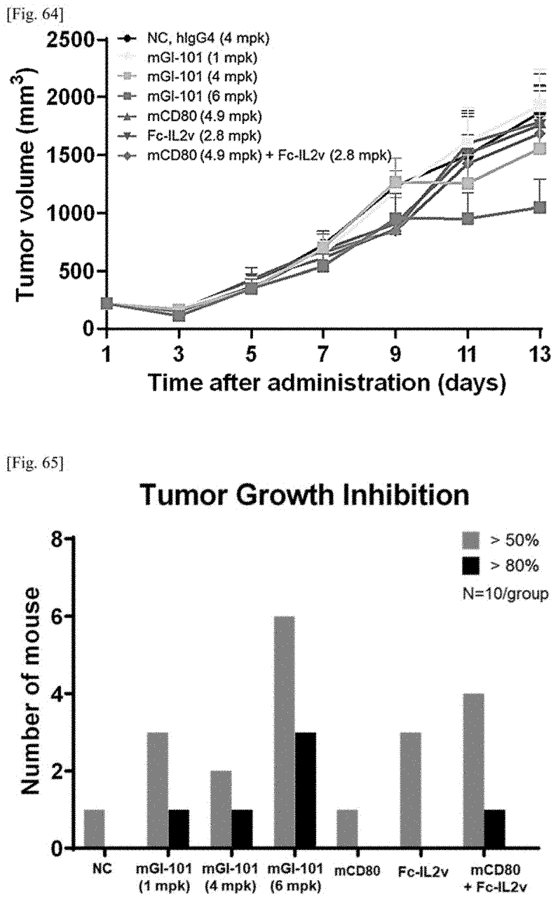

In tumor volume measurement, it was identified that the group having received mGI101 at a dose of 6 mg/kg exhibited significant inhibition at some measurement time points and at the end of the test, as compared with the negative control. An excellent tumor growth inhibition rate was observed as compared with the group having received a combination of mCD80 and Fc-IL-2v (GI101C2) (FIGS. 64 and 65).

In conclusion, in the tumor growth-inhibitory efficacy test on BALB/c mice allotransplanted with CT-26, a BALB/c mouse-derived colorectal cancer cell line, it was demonstrated that the test substance mGI101 had tumor inhibitory efficacy under this test condition as compared with mCD80 and IL-2v single preparations; and it was identified that mGI101 exhibited excellent anticancer efficacy as compared with the group having received a combination of mCD80 and IL-2v (FIGS. 64 and 65). In particular, the group having received mGI101 at a dose of 6 mg/kg exhibited significant inhibition of tumor size, as compared with the negative control group and the group having received a combination of mCD80 and Fc-IL2v (GI101C2).

V. Toxicity Evaluation of Fusion Protein

### EXPERIMENTAL EXAMPLE 23

### Toxicity Evaluation of GI101 Using Monkeys

### EXPERIMENTAL EXAMPLE 23.1

### Monkey Breeding and Drug Administration

In the present experiment, nine male Philippine monkeys (Cynomolgus monkeys) aged 2 to 3 years were used. The experiment was carried out in accordance with the “Act on Welfare and Management of Animals” in Japan and the “Guidance for Animal Care and Use” of Ina Research Inc. The experimental protocol was reviewed by the Institutional Animal Care and Use Committee (IACUC) of Ina Research Inc, and then approved by AAALAC International (Accredited Unit No. 001107).

The experiment was conducted from one day before drug administration up to 15 days after drug administration. Each monkey was observed around the cage, and the stool status was additionally checked. Body weights were measured using a digital scale (LDS-150H, Shimadzu Corporation) one day before drug administration, and on days 1, 8, and 15 after drug administration. In addition, the remaining amount of food was measured from one day before drug administration up to sacrifice of the monkeys.

Here, a disposable syringe (24G) was filled with the drug GI101, and a total of two administrations were given via an intravenous route, each administration being made at a rate of 0.17 ml/sec. GI101 was given twice, at a week's interval, at a dose of 5 mg/kg/day or 10 mg/kg/day. A control group was administered PBS (pH 7.4) in the same manner.

### EXPERIMENTAL EXAMPLE 23.2

### Clinical Observation, Identification of Changes in Body Weight and Food Intake

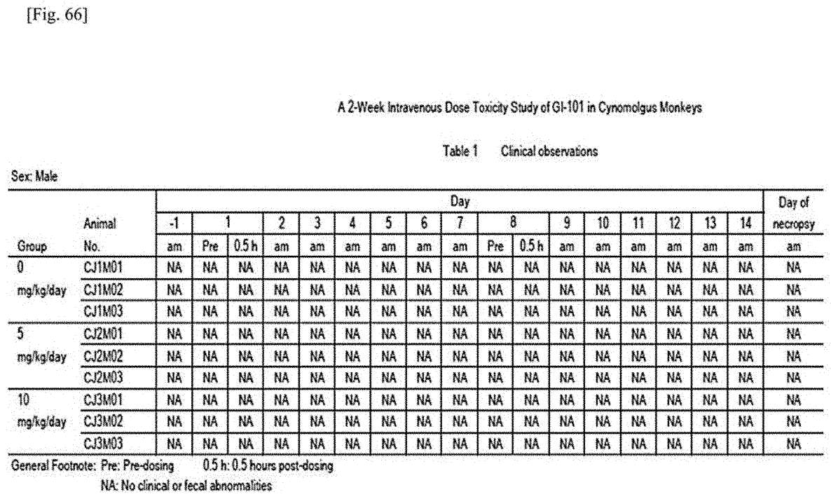

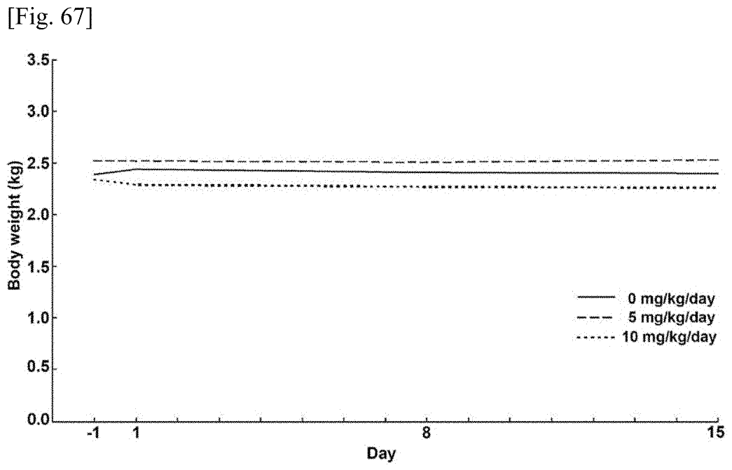

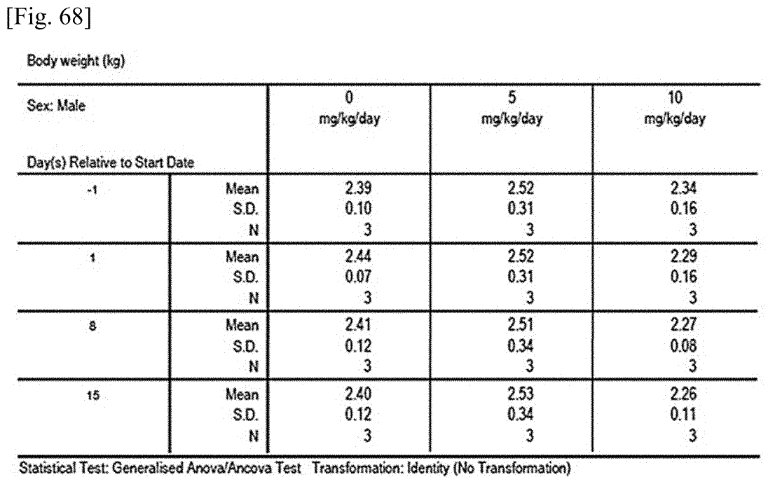

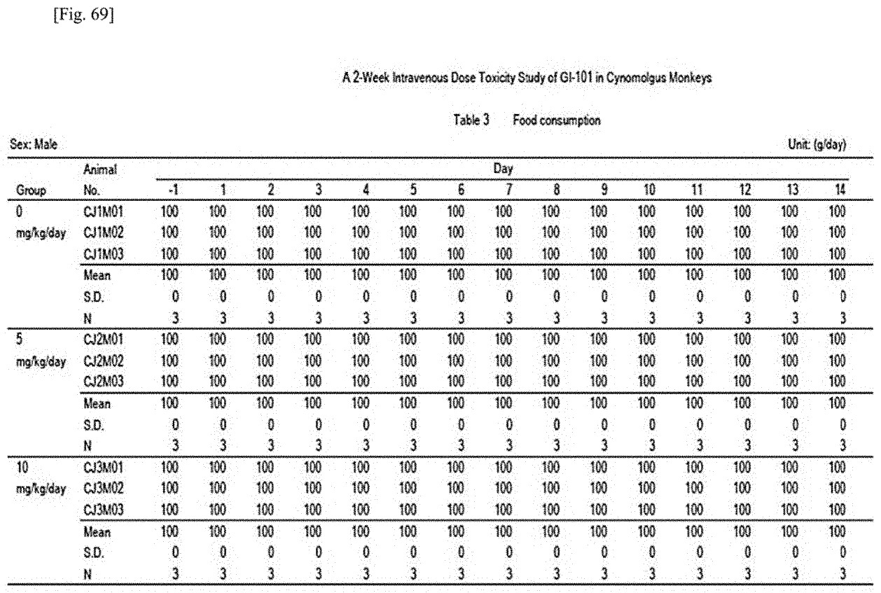

Clinical observation, and measurement of changes in body weight and food intake were performed from one day before drug administration up to days 1, 8, and 15 after drug administration. As a result, no toxicity was caused by GI101 (FIGS. 66 to 69).

### EXPERIMENTAL EXAMPLE 23.3

### Blood Analysis

Blood was collected from the monkeys in Experimental Example 23.1 one day before drug administration, and on days 1, 8, and 15 after drug administration. Here, the blood was collected via the femoral vein with a disposable syringe (22G). The collected blood was subjected to blood analysis using the Automated Hematology System XN-2000 (Sysmex Corporation) and the Automated Blood Coagulation Analyzer CA-510 (Sysmex Corporation) for the items listed in Table 2 below.

TABLE 2

| | | | | Equip- |
|---|---|---|---|---|
| Parameter | Abbr. | Unit | Method | ment |
| Complete blood | | | | |
| count | | | | |
| Red blood cell | RBC | 10⁶/μL | DC sheath-flow | XN-2000 |
| count | | | detection | |
| Hemoglobin | HGB | g/dL | SLS-hemoglobin | XN-2000 |
| concentration | | | | |
| Hematocrit | HCT | % | RBC pulse | XN-2000 |
| | | | height detection | |
| Mean corpuscular | MCV | fL | HCT/RBC | XN-2000 |
| volume | | | (×10⁴/μL) × 1000 | |
| Mean corpuscular | MCH | pg | HGB/RBC | XN-2000 |
| hemoglobin | | | (×10⁴/μL) × 1000 | |
| Mean corpuscular | MCHC | g/dL | HGB/HCT × 100 | XN-2000 |
| hemoglobin | | | | |
| concentration | | | | |
| Reticulocytes | RET % | % | Flow cytometry | XN-2000 |
| Ratio | | | | |
| Count | RET # | 10⁹/L | | |
| Platelet count | PLT | 10³/μL | Flow cytometry | XN-2000 |
| White blood cell | WBC | 10³/μL | Flow cytometry | XN-2000 |
| count | | | | |
| Differential white | Diff | % | Flow cytometry | XN-2000 |
| blood cells ^{a)}Ratio | WBC % | 10³/μL | | |
| Count | Diff | | | |
| | WBC # | | | |
| Coagulation tests | | | | |
| Prothrombin time | PT | s | Light scattering | CA-510 |
| | | | detection | |
| Activated partial | APTT | s | Light scattering | CA-510 |
| thromboplastin | | | detection | |

^{a)}Neutrophils (NEUtimeT), lymphocytes (LYMPH), monocytes (MONO), eosinophils (EO) and basophils (BASO)

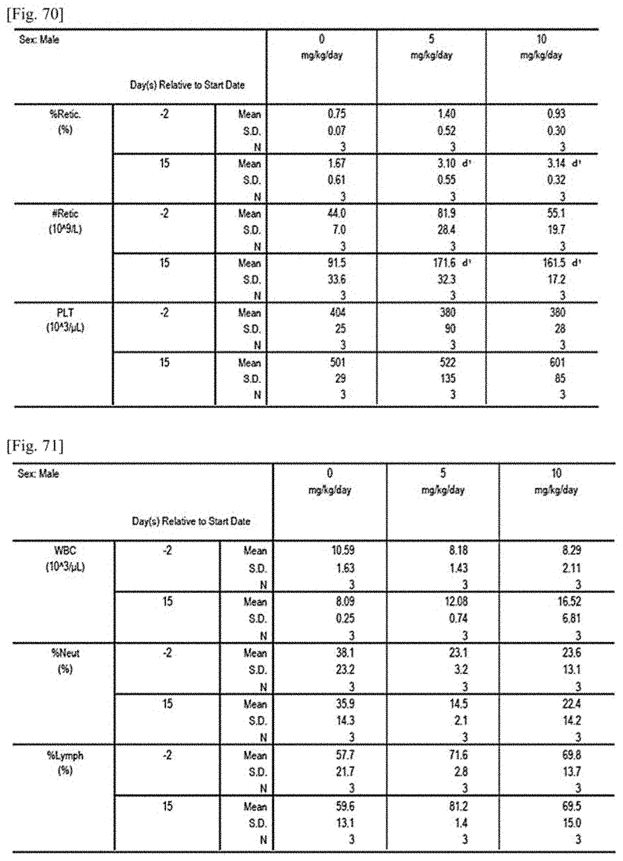

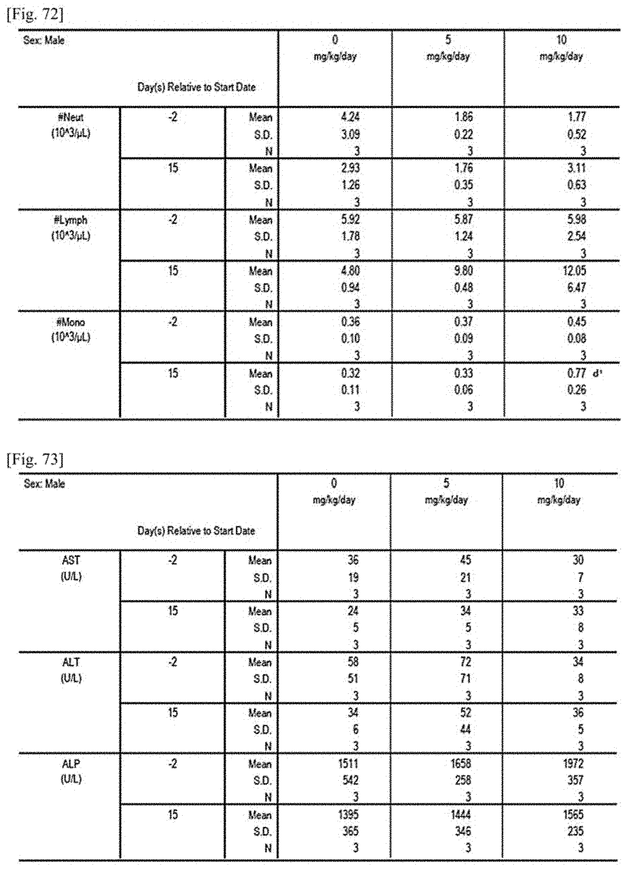

As a result, the group having received GI101 at a dose of 5 mg/kg/day or 10 mg/kg/day exhibited an increase in numbers of reticulocytes, leukocytes, and lymphocytes on day 15 (FIGS. 70 to 72).

### EXPERIMENTAL EXAMPLE 23.4

### Clinical and Chemical Analysis

Blood was collected from the monkeys in Experimental Example 23.1 one day before drug administration, and on days 1, 8, and 15 after drug administration. Here, the blood was collected in the same manner as in Experimental Example 23.3. The collected blood was subjected to clinical and chemical analysis using the Clinical Analyzer Model 7180 (Hitachi High-Technologies Corporation) for the items listed in Table 3 below.

TABLE 3

| Parameter | Abbr. | Unit | Method |
|---|---|---|---|
| Aspartate | AST | U/L | JSCC traceable method |
| aminotransferase | | | |
| Alanine | ALT | U/L | JSCC traceable method |
| aminotransferase | | | |
| Alkaline phosphatase | ALP | U/L | JSCC traceable method |
| Lactate dehydrogenase | LD | U/L | JSCC traceable method |
| Creatine kinase | CK | U/L | JSCC traceable method |
| Glucose | GLU | mg/dL | Enzymatic (Gluc-DH) |
| Total bilirubin | BIL | mg/dL | Enzymatic (BOD) |
| Urea nitrogen | UN | mg/dL | Enzymatic (urease-LEDH) |
| Creatinine | CRE | mg/dL | Enzymatic |
| Total cholesterol | CHO | mg/dL | Enzymatic (cholesterol |
| | | | oxidase) |
| Triglycerides | TG | mg/dL | Enzymatic (GK-GPO with |
| | | | free glycerol elimination) |
| Phospholipids | PL | mg/dL | Enzymatic (choline |
| | | | oxidase) |
| Inorganic phosphorus | IP | mg/dL | Enzymatic (maltose |
| | | | phosphorylase) |
| Calcium | CA | mg/dL | OCPC |
| Sodium | NA | mEq/L | Ion-selective electrode |
| Potassium | K | mEq/L | Ion-selective electrode |
| Chloride | CL | mEq/L | Ion-selective electrode |
| Total protein | TP | g/dL | Biuret |
| Albumin | ALB | g/dL | BCG |
| Albumin-globulin ratio | A/G | — | Calculated |

JSCC: Japan Society of Clinical Chemistry

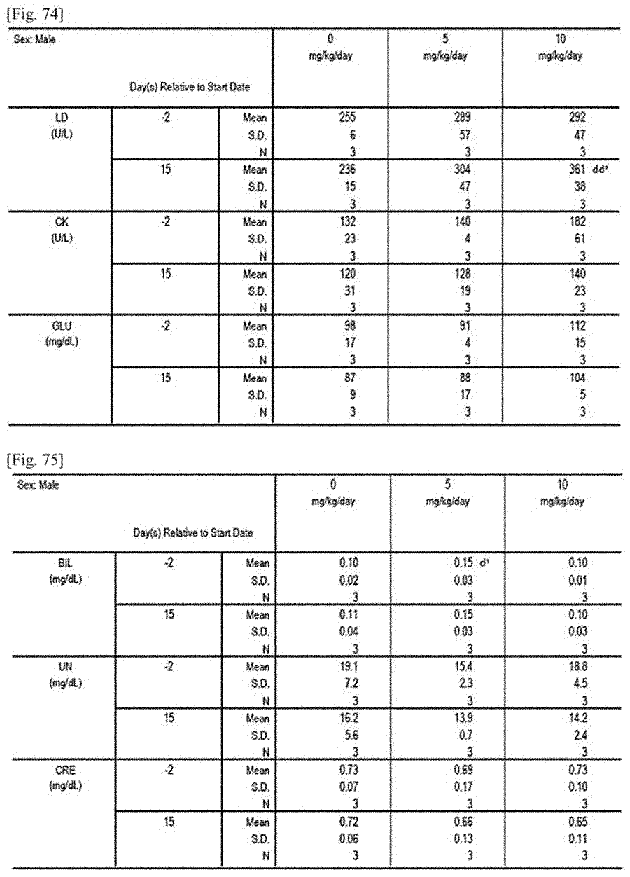

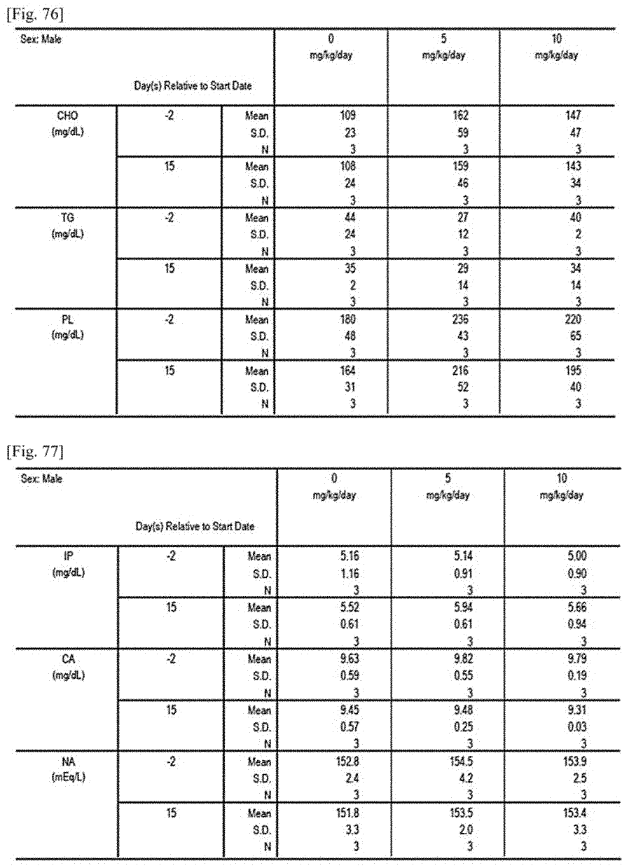

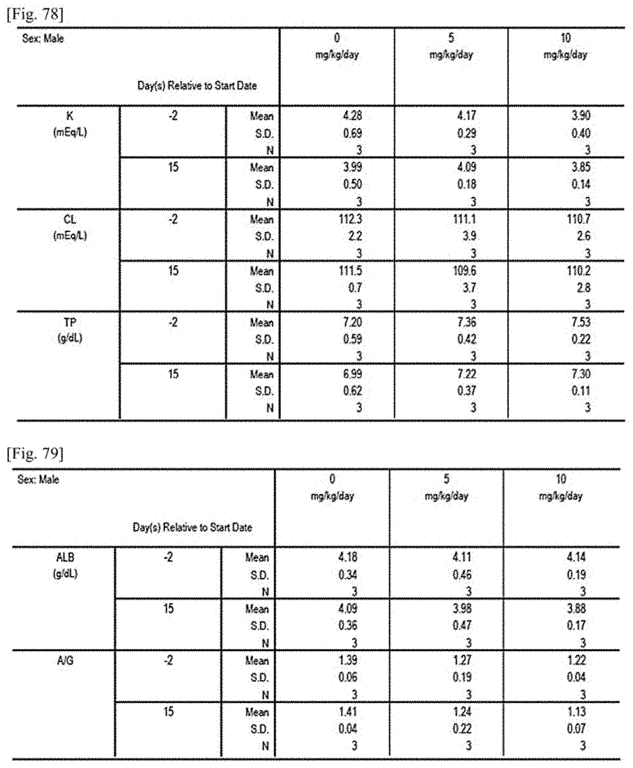

As a result, no toxicity caused by GI101 was detected in the clinical and chemical analysis (FIGS. 73 to 79).

### EXPERIMENTAL EXAMPLE 21.5

### Cytokine Analysis

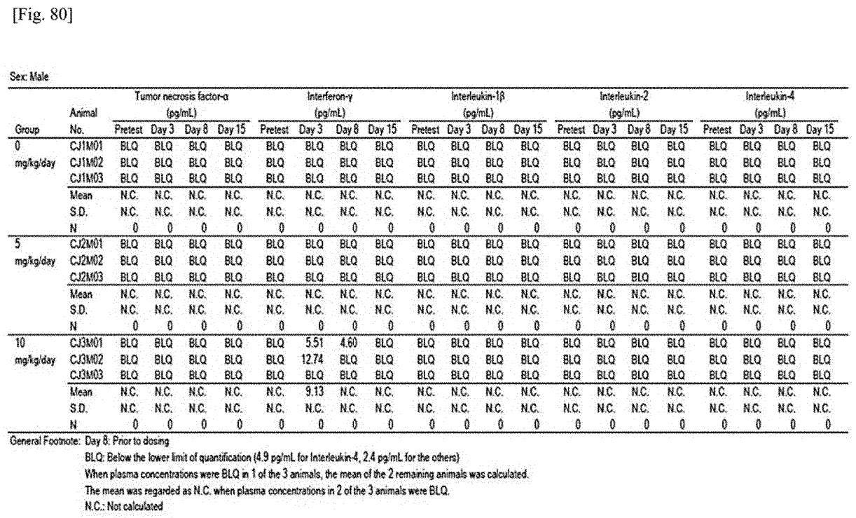

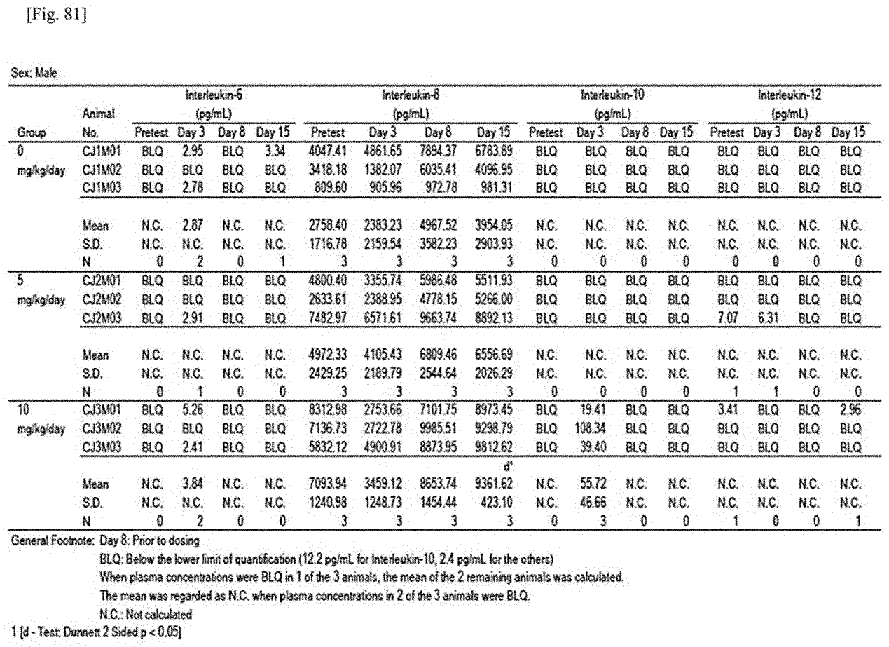

Blood was collected from the monkeys in Experimental Example 23.1 one day before drug administration, and on days 1, 8, and 15 after drug administration. Here, the blood was collected in the same manner as in Experimental Example 23.3. Using the Bio-Plex 200 (Bio-Rad Laboratories, Inc.) instrument and the Non-Human Primate Cytokine Magnetic Bead Panel (EMD Millipore) Assay Kit, the collected blood was analyzed for TNF-α, IFN-γ IL-1β, IL-2, IL-4, IL-6, IL-8, IL-10, and IL-12. As a result, no toxicity caused by GI101 was detected with respect to the cytokine analysis (FIGS. 80 and 81).

### EXPERIMENTAL EXAMPLE 23.6

### Immune Cell Analysis

Blood was collected from the monkeys in Experimental Example 23.1 one day before drug administration, and on days 1, 8, and 15 after drug administration. Here, the blood was collected in the same manner as in Experimental Example 23.3. Using a flow cytometer (LSRFortessa X-20, Becton, Dickinson and Company), the collected blood was analyzed for the following items:

1) Ki67+CD4: CD45+/CD3+/CD4+/Ki67+

2) Ki67+CD8: CD45+/CD3+/CD8+/Ki67+

3) Ki67+Treg: CD45+/CD3+/FoxP3+/Ki67+

4) Ki67+ICOS+Treg: CD45+/CD3+/FoxP3+/Ki67+/CD278+

5) ICOS+Treg: CD45+/CD3+/FoxP3+/CD278+

6) Ki67+NK cell: CD45+/CD16+ and CD56+/Ki67+.

As a result, in the immune cell analysis, all groups having received GI101 exhibited, on day 15, an increase in numbers of T cells, CD4+ T cells, CD8+ T cells, regulatory T cells, NK cells and Ki67+ T cells, Ki67+ CD4+ T cells, Ki67+ CD8+ T cells, Ki67+ regulatory T cells, Ki67+ ICOS+ regulatory T cells, Ki67+ NK cells, ICOS+ regulatory T cells.

Specifically, in lymphocytes, proportions of T cells, CD4+ T cells, regulatory T cells increased and a proportion of NK cells decreased, while a proportion of CD8+ T cells did not change. A proportion of regulatory T cells increased on day 3 and decreased on days 8 and 15. However, the proportion was still higher than the control group.

In addition, regarding proportions of immune cells, which are Ki67+, in the respective immune cells, proportions of Ki67+ T cells, Ki67+ CD4+ T cells, Ki67+ CD8+ T cells, Ki67+ regulatory T cells, Ki67+ ICOS+ regulatory T cells, Ki67+ NK cells, and ICOS+ regulatory T cells increased.

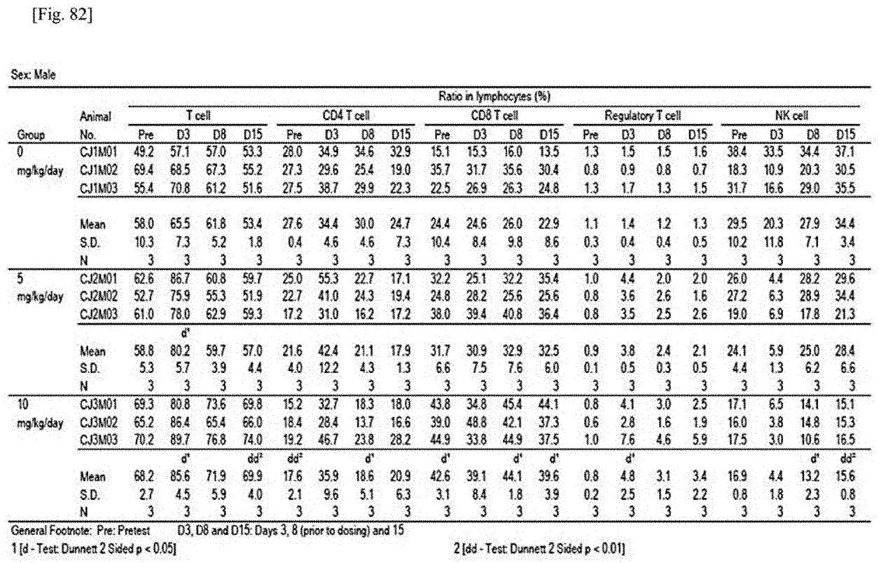

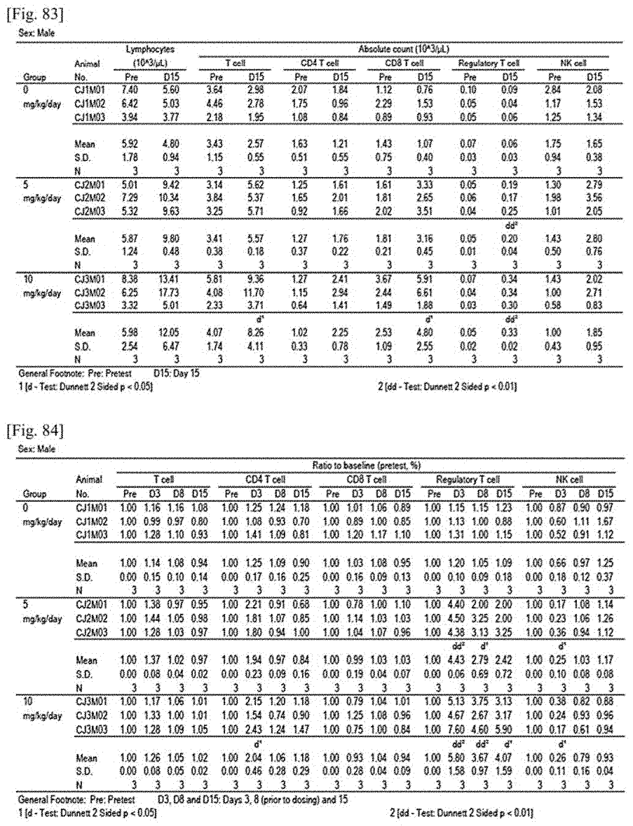

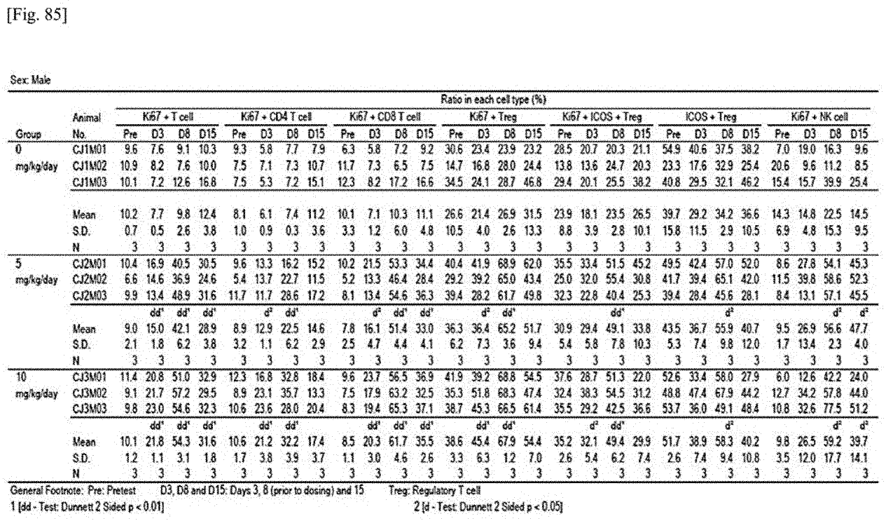

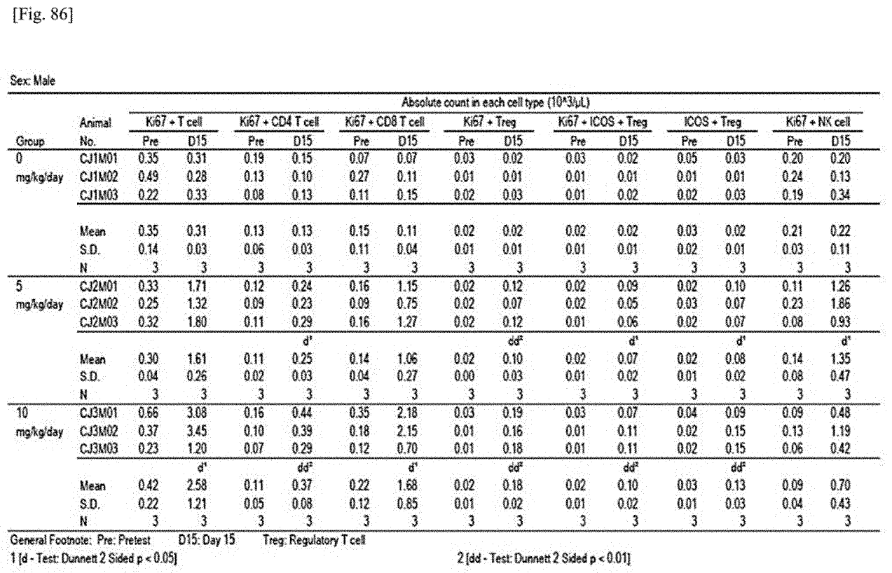

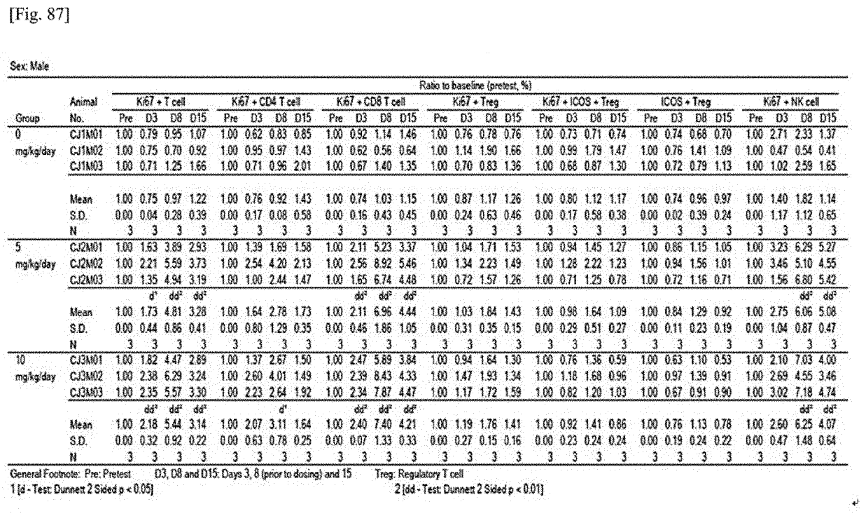

Furthermore, proportions of Ki67+ T cells, Ki67+ CD8+ T cells, and Ki67+ NK cells increased on days 3, 8, and 15; proportions of Ki67+ CD4+ T cells and Ki67+ regulatory T cells increased on days 3 and 8; and proportions of Ki67+ ICOS+ regulatory T cells and ICOS+ regulatory T cells increased only on day 8 (FIGS. 82 to 87).

### EXPERIMENTAL EXAMPLE 23.7

### Pathological Analysis

On day 16, the monkeys in Experimental Example 23.1 were sacrificed and all organs and tissues were fixed using 10% formalin. However, the testes were fixed using a formalin-sucrose-acetic acid (FSA) solution, and the eyes and optic nerve were fixed using 1% formaldehyde-2.5% glutaraldehyde in phosphate buffer. Hematoxylin-eosin staining was performed on the organs and tissues in the items listed in Table 4 below, and observations were made under an optical microscope.

TABLE 4

| | | | Specimen preparation | |
|---|---|---|---|---|
| | | Organ | HE- | |
| Organ/tissue | Fixation | weight | stained | Note |
| Heart | O | O | — | Left ventricular papillary |
| | | | | muscle, right ventricular |
| | | | | wall and areas including |
| | | | | the coronary artery and |
| | | | | aortic valve |
| Aorta (thoracic) | O | — | | |
| Sternum | O | — | | Decalcified |
| Sternal bone marrow | | — | | |
| Femurs | O | | | Distal articular cartilage |
| | (R&L) | | | and shaft; decalcified |
| Femoral bone marrow | O (R) | — | | Decalcified |
| Thymus | O | O | O | |
| Spleen | O | O | O | |
| Submandibular lymph | O | — | O | |
| nodes | | | | |
| Mesenteric lymph nodes | O | — | O | |
| Trachea | O | — | | Decalcified |
| Bronchi | O | O (R&L | — | Left anterior and right |
| Lungs | (R&L) | separated) | | posterior lobes |
| Tongue | O | — | | |
| Submandibular glands | O | O (R&L | | |
| | (R&L) | combined) | | |
| Parotid glands | O | — | | |
| | (R&L) | | | |
| Esophagus | O | — | | |
| Stomach | O | — | | Cardia, body and pylorus |
| Duodenum | O | — | | |
| Jejunum | O | — | | |
| Ileum | O | — | | |
| Peyer's patches | | | | |
| Cecum | O | — | | |
| Colon | O | — | | |
| Rectum | O | — | | |
| Liver | O | O | O | Left lateral lobe and right |
| Gallbladder | | (with bile- | O | medial lobe including the |
| | | drained | | gallbladder |
| | | gallbladder) | | |
| Pancreas | O | O | — | |
| Kidneys | O | O (R&L | O (R&L) | |
| | (R&L) | separated) | | |
| Urinary bladder | O | — | | |
| Pituitary | O | O | | |
| Thyroids | O | O (R&L | | |
| Parathyroids | (R&L) | separated) | | |
| Adrenals | O | O (R&L | | |
| | (R&L) | separated) | | |
| Testes | O | O (R&L | | |
| | (R&L) | separated) | | |
| Epididymides | O | O (R&L | | |
| | (R&L) | separated) | | |
| Prostate | O | O | | |
| Seminal vesicles | O | O | — | |
| Brain | O | O | — | Cerebrum (frontal, parietal |
| | | | | (including basal ganglia |
| | | | | and hippocampus) and |
| | | | | occipital lobes); |
| | | | | cerebellum; pons; and |
| | | | | medulla oblongata |
| Spinal cord (thoracic) | O | — | | |
| Sciatic nerve | O (L) | — | | |
| Eyes | O | — | | |
| | (R&L) | | | |
| Optic nerves | O | — | | |
| | (R&L) | | | |
| Lacrimal glands | O | — | | |
| | (R&L) | | | |
| Skeletal muscle (biceps | O (L) | — | | |
| femoris) | | | | |
| Skin (thoracic) | O | — | | |
| Injection site (tail vein) | O | — | | Decalcified |
| Skin of the thoracic or | O | — | — | |
| medial femoral region | | | | |
| with ID No. | | | | |

O: conducted
—: Not conducted
R&L: Both the right and left organs/tissues were conducted.
L: Either the right or left organ/tissue (usually the left) was conducted.
R: Either the right or left organ/tissue (usually the right) was conducted

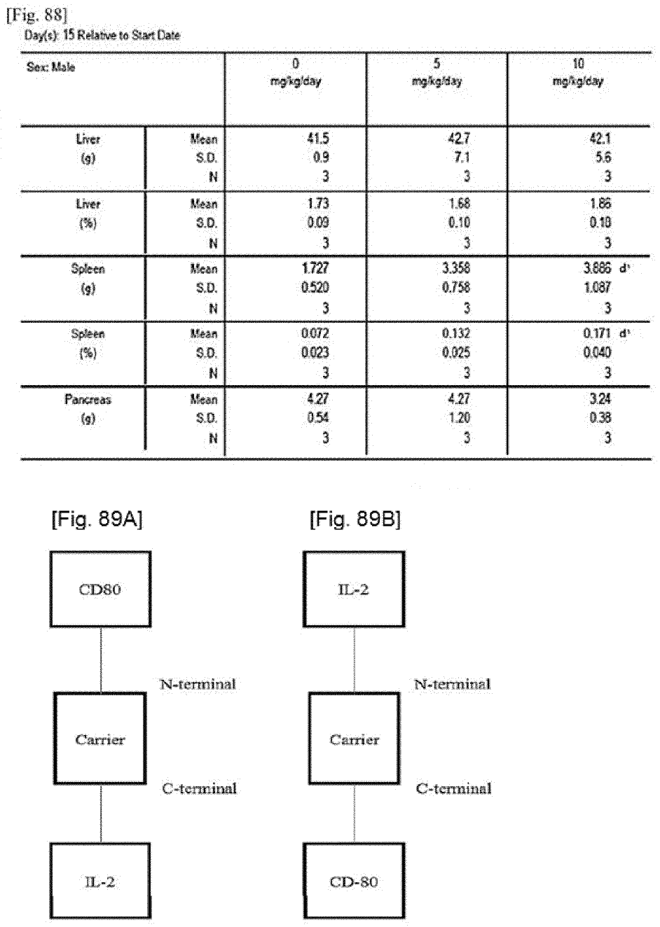

As a result, the group treated with GI101 at a dose of 5 mg/kg/day or 10 mg/kg/day exhibited an increase in spleen weight (FIG. 88). No significant changes were observed in the other tissues. In conclusion, in the groups having received GI101, some changes were observed but no toxicity was observed.

VI. Experimental Example 24 for Identifying Anticancer Effect of GI102. Identification of Anticancer Effect of GI102-M45

### EXPERIMENTAL EXAMPLE 24.1

### Identification of Anticancer Effect of GI102-M45 in Mice Transplanted with Mouse-Derived Colorectal Cancer Cells

5×10⁶ cells/0.05 ml of mouse-derived CT-26 cancer cell line were mixed with 0.05 ml Matrigel matrix phenol red-free (BD), and transplantation of the mixture was performed by subcutaneous administration at 0.1 ml in the right dorsal region of 6-week-old female BALB/c mice (Orient Bio). A certain period of time after the cancer cell transplantation, the tumor volume was measured and subjects that reached about 80 mm³ to 120 mm3 were separated. Then, the subjects were intravenously administered 0.1 ml of GI102-M45. A total of three administrations were given once every three days after the first administration, and PBS was given for a negative control. The tumor size was measured daily to identify an anticancer effect. Activity of GI102-M45 was identified in the same manner as in Experimental Example 16.

### EXPERIMENTAL EXAMPLE 24.2

### Identification of Anticancer Effect of GI102-M45 in Mice Transplanted with Mouse-Derived Lung Cells

C57BL/6 mice (female, 7-week-old) acquired from Orient Bio were subjected to an acclimation period of 7 days. Then, 5×10⁶ cells of LLC2 cancer cell line (ATCC, USA) were suspended in 0.1 ml PBS, and allotransplantation of the suspension was performed by subcutaneous administration at 0.1 ml in the right dorsal region of the mice. A certain period of time after the cancer cell transplantation, the tumor volume was measured and subjects that reached about 50 mm³ to 200 mm³ were selected, and then the selected mice were grouped evenly based on tumor size and body weight, each group containing 10 mice. Thereafter, using a disposable syringe (31G, 1 mL), no drug was administered to a negative control group, and an anti-PD-1 antibody at a dose of 5 mg/kg, or an anti-PD-1 antibody at a dose of 5 mg/kg and an anti-CTLA-4 antibody at a dose of 5 mg/kg were administered intravenously to positive control groups. For experimental groups, GI102-M45 at a dose of 0.1 mg/kg or 1 mg/kg was administered intravenously thereto. A total of three administrations were given once every three days after the first administration. The tumor size was measured daily. Activity of GI102-M45 was identified in the same manner as in Experimental Example 20.1.

### EXPERIMENTAL EXAMPLE 25

### Identification of Anticancer Effect of GI102-M61

### EXPERIMENTAL EXAMPLE 25.1

### Identification of Anticancer Effect of GI102-M61 in Mice Transplanted with Mouse-Derived Colorectal Cancer Cells

5×10⁶ cells/0.05 ml of mouse-derived CT-26 cancer cell line were mixed with 0.05 ml Matrigel matrix phenol red-free (BD), and transplantation of the mixture was performed by subcutaneous administration at 0.1 ml in the right dorsal region of 6-week-old female BALB/c mice (Orient Bio). A certain period of time after the cancer cell transplantation, the tumor volume was measured and subjects that reached about 80 mm³ to 120 mm³ were separated. Then, the subjects were intravenously administered 0.1 ml of GI102-M61. A total of three administrations were given once every three days after the first administration, and PBS was given to a negative control. The tumor size was measured daily to identify an anticancer effect. Activity of GI102-M61 was identified in the same manner as in Experimental Example 16.

### EXPERIMENTAL EXAMPLE 25.2

### Identification of Antitumor Effect of GI102-M61 in Mice Transplanted with Mouse-Derived Lung Cancer Cells

C57BL/6 mice (female, 7-week-old) acquired from Orient Bio were subjected to an acclimation period of 7 days. Then, 5×10⁶ cells of LLC2 cancer cell line (ATCC, USA) were suspended in 0.1 ml PBS, and allotransplantation of the suspension was performed by subcutaneous administration at 0.1 ml in the right dorsal region of the mice. A certain period of time after the cancer cell transplantation, the tumor volume was measured and subjects that reached about 50 mm³ to 200 mm³ were selected, and then the selected mice were grouped evenly based on tumor size and body weight, each group containing 10 mice. Thereafter, using a disposable syringe (31G, 1 mL), no drug was administered to a negative control group, and an anti-PD-1 antibody at a dose of 5 mg/kg, or an anti-PD-1 antibody at a dose of 5 mg/kg and an anti-CTLA-4 antibody at a dose of 5 mg/kg were administered intravenously to positive control groups. For experimental groups, GI102-M61 at a dose of 0.1 mg/kg or 1 mg/kg was administered intravenously thereto. A total of three administrations were given once every three days after the first administration. The tumor size was measured daily. Activity of GI102-M61 was identified in the same manner as in Experimental Example 20.1.

### EXPERIMENTAL EXAMPLE 26

### Identification of Anticancer Effect of GI102-M72

### EXPERIMENTAL EXAMPLE 26.1

### Identification of Antitumor Effect of GI102-M72 in Mice Transplanted with Mouse-Derived Colorectal Cancer Cells

5×10⁶ cells/0.05 ml of mouse-derived CT-26 cancer cell line were mixed with 0.05 ml Matrigel matrix phenol red-free (BD), and transplantation of the mixture was performed by subcutaneous administration at 0.1 ml in the right dorsal region of 6-week-old female BALB/c mice (Orient Bio). A certain period of time after the cancer cell transplantation, the tumor volume was measured and subjects that reached about 80 mm³ to 120 mm³ were separated. Then, the subjects were intravenously administered 0.1 ml of GI102-M72. A total of three administrations were given once every three days after the first administration, and PBS was given to a negative control. The tumor size was measured daily to identify an anticancer effect. Activity of GI102-M72 was identified in the same manner as in Experimental Example 16.

### EXPERIMENTAL EXAMPLE 26.2

### Identification of Anticancer Effect of GI102-M72 in Mice Transplanted with Mouse-Lung Cancer Cells

C57BL/6 mice (female, 7-week-old) acquired from Orient Bio were subjected to an acclimation period of 7 days. Then, 5×10⁶ cells of LLC2 cancer cell line (ATCC, USA) were suspended in 0.1 ml PBS, and allotransplantation of the suspension was performed by subcutaneous administration at 0.1 ml in the right dorsal region of the mice. A certain period of time after the cancer cell transplantation, the tumor volume was measured and subjects that reached about 50 mm³ to 200 mm³ were selected, and then the selected mice were grouped evenly based on tumor size and body weight, each group containing 10 mice. Thereafter, using a disposable syringe (31G, 1 mL), no drug was administered to a negative control group, and an anti-PD-1 antibody at a dose of 5 mg/kg, or an anti-PD-1 antibody at a dose of 5 mg/kg and an anti-CTLA-4 antibody at a dose of 5 mg/kg were administered intravenously to positive control groups. For experimental groups, GI102-M72 at a dose of 0.1 mg/kg or 1 mg/kg was administered intravenously thereto. A total of three administrations were given once every three days after the first administration. The tumor size was measured daily. Activity of GI102-M72 was identified in the same manner as in Experimental Example 20.1.

VII. Identification of GI101 and GI 102 with High Content of Sialic Acid and Anticancer Effect Thereof

### PREPARATION EXAMPLE 11

### Preparation of GI101 and GI102 with High Content of Sialic Acid

### PREPARATION EXAMPLE 11.1

### Preparation of GI101 with High Content of Sialic Acid

As described in Preparation Example 1, a fusion protein was produced using a stable cell line expressing the polypeptide of SEQ ID NO: 9.

In a fed-batch culture manner, (i) the cell line was fed every other day at a feed medium supply ratio of 7.0% (v/v) from day 3 to day 11 and the culture solution was recovered on day 12, (ii) the cell line was fed every other day at a feed medium supply ratio of 7.0% (v/v) and the culture solution was recovered on day 8, or (iii) the cell line was fed every other day at a feed medium supply ratio of 7.0% (v/v) from day 3 to day 7 and the culture solution was recovered on day 7.

The fusion protein was purified from the recovered culture solution by chromatography. In order to measure the content of sialic acid contained in the glycan structure of the fusion protein, the purified fusion protein was treated with a sialic acid degrading enzyme, sialidase, to isolate sialic acid. The isolated sialic acid was detected and quantified using HPLC (Waters Corp.). It was confirmed that the content of sialic acid in the fusion protein was about 7.7 mol/mol for (i), about 15.37 mol/mol for (ii), and about 19.8 mol/mol for (iii), respectively, and they were designated as GI-101_SA (7.7), GI-101_SA (15.37), and GI-101_SA (19.80), respectively.

### PREPARATION EXAMPLE 11.2

### Preparation of GI102 with High Content of Sialic Acid

As described in Preparation Example 8, a fusion protein was produced using a stable cell line expressing the polypeptide of SEQ ID NO: 28.

In a fed-batch culture manner, (iv) the cell line was fed every other day at a feed medium supply ratio of 7.0%(v/v) and the culture solution was recovered on day 9, or (v) the cell line was fed every other day at a feed medium supply ratio of 7.0%(v/v) from day 3 to day 11 and the culture solution was recovered on day 12.

The fusion protein was purified from the recovered culture solution by chromatography. In order to measure the content of sialic acid contained in the glycan structure of the fusion protein, the purified fusion protein was treated with a sialic acid degrading enzyme, sialidase, to isolate sialic acid. The isolated sialic acid was detected and quantified using HPLC (Waters Corp.). It was confirmed that the content of sialic acid in the fusion protein was about 19.0 mol/mol for (iv) and about 25 mol/mol for (v), respectively, and they were designated as GI-102_SA (19) and GI-102_SA (25), respectively.

### EXPERIMENTAL EXAMPLE 27

### Identification of Efficacy of GI-101 Depending on Content of Sialic Acid

### EXPERIMENTAL EXAMPLE 27.1

### Experimental Group and Dosage

33 to 55-month-old male Philippine monkeys (Cynomolgus monkeys) were prepared, and the experiment was carried out in Genia Inc. (Gyeonggi-do, Korea). The experimental procedure described in the experimental protocol was reviewed and approved by the Institutional Animal Care and Use Committee (IACUC) of Orient Bio before conducting the experiment. Proleukin (Novartis, hIL-2) was used as a control.

Experimental groups and drug dosages are summarized in Table 5.

TABLE 5

| | Route of | | Dose | |
|---|---|---|---|---|
| | admini- | | (mg/ | Administration |
| Group | stration | Substance | kg) | cycle |
| G1 | I.V. | Vehicle | 0 | once every 3 weeks, |
| | | (Formulation | | a total of 2 cycles |
| | | buffer) | | |
| G2 | | GI-101_SA | 1 | once every 3 weeks, |
| | | (7.7) | | a total of 2 cycles |
| G3 | | GI-101_SA | 1 | once every 3 weeks, |
| | | (15.37) | | a total of 2 cycles |
| G4 | | GI-101_SA | 2.5 | once every 3 weeks, |
| | | (15.37) | | a total of 2 cycles |
| G5 | | GI-101_SA | 1 | once every 3 weeks, |
| | | (19.80) | | a total of 2 cycles |
| G6 | | GI-101_SA | 2.5 | once every 3 weeks, |
| | | (19.80) | | a total of 2 cycles |
| G7 | S.C. | Proleukin | 0.1 | administered daily |
| | | | | on days 1 to 5 every |
| | | | | 3 weeks, a total of 2 |
| | | | | cycles |

### EXPERIMENTAL EXAMPLE 27.2

### Hematological Changes Following Administration of GI-101

### EXPERIMENTAL EXAMPLE 27.2.1

### Blood Collection

About 1 ml of blood was collected from the femoral vein of the monkey using a disposable syringe (3 ml, 23G, Korea Vaccine, KOR). The blood was put into an SST tube (BD VACUTAINER®, REF367955, BD Medical, USA), mixed thoroughly, and incubated at room temperature for 30 minutes to 1 hour. Thereafter, blood was centrifuged at 3,000 rpm at 4° C. for 15 minutes (COMBI R515, HANIL, KOR) to isolate serum. The isolated serum was dispensed into tubes and temporarily stored in a deep freezer (IBK-U500, InfoBiotech, KOR) at −70±10° C. Blood collection was carried out at the following times for all experimental subjects:

Days 1 (pre-dose, Pre), 3, 6, 9, 11, 15, 22 (pre-dose, Pre), 24, 27, 30, 32, and 36 from blood collection day.

### EXPERIMENTAL EXAMPLE 27.2.2

### Isolation of PBMC

About 3 ml of blood was collected from the femoral vein of the monkey (Cynomolgus monkey) using a disposable syringe (5 ml, 23G, Korea Vaccine, KOR). The collected blood was put in K2 EDTA (BD VACUTAINER®, BD Medical, USA) and incubated at room temperature, and then 3 ml of Dulbecco's phosphate-buffered saline (DPBS, Gibco, USA) was mixed. The mixture was slowly loaded into a tube containing 6 ml of Ficoll (HISTOPAQUE®-1077, Sigma, USA) to form a layer, and then centrifugation (Combi R757, Hanil) was carried out at 400×g for 30 minutes at 20° C. without artificial deceleration. After centrifugation, the resulting buffy coat was transferred to a new tube. At this time, serum components were minimized. The buffy coat transferred to a new tube was washed with DPBS and then washed with RPMI 1640 (Hyclone, USA). After washing, the total number of peripheral blood mononuclear cells (PBMCs) was counted and diluted with freezing media (CRYOSTOR® CS10, Stemcell Technologies, CAN) to 1×10⁶ cells/lml/vial. The diluted PBMCs were dispensed into tubes and temporarily stored in a deep freezer (IBK-U500, InfoBiotech, KOR) at −70±10° C. for 1-2 days, then transferred to a liquid nitrogen container and stored.

### EXPERIMENTAL EXAMPLE 27.2.3

### Effects of GI-101 by Content of Sialic Acid on Proliferation of Lymphocytes, CD8+ T Cells and NK Cells

As described in Experimental Example 27.2.2, isolated PBMCs were prepared, and the total number of cells was measured using a hemocytometer.

Specifically, the cell pellet from which the supernatant was removed was washed with a FACS buffer (1% FBS in PBS), and then reacted with an Fc blocker (BD, Cat No. 564220) at 4° C. for 30 minutes. Thereafter, it was treated with an antibody cocktail containing BV510 anti-CD3 Ab (BD, Cat No. 740187), PE-CF594 anti-CD8 Ab (BD, Cat No. 562282), PerCP anti-CD14 Ab (Biolegend, Cat No. 301848), PerCP anti-CD20 Ab (BD, Cat No. 566132) and APC anti-NKG2A Ab (Beckman Coulter, Cat No. A60797), reacted at 4° C. for 30 minutes, and then washed with a FACS buffer. Thereafter, it was treated with the fixable viability dye eFluor 780 (Invitrogen, Cat No. 65-0865-14), reacted at 4° C. for 30 minutes, and then washed with a FACS buffer. The cell pellet from which the supernatant was removed was reacted with 200 μL of a fixation/permeabilization working solution (Invitrogen, Cat No. 00-5523-00) for 30 minutes in a light-blocked environment, and then washed with a permeabilization buffer (Invitrogen, Cat No. 00-5523-00). If intracellular staining is required, it was treated with 100 μL of an antibody diluted in a permeabilization buffer and reacted for 30 minutes at room temperature in a light-blocked environment. After the reaction was completed, it was washed twice with a permeabilization buffer, resuspended in a permeabilization buffer, and the number of lymphocytes, CD8+ T cells and NK cells was analyzed using CYTEK AURORA (CYTEK, Fremont, Calif.) and FlowJo software. The information of the antibody used for each cell analysis is as follows.

Lymphocytes: BV510 anti-CD3 Ab (BD, Cat No. 740187; SP34-2)

CD8+ T cells: BV510 anti-CD3 Ab (BD, Cat No. 740187; SP34-2), PE-CF594 anti-CD8 Ab (BD, Cat No. 562282; RPA-T8), PerCP anti-CD14 Ab (Biolegend, Cat No. 301848; M5E2), PerCP anti-CD20 Ab (BD, Cat No. 566132; 2H7)

NK cells: BV510 anti-CD3 Ab (BD, Cat No. 740187L SP34-2), CD14 Ab (Biolegend, Cat No. 301848; M5E2), PerCP anti-CD20 Ab (BD, Cat No. 566132; 2H7) and APC anti-NKG2A Ab (Beckman Coulter, Cat No. A60797; Z199)

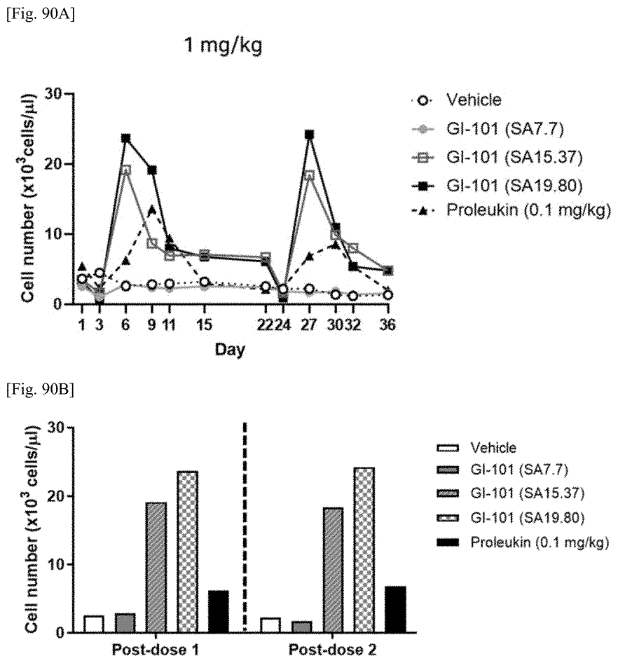

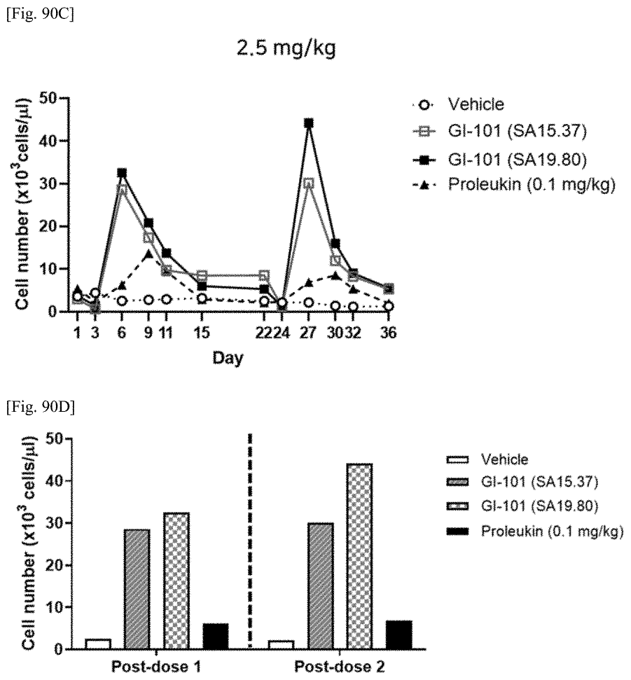

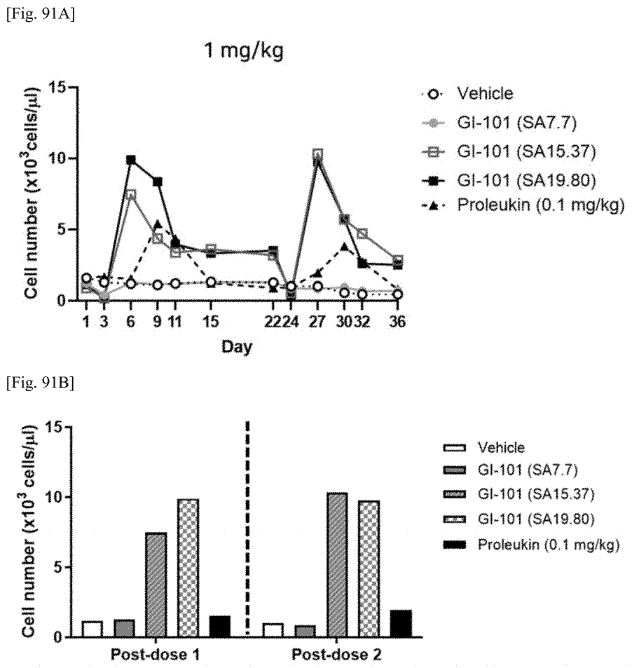

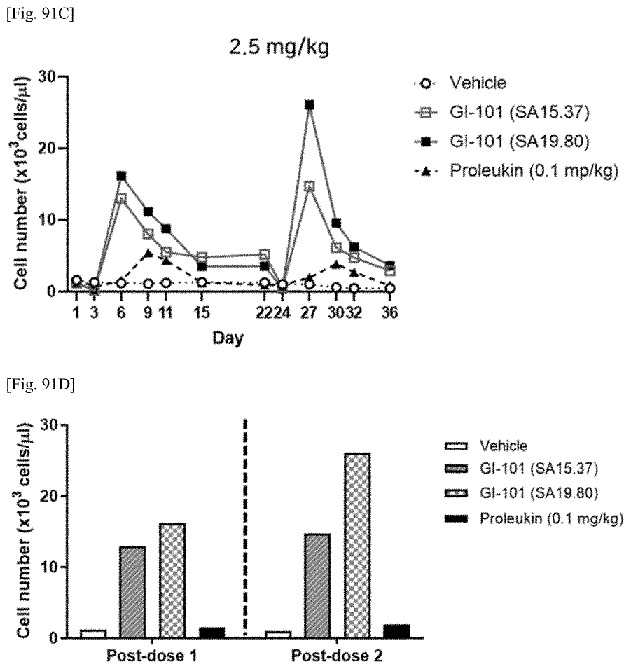

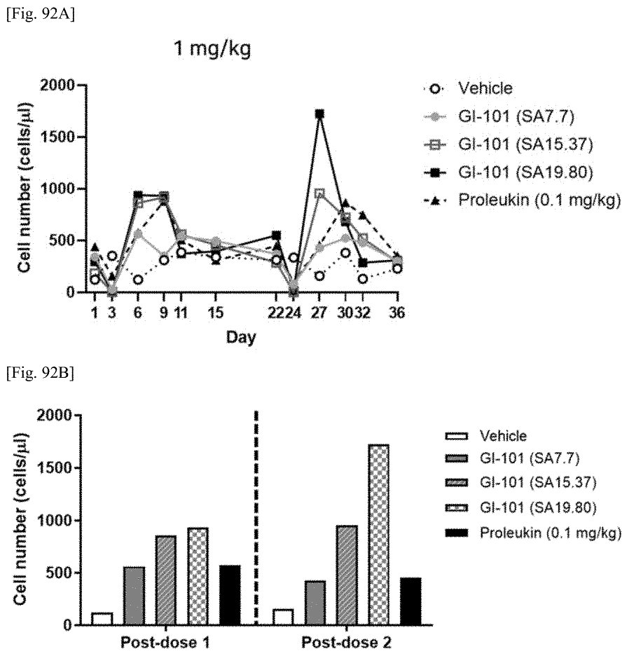

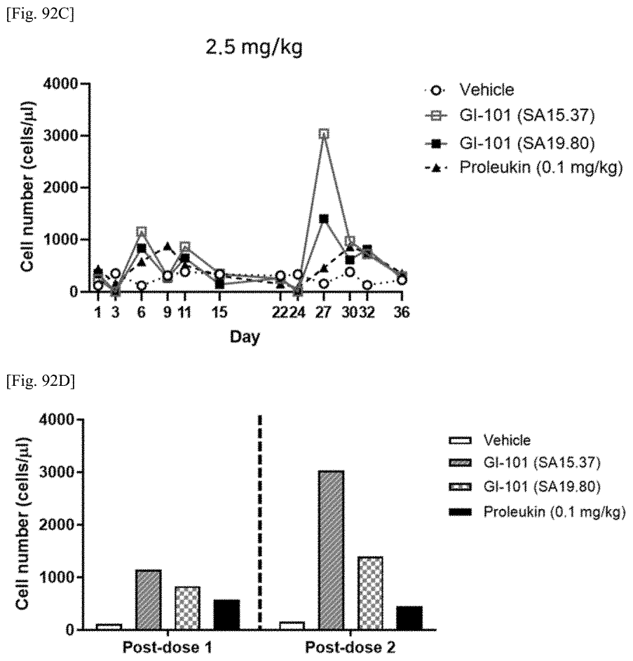

The results obtained by measuring the number of lymphocytes when administration of GI-101 at a dose of 1 mg/kg are shown in FIGS. 90A and 90B, and the results obtained by measuring the number of lymphocytes when administration of GI-101 at a dose of 2.5 mg/kg are shown in FIGS. 90C and 90D. The results obtained by measuring the number of CD8+ T cells when administration of GI-101 at a dose of 1 mg/kg are shown in FIGS. 91A and 91B, and the results obtained by measuring the number of CD8+ T cells when administration of GI-101 at a dose of 2.5 mg/kg are shown in FIGS. 91C and 91D. The results obtained by measuring the number of NK cells when administration of GI-101 at a dose of 1 mg/kg are shown in FIGS. 92A and 92B, and the results obtained by measuring the number of NK cells when administration of GI-101 at a dose of 2.5 mg/kg are shown in FIGS. 92C and 92D.

As a result, when GI-101 at a dose of 1 mg/kg was administered, the proliferation of lymphocytes, CD8+ T cells and NK cells was induced in a sialic acid content-dependent manner (FIGS. 90A, 90B, 91A, 91B, 92A and 92B). In addition, when GI-101 at a dose of 2.5 mg/kg was administered, the proliferation of lymphocytes and CD8+ T cells was induced in a sialic acid content-dependent manner, and the induction of NK cells was not shown to be increased in a sialic acid content-dependent manner, but higher immune cell proliferation ability than under Proleukin administration conditions was identified (FIGS. 90C, 90D, 91C, 91D, 92C and 92D).

### EXPERIMENTAL EXAMPLE 27.3

### Hematological Changes Following Aministration of GI-102_SA (19)

### EXPERIMENTAL EXAMPLE 27.3.1

### Experimental Groups and Dosages

As described in Experimental Example 27.1., male Philippine monkeys (Cynomolgus monkeys) were prepared and the experiment was carried out. Proleukin (Novartis, hIL-2) was used as a control.

Experimental groups and drug dosages are summarized in Table 6.

TABLE 6

| | Route of | | Dose | |
|---|---|---|---|---|
| | admini- | | (mg/ | |
| Group | stration | Substance | kg) | Administration cycle |
| G1 | I.V. | Vehicle | 0 | once every 3 weeks, |
| | | (Formulation | | a total of 2 cycles |
| | | buffer) | | |
| G2 | | GI-102_SA | 1 | once every 3 weeks, |
| | | (19) | | a total of 2 cycles |
| G3 | | GI-102_SA | 2.5 | once every 3 weeks, |
| | | (19) | | a total of 2 cycles |
| G4 | S.C. | Proleukin | 0.1 | administered daily |
| | | | | on days 1 to 5 every |
| | | | | 3 weeks, a total of 2 |
| | | | | cycles |

### EXPERIMENTAL EXAMPLE 27.3.2

### Changes in Lymphocyte, CD8+ T Cell and NK Cell Proliferation Following Administration of GI-102_SA (19)

The number of lymphocytes, CD8+ T cells and NK cells following administration of GI-102_SA (19) was measured in the same manner as in Experimental Example 27.2.3.

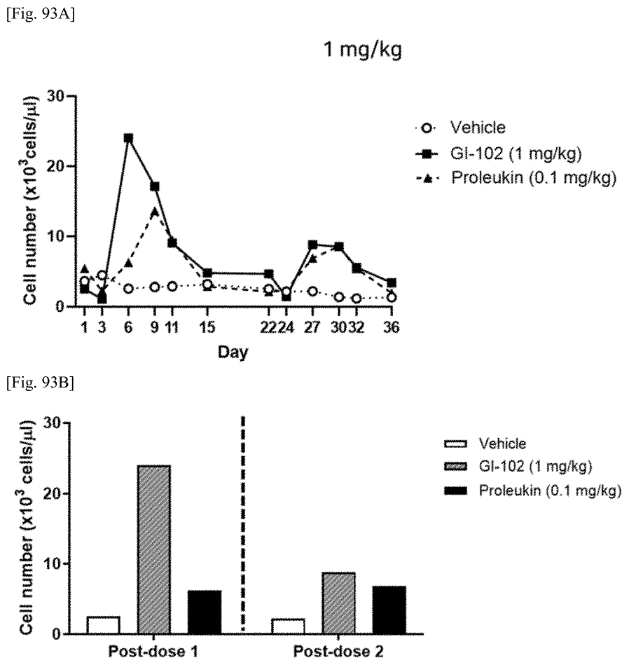

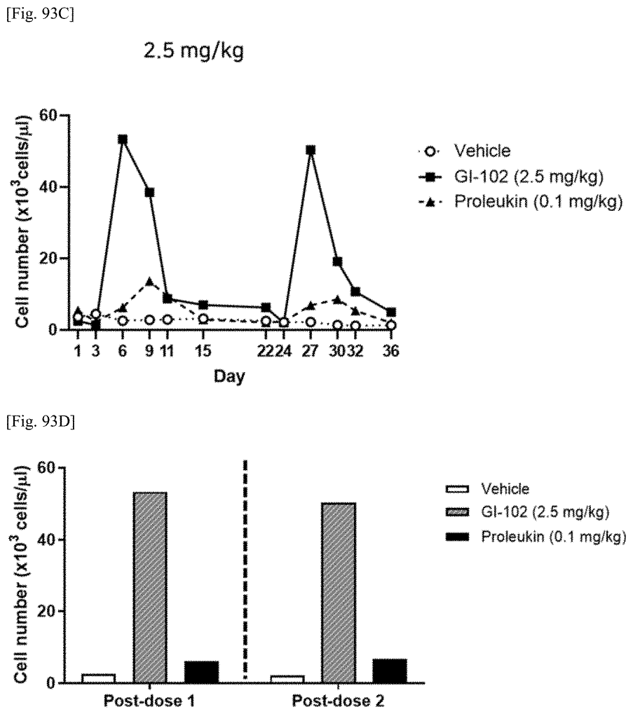

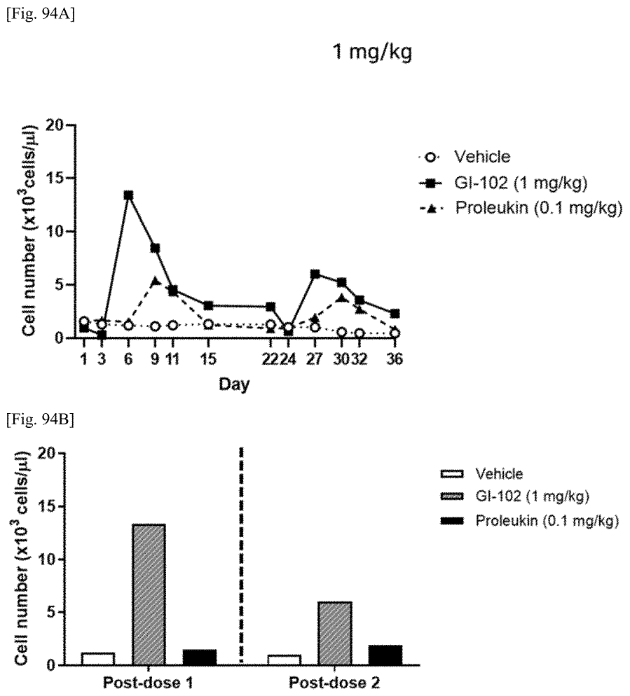

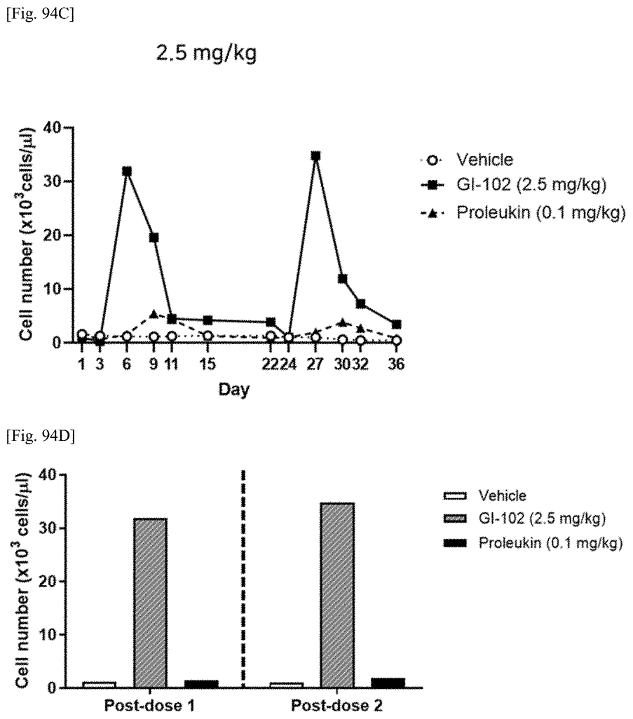

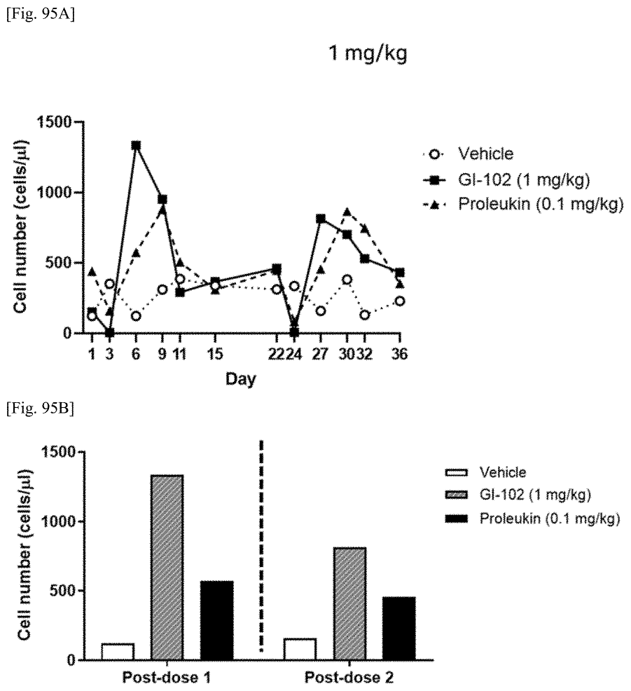

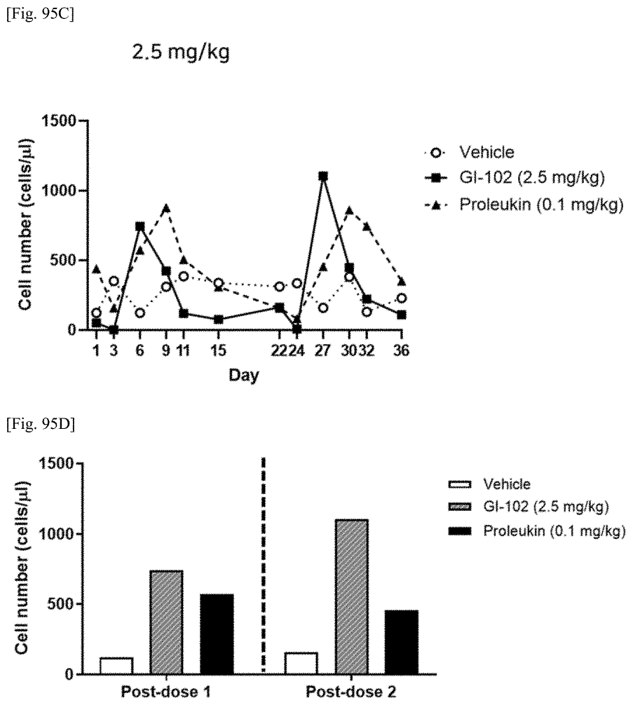

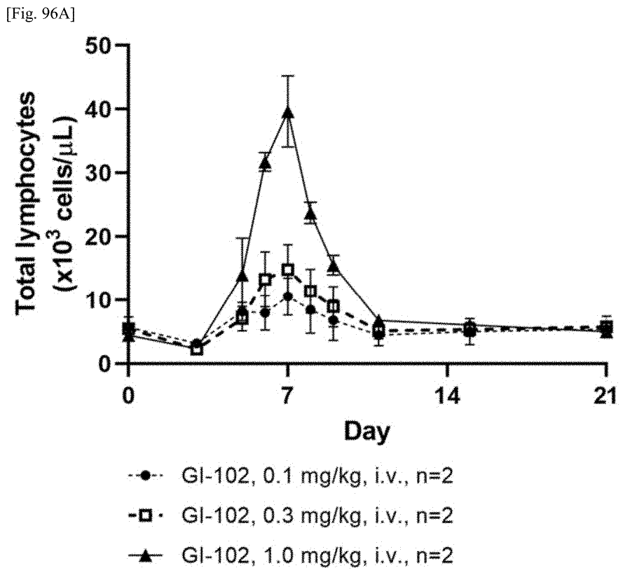

The results obtained by measuring the number of lymphocytes when administration of GI-102_SA (19) at a dose of 1 mg/kg are shown in FIGS. 93A and 93B, and the results obtained by measuring the number of lymphocytes when administration of GI-102_SA (19) at a dose of 2.5 mg/kg are shown in FIGS. 93C and 93D. The results obtained by measuring the number of CD8+ T cells when administration of GI-102_SA (19) at a dose of 1 mg/kg are shown in FIGS. 94A and 94B, and the results obtained by measuring the number of CD8+ T cells when administration of GI-102_SA (19) at a dose of 2.5 mg/kg are shown in FIGS. 94C and 94D. The results obtained by measuring the number of NK cells when administration of GI-102_SA (19) at a dose of 1 mg/kg are shown in FIGS. 95A and 95B, and the results obtained by measuring the number of NK cells when administration of GI-102_SA (19) at a dose of 2.5 mg/kg are shown in FIGS. 95C and 95D.

As a result, it was identified that GI-102_SA (19) activated the proliferation of lymphocytes, CD8+ T cells and NK cells in vivo compared to Proleukin. In particular, GI-102_SA (19) showed the effect of maximally increasing the number of lymphocytes, CD8+ T cells and NK cells on day 6 based on the time point of administration, and at this time point, a higher level of immune cell proliferation effect than that of Proleukin was identified (FIGS. 93A to 95D).

### EXPERIMENTAL EXAMPLE 27.4

### Hematological Changes Following Administration of GI-102_SA (25)

### EXPERIMENTAL EXAMPLE 27.4.1

### Experimental Group and Dosage

As described in Experimental Example 27.1., male Philippine monkeys (Cynomolgus monkeys) were prepared and the experiment was carried out.

Experimental groups and drug dosages are summarized in Table 7.

TABLE 7

| | Route of | | Dose | |
|---|---|---|---|---|
| | admini- | | (mg/ | |
| Group | stration | Substance | kg) | Administration cycle |
| G1 | I.V. | GI-102_SA | 0.1 | once every 3 weeks, a |
| | | (25) | | total of 1 cycle |
| G2 | | GI-102_SA | 0.3 | once every 3 weeks, a |
| | | (25) | | total of 1 cycle |
| G3 | | GI-102_SA | 1 | once every 3 weeks, a |
| | | (25) | | total of 1 cycle |

### EXPERIMENTAL EXAMPLE 27.4.2

### Changes in Lymphocyte, CD8+ T Cell and NK Cell Proliferation Following Administration of GI-102_SA (25)

The number of lymphocytes, CD8+ T cells and NK cells following administration of GI-102_SA (25) was measured in the same manner as in Experimental Example 27.2.3.

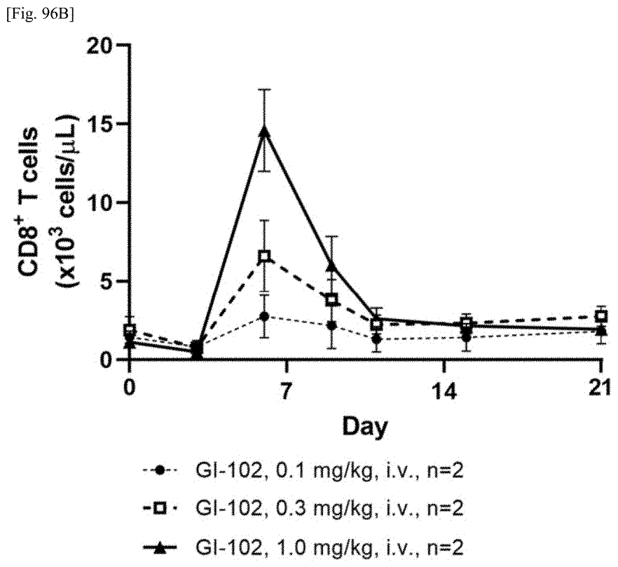

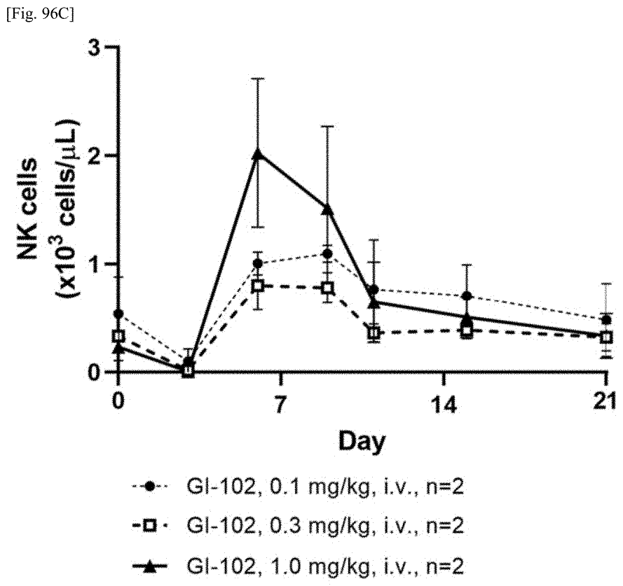

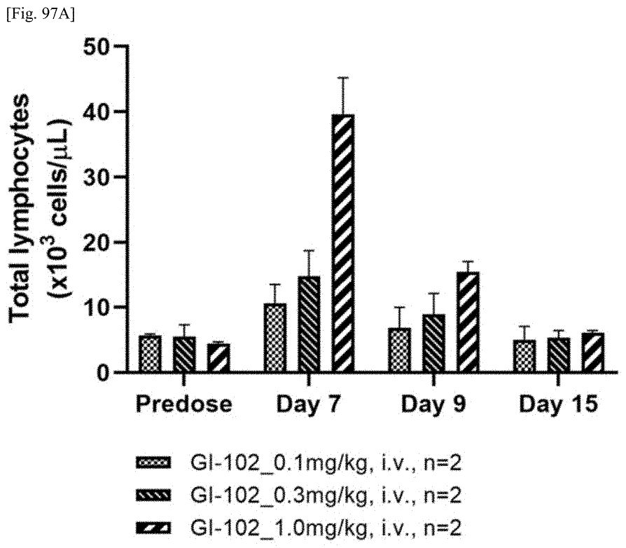

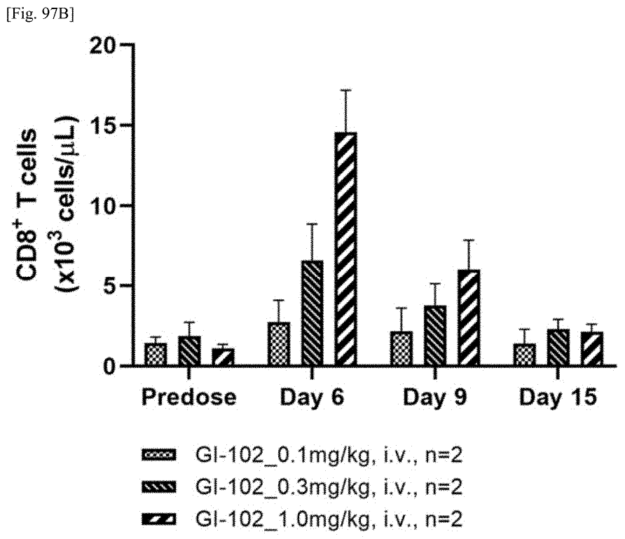

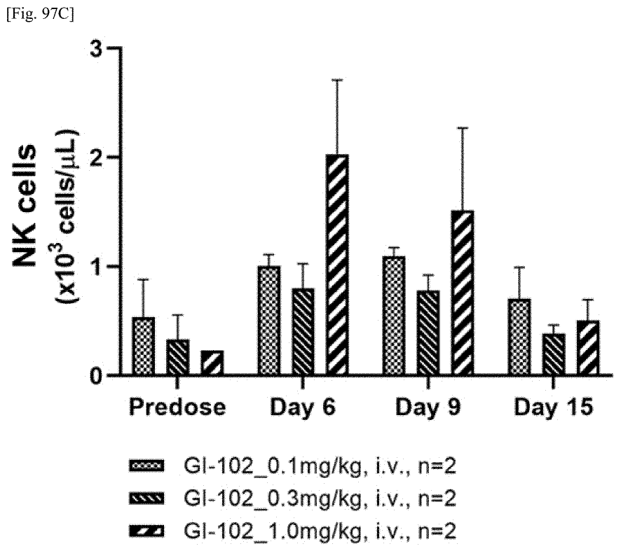

The results obtained by measuring the number of lymphocytes, CD8+ T cells and NK cells depending on the administered dose of GI-102_SA (25) are shown in FIGS. 96A, 96B and 96C, respectively. In addition, the results obtained by measuring the number of lymphocytes, CD8+ T cells and NK cells on days 0 (pre-dose), 7, 9 and 15 depending on the administered dose of GI-102_SA (25) are shown in FIGS. 97A, 97B, and 97C, respectively.

As a result, when GI-102_SA (25) was administered, the proliferation of lymphocytes, CD8+ T cells and NK cells was induced in a dose-dependent manner. In particular, the number of lymphocytes was shown to be the maximum on day 7 based on the time point of administration, and the number of CD8+ T cells and NK cells was shown to be the maximum on day 6 (FIGS. 96A to 97C).

1. A fusion protein dimer, comprising:
two monomers, each of which contains the following structural formula (I) or (II):
N′-X-[linker (1)]n-Fc domain-[linker (2)]m-Y-C′ (I)
N′-Y-[linker (1)]n-Fc domain-[linker (2)]m-X-C′ (II)
in the structural formulas (I) and (II),
N′ is the N-terminus of the fusion protein,
C′ is the C-terminus of the fusion protein,
X is a CD80 protein,
Y is an IL-2 protein,
the linkers (1) and (2) are peptide linkers, and
n and m are each independently 0 or 1,
wherein the fusion protein dimer comprising sialic acid and a molar ratio of the sialic acid to the fusion protein dimer is at least 7.

2. The fusion protein dimer of claim 1, wherein n and m are each independently 1.

3. The fusion protein dimer of claim 1, wherein the IL-2 protein is an IL-2 variant.

4. The fusion protein dimer of claim 3, wherein the IL-2 variant is obtained by substitution of at least one selected from the 38th, 42nd, 45th, 61st, and 72nd amino acids in the amino acid sequence of SEQ ID NO: 10.

5. The fusion protein dimer of claim 3, wherein the IL-2 variant comprises at least one substitution selected from the group consisting of R38A, F42A, Y45A, E61R, and L72G in the amino acid sequence of SEQ ID NO: 10.

6. The fusion protein dimer of claim 3, wherein the IL-2 variant comprises any one selected from the following substitution combinations (a) to (d) in the amino acid sequence of SEQ ID NO: 10:
(a) R38A/F42A
(b) R38A/F42A/Y45A
(c) R38A/F42A/E61R
(d) R38A/F42A/L72G.

7. The fusion protein dimer of claim 3, wherein the IL-2 variant comprises the amino acid sequence of SEQ ID NO: 6, 22, 23, or 24.

8. The fusion protein dimer of claim 1, wherein the CD80 protein is a CD80 fragment.

9. The fusion protein dimer of claim 8, wherein the CD80 fragment consists of the 35th amino acid to 242^{nd} amino acid in the amino acid sequence of SEQ ID NO: 11.

10. The fusion protein dimer of claim 1, wherein the Fc domain is a wild type or variant.

11. The fusion protein dimer of claim 10, wherein the variant of the Fc domain comprises the amino acid sequence of SEQ ID NO: 12.

12. The fusion protein dimer of claim 1, wherein the linker (1) is a peptide linker consisting of the amino acid sequence of SEQ ID NO: 3; and wherein the linker (2) is a peptide linker consisting of the amino acid sequence of SEQ ID NO: 5.

13. The fusion protein dimer of claim 1, wherein the fusion protein comprises a sequence identity of 90% or higher to the amino acid sequence of SEQ ID NO: 9, 26, 28, or 30.

14. The fusion protein dimer of claim 1, wherein the sialic acid is N-acetylneuraminic acid.

15. The fusion protein dimer of claim 1, wherein the molar ratio of sialic acid to the fusion protein dimer is from 7 to 25, or from 15 to 30.

16. The fusion protein dimer of claim 1, wherein the fusion protein dimer is a homodimer.

17. A pharmaceutical composition comprising the fusion protein dimer of claim 1.

18. A method for enhancing immunity in a subject, comprising:
administering the fusion protein dimer according to claim 1 or a pharmaceutical composition comprising the fusion protein to the subject in need thereof.

19. The method of claim 18, wherein the fusion protein proliferates any lymphocyte.

20. The method of claim 19, wherein the lymphocyte is any one selected from the group consisting of a CD8+ cytotoxic T cell, a CD4+ T helper cell, a regulatory T cell, a natural killer cell, and a B cell.
